(19) United States
(12) Patent Application Publication
(10) Pub. No.: US 2026/0152763 A1
(43) Pub. Date: Jun. 4, 2026

(54) CENTRIFUGAL CELL POSITIONING SYSTEM AND METHODS

(71) Applicant: SPOT BIOSYSTEMS LTD., Palo Alto, CA (US)

(72) Inventors: Ly James LEE, Columbus, OH (US); Junfeng SHI, Columbus, OH (US); Shih-Chung YU, Palo Alto, CA (US)

(21) Appl. No.: 18/707,122

(22) PCT Filed: Nov. 4, 2022
(86) PCT No.: PCT/US2022/049054

§ 371 Date: May 2, 2024

Related U.S. Application Data
(60) Provisional application No. 63/276,422, filed on Nov. 5, 2021.

(51) Int. Cl. C12N 15/87; B04B 5/04; C12M 1/26; C12M 1/42; C12N 13/00

(52) U.S. Cl. CPC C12N 15/87; B04B 5/0414; C12M 33/10; C12M 35/02; C12N 13/00

(57) ABSTRACT

Provided are systems and methods for positioning cells in proximity to pores of an electroporation chip. Cell positioning in proximity to pores of an electroporation chip may be achieved using centrifugal forces to improve transfection efficiency.

### CROSS REFERENCE

This application claims the benefit of U.S. Provisional Application Ser. No. 63/276,422 filed Nov. 5, 2021 which is hereby incorporated by reference in its entirety.

### BACKGROUND

During electroporation, an electrical field is applied to cells to increase the permeability of the cell membrane. Electroporation may be used to allow chemicals, drugs, DNA, plasmid DNAs, RNAs, proteins, other charged biomolecules, charged molecules, and particulates to be introduced into the cell by electrotransfer or electrotransfection.

### SUMMARY

This disclosure generally provides devices, methods, and systems for electroporating cells. Provided herein are methods of electroporating cells comprising: (a) providing a suspension comprising a plurality of cells to a first surface of an electroporation chip; (b) applying a centrifugal force to the plurality of cells, such that at least a portion of the plurality of cells is pressed against the first surface of the electroporation chip; and (c) providing an electrical voltage across the electroporation chip as the centrifugal force is applied to the plurality of cells. In some cases, the electric voltage is provided when the first surface of the electroporation chip is about perpendicular to an axis of the centrifugal force. In some cases, applying a centrifugal force to the plurality of cells flattens the portion of the plurality of cells against the electroporation chip. In some cases, applying a centrifugal force to the plurality of cells elongates the portion of the plurality of cells against the electroporation chip. In some cases, the centrifugal force causes the cells to elongate or flatten, thereby improving contact of the cells with apertures of the pores in the chip.

In some cases, providing the suspension comprises injecting the suspension into a cell chamber adjacent to the first surface of the electroporation chip. In some cases, the suspension is injected into the cell chamber using a syringe.

In some cases, the centrifugal force is applied using a centrifuge. In some cases, the centrifuge comprises a rotor, and applying the centrifugal force further comprises rotating the rotor at approximately 100 to 2000 RCF.

In some cases, the plurality of cells comprises a plurality of eukaryotic cells. In some cases, the plurality of eukaryotic cells comprises mammalian cells. In some cases, the mammalian cells are selected from the group consisting of human cells, rodent cells, and non-human primate cells. In some cases, the plurality of eukaryotic cells comprises a plurality of cells from a cell line. In some cases, the plurality of cells from a cell line is a plurality of cells from a suspension cell line or an adherent cell line. In some cases, the suspension cell line is selected from the group consisting of a cell line of a myeloma origin, a cell line of a lymphoma origin, a cell line of and a leukemia origin. In some cases, the suspension cell line is a human cell line. In some cases, the suspension cell line is an animal cell line. In some cases, the plurality of eukaryotic cells is selected from the group consisting of mouse embryonic fibroblasts (MEF), human embryonic fibroblasts (HEF), dendritic cells, mesenchymal stem cells, bone marrow-derived dendritic cells, bone marrow derived stromal cells, adipose stromal cells, enucleated cells, neural stem cells, immature dendritic cells, and immune cells. In some cases, the plurality of eukaryotic cells is a suspension cell line selected from the group consisting of NS0, U937, HL60, WEHI231, YAC1, U266B1, Jurkat, and THP-1.

In some cases, the first surface of the electroporation chip comprises a plurality of pores. In some cases, each pore (or at least one) of the plurality of pores comprises a diameter of about 50 nanometers to about 10 micrometers. In some cases, the current is applied at a voltage of about 1V to about 1500V. In some cases, individual pores within the plurality of pores comprises a diameter within a range of about 50 nanometers to about 10 micrometers.

In some cases, the method further comprises (d) removing the suspension after providing the electrical voltage across the electroporation chip as the centrifugal force is applied to the plurality of cells. In some cases, the method comprises repeating (a) to (d). In some cases, the method comprises varying a voltage of the electrical current between repeating (a) to (d). In some cases, the method comprises varying a duration of the electrical current between repeating (a) to (d).

In some cases, the providing the electrical voltage occurs after centrifugal movement resulting from the centrifugal force stops. In some cases, the providing the electrical voltage occurs after centrifugal movement resulting from the centrifugal force begins to stop. In some cases, the providing the electrical voltage occurs after centrifugal movement resulting from the centrifugal force begins to stop. In some cases, the providing the electrical voltage occurs after the centrifugal movement resulting from the centrifugal force slows down to a velocity less than 50%, less than 25 %, or less than 10% of a highest velocity the centrifugal movement once reaches. In some cases, the providing the electrical voltage occurs after the plurality of cells are elongated. In some cases, the providing the electrical voltage occurs after the plurality of cells are elongated in the absence of the centrifugal force. In some cases, the providing the electrical voltage occurs after the plurality of cells are flattened. In some cases, the providing the electrical voltage occurs after the plurality of cells are flattened in the absence of the centrifugal force. In some cases, the providing the electrical voltage occurs simultaneously with the centrifugal force being applied. In some cases, the providing the electrical voltage occurs after the initiation of application of the centrifugal force. In some cases, the providing the electrical voltage occurs after the initiation of application of the centrifugal force and before the centrifugal force is stopped. In some cases, the providing the electrical voltage occurs after the initiation of application of the centrifugal force and when the centrifugal force is stopped or after the centrifugal force is stopped. In some cases, the providing the electrical voltage occurs when the application of the centrifugal force is initiated. In some cases, the providing the electrical voltage occurs when the application of the centrifugal force is initiated and before the centrifugal force is stopped. In some cases, the providing the electrical voltage occurs when the application of the centrifugal force is initiated and when the centrifugal force is stopped or after the centrifugal force is stopped.

Provided herein are cases of a centrifuge for providing an electrical current to a suspension within one or more centrifuge tubes, the centrifuge comprising: a rotor comprising a hub; at least one tube holder connected to the hub; and a circuit for providing an electric current and voltage through at least one of the one or more centrifuge tubes or through all of the one or more centrifuge tubes.

In some cases, each centrifuge tube (or at least one) of the one or more centrifuge tubes comprises a first electrode and a second electrode for electric communication with the circuit. In some cases, the at least one tube holder is rotatable about the hub. In some cases, the at least one tube holder pivots under influence of a centrifugal force applied by the rotor. In some cases, the electric current and voltage is provided through each centrifuge tube of the one or more centrifuge tubes when a center axis of a centrifuge tube is about aligned with an axis of the centrifugal force applied to the one or more centrifuge tubes.

In some cases, each centrifuge tube (or at least one) of the one or more centrifuge tubes comprises a first electrical contact, and wherein the circuit comprises a second electrical contact, wherein the first electrical contact and the second electrical contact are in electrical communication when the center axis of the centrifuge tube is about aligned with the axis of the centrifugal force applied on the one or more centrifuge tubes. In some cases, the circuit is connected to a power source. In some cases, the power source is external to the centrifuge. In some cases, centrifuge further comprises a connector to provide electrical communication between the power source and the circuit. In some cases, the power source is provided within the centrifuge. In some cases, the power source supplies a voltage of about 1V to about 1500V to the circuit. In some cases, the rotor rotates at approximately 100 to 2000 RCF.

Provided herein are cases of an electroporation system comprising: one or more centrifuge tubes, each centrifuge tube or at least one centrifuge tube comprising: a first tube, an electroporation chip disposed within the first tube, a first electrode provided on a first side of the electroporation chip, and a second electrode provided on a second side of the electroporation chip; a centrifuge device comprising: a rotor comprising a hub; one or more tube holders coupled to the hub; and a circuit for providing an electric current to the one or more centrifuge tubes provided within the one or more tube holders; and a power source for supplying the electric current to the circuit.

In some cases, at least one of, or each of the one or more tube holders are pivotably coupled to the hub such that the one or more tube holders rotate towards a about horizontal orientation as the rotor is rotated. In some cases, one or more first contacts of the centrifuge contact the first electrode of each of (or at least one of) the one or more centrifuge tubes when the rotor is rotated. In some cases, the rotor is rotated at approximately 100 to 2000 RCF.

In some cases, the system further comprises a cell chamber incorporated on the first side of the electroporation chip. In some cases, the first electrode is provided within the cell chamber. In some cases, the system further comprises a first electrode wire for operatively connecting the first electrode to a first electrode contact, wherein the first electrode contact is provided on an exterior of the first tube.

In some cases, at least one centrifuge tube, or each centrifuge tube, of the one or more centrifuge tubes comprises a removable cap. In some cases, an aperture is provided through the removable cap, such that the first electrode wire passes through the aperture of the removable cap. In some cases, at least one centrifuge tube, or each centrifuge tube further comprises a stabilizer, wherein the stabilizer comprises an electrode wire aperture such that the first electrode wire passes through the electrode wire aperture to provide an electrical connection from the first electrode to the first electrode contact. In some cases, at least one centrifuge tube, or each centrifuge tube of the one or more centrifuge tubes further comprises the stabilizer that holds the first electrode wire in place. In some cases, at least one centrifuge tube, or each centrifuge tube, of the one or more centrifuge tubes further comprises a second tube, wherein the second tube comprises a stabilizer which abuts the cell chamber and provides a physical barrier between the second tube and the cell chamber. In some cases, the first electrode is provided within the cell chamber.

In some cases, the centrifuge comprises a plurality of tube holders for simultaneous electroporation of one or more samples provided in a plurality of centrifuge tubes.

In some cases, provided herein is a method of using the electroporation system, wherein the method comprises providing a suspension comprising a plurality of cells to the first side of the electroporation chip, applying a centrifugal force to the plurality of cells, such that at least a portion of the plurality of cells is pressed against the first surface of the electroporation chip; and providing an electrical voltage across the electroporation chip as the centrifugal force is applied to the plurality of cells.

Provided herein are centrifuge tubes comprising: a first tube; an electroporation chip disposed within the first tube; a first electrode provided on a first side of the electroporation chip; and a second electrode provided on a second side of the electroporation chip, wherein the first electrode and the second electrode provide an electric field across the electroporation chip.

In some cases, the centrifuge tube further comprises a cell chamber incorporated on the first side of the electroporation chip. In some cases, the first electrode is provided within the cell chamber. In some cases, the centrifuge tube further comprises a first electrode wire for operatively connecting the first electrode to a first electrode contact, wherein the first electrode contact is provided on an exterior of the first tube. In some cases, the centrifuge tube further comprises a removable cap. In some cases, an aperture is provided through the removable cap, such that the first electrode wire passes through the aperture of the removable cap. In some cases, the centrifuge tube further comprises a stabilizer, wherein the stabilizer comprises an electrode wire aperture such that the first electrode wire passes through the electrode wire aperture to provide an electrical connection from the first electrode to the first electrode contact. In some cases, the stabilizer holds the first electrode wire in place.

In some cases, the centrifuge tube further comprises a second tube, wherein the second tube comprises a stabilizer which abuts the cell chamber and provides a physical barrier between the second tube and the cell chamber.

In some cases, the first electrode is provided within the cell chamber. In some cases, the centrifuge tube further comprises a first electrode wire for operatively connecting the first electrode to a first electrode contact, wherein the first electrode contact is provided on an exterior of the first tube. In some cases, the centrifuge tube further comprises a second electrode contact electrically coupled to the second electrode and provided on an exterior to the first tube. In some cases, the second electrode contact is provided on a side of the first tube opposite side of first electrode contact.

In some instances, the first electrode is a positive electrode and wherein the second electrode is a negative electrode. In some cases, the first electrode is a cathode, and the second electrode is an anode.

In some cases, the removable cap comprises a syringe aperture for receiving a syringe to provide a plurality of cells to the cell chamber. In some cases, the syringe aperture is positioned off a center axis of the removable cap. In some cases, the stabilizer comprises a syringe through hole for receiving the syringe to provide cells to the cell chamber. In some cases, a friction fit is provided between the stabilizer and the first tube. In some cases, the friction fit facilitates retention of the second tube within the first tube.

In some cases, the stabilizer provides a liquid tight seal of the cell chamber. In some cases, the stabilizer provides an air-tight seal of the seal chamber.

In some cases, a distal portion of the first tube comprises a transfection reagent chamber to receive a transfection reagent. In some cases, the transfection reagent comprises a plasmid, DNA, RNA, protein, other charged biomolecules, charged molecules, charged particulates, or a combination thereof.

In some cases, the electroporation chip provides a physical barrier between the transfection chamber and the cell chamber. In some cases, the second electrode is provided within the transfection reagent chamber.

In some cases, the electroporation chip is removable. In some cases, the first tube comprises an open end and a closed end.

In some cases, a transfection reagent chamber is located between the second side of the electroporation chip and the closed end of the first tube. In some cases, the second electrode is within the transfection reagent chamber. In some cases, the centrifuge tube comprises a removable cap. In some cases, the open end of the first tube is threaded to receive the removable cap. In some cases, the centrifuge tube comprises a first electrode wire for operatively connecting the first electrode to a first electrode contact, wherein the first electrode contact is provided on an exterior of removeable cap, and wherein an aperture is provided through the removable cap, such that the first electrode wire passes through the aperture of the removable cap. In some cases, the centrifuge tube further comprises a second electrode contact electrically coupled to the second electrode and provided at the exterior of the first tube and proximal to the closed end of the first tube.

Provided herein are cases of a method of electroporating cells comprising: providing a suspension comprising a plurality of cells adjacent to an electroporation chip; elongating the plurality of cells, such that at least a portion of the plurality of cells is pressed against a first surface of the electroporation chip; and providing an electrical current across the electroporation chip.

Provided herein are cases, of method of electroporating cells comprising: providing a suspension comprising a plurality of cells adjacent to an electroporation chip; flattening the plurality of cells, such that at least a portion of the plurality of cells is pressed against a first surface of the electroporation chip; and providing an electrical current across the electroporation chip.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative cases, in which the principles of the invention are utilized, and the accompanying drawings of which:

FIGS. 1A-1G depict diagrams of different parts of a prototype centrifuge for nano-or micro-electroporation (N/MEP) or “centrifuge-N/MEP.” FIG. 1A shows a diagram of a modified centrifuge tube for the centrifuge-N/MEP. FIG. 1B shows a diagram of a circuit installed in the modified centrifuge tubes attached to a rotation frame. FIG. 1C shows a connecting diagram between an external power supply and a modified centrifugation machine, with zoomed-in diagrams for the slip ring connector (SRC) and the configuration of the electrodes and the chamber containing plasmid solution in the N/MEP centrifuge tube.

FIG. 1D shows a diagram of the cell positioning under the influence of centrifugal force.

FIG. 1E shows distribution of the cells to be electroporated and the plasmids. FIG. 1F shows a diagram of a modified centrifugation machine for the centrifuge-N/MEP with the lid opened. FIG. 1G shows a diagram of a modified centrifugation machine for the centrifuge-N/MEP with the lid closed.

FIGS. 2A-2E show photos of the prototype centrifuge-N/MEP. FIG. 2A shows the full centrifuge-N/MEP device. FIG. 2B shows the inner side of the device. FIG. 2C shows the cover of the device. FIG. 2D shows an assembled centrifuge tube for the centrifuge-N/MEP. FIG. 2E shows a disassembled centrifuge tube for the centrifuge-N/MEP with the second tube and electroporation chip removed from the first tube.

FIGS. 3A-3F depict the effect of centrifugation on N/MEP. FIG. 3A shows the distribution of electrical field varied with different locations relative to the nanopores. FIG. 3B shows a quantification of electrical fields with different locations relative to the nanopores with cells with different gap from the nanopores. FIG. 3C shows a diagram of the effect of centrifugation on the gap between a cell and a nearby nanopore. FIG. 3D shows overlayed images of bright field and florescence after the electroporation of FAM-oligodeoxynucleotides (ODNs) under various centrifugation and electroporation conditions. FIG. 3E shows a diagram of the morphology changes of two types of cells in response to centrifugation. FIG. 3F shows a quantification of contact area changes in response to centrifugation among the two types of cells.

FIGS. 4A-4F depict the electroporation of FAM-ODNs or GFP with the centrifuge-N/MEP. FIG. 4A shows the electroporation of FAM-ODNs with the centrifuge-N/MEP under different electroporation conditions during or after centrifuge. FIG. 4B shows the quantification of the percentage of the transfected cells and the fluorescence intensity of the transfected cells from FIG. 4A. FIG. 4C shows the electroporation of FAM-ODNs with the centrifuge-N/MEP during or after centrifuge. FIG. 4D shows the electroporation of GFP with the centrifuge-N/MEP under different electroporation conditions. FIG. 4E shows the quantification of the fluorescence intensity of the transfected cells from FIG. 4D. FIG. 4F shows the electroporation of GFP with the centrifuge-N/MEP under different electroporation voltages.

FIG. 5A-5B depict the effect of the chip materials on transfection efficiency. FIG. 5A shows SEM images of track-etched membrane for localized electroporation. FIG. 5B shows a comparison of transfection efficiency between the N/MEP chips using track-etched membrane Transwell (TEP) versus silicon wafer-based N/MEP chips.

FIG. 6A-6B depict the application of centrifuge-N/MEP in the production of p53 containing extracellular vesicles (EVs). FIG. 6A shows the production of EVs under different electroporation conditions of centrifuge-N/MEP. FIG. 6B shows distribution of mRNA content in the transfected cells versus in the released EVs under different electroporation conditions of centrifuge-N/MEP.

### DETAILED DESCRIPTION

Provided herein are centrifuge tubes, centrifuges, electroporation centrifuge systems and methods of electroporating cells that offer high yield and high-throughput delivery of biomolecules into cells. In some cases, the systems and methods enable intracellular delivery of nucleic acids, or other biomolecules, into high quantities of cells, such as over 200,000 cells per square centimeter (cm²), often in relatively short time periods, such as a few minutes. The tubes, centrifuges, systems and methods described herein may allow for rapid cell loading, uniform nano-electroporation, and fast post-transfection cell collection. In some cases, cell positioning against nano-or micro-sized pores on a nano-or micro-electroporation (N/MEP) chip is achieved by centrifugal forces. The systems, compositions, devices, and methods herein may be applied to both adherent and suspension cells. As such, the systems, compositions, devices, and methods herein may be applied regardless of cellular anchor properties. Experimental results provided herein show that applying centrifugal forces to cells to be electroporated can significantly improve N/MEP-based transfection efficiency. In many cases, the methods, devices and systems provided herein can achieve uniform and precise delivery of various cargos from small oligodeoxynucleotides to large plasmid DNAs and nanoparticles.

### I. Centrifuge Tubes

This disclosure provides centrifuge tubes, including centrifuge tubes with unique features. Generally, the centrifuge tubes comprise or contain additional elements such as an electroporation chip in order to effectuate electroporation of cells contemporaneously with centrifugation. In some cases, a centrifuge tube provided herein comprises one or more additional tubes (e.g., inner tubes). In some cases, the centrifuge tube (sometimes referred to herein as an “outer tube”) or the one or more additional tubes (e.g., inner tube[s]) comprise additional elements such as electrodes, electrode wiring or conducting elements, syringe features, stabilizing features (e.g., stabilizer), and/or sample input tube. In some cases, a stabilizing feature spatially positions an input tube, an electrode wire and/or other feature.

In some instances, described herein is a centrifuge tube comprising: a first tube, an electroporation chip disposed within the first tube, a first electrode, and a second electrode, wherein the first electrode and the second electrode provide an electric field across the electroporation chip. In some cases, the first electrode is provided on a first side of the electroporation chip, and/or the second electrode is provided on a second side of the electroporation. In some cases, the centrifuge tube described herein further comprises a second tube, a cell chamber, a transfection reagent chamber, a syringe, a stabilizer, and/or a cap, or any combination thereof. In some cases, the centrifuge tube is an outer tube that holds the second tube. In such cases, the centrifuge tube may comprise an electroporation chip (e.g., an electroporation chip with microchannels or nanochannels); a user may add transfection reagent to the bottom of the tube, such that the reagent contacts one face of the electroporation chip, while the other face of the electroporation chip may be a cell chamber that contacts cells. The second tube may comprise a stabilizer element, an electrode, and/or a syringe. The syringe may be used to introduce cells into the cell chamber of the electroporation chip. The stabilizer may be used to stabilize or position the syringe and/or the electrode.

In some cases, the centrifuge tube described herein comprises a first tube 110 configured for electroporation of a plurality of cells. The centrifuge tube can hold a certain volume of liquid. In some cases, the centrifuge tube is a 50 milliliter (50 mL) tube. In some cases, the centrifuge tube is a 25 ml tube, a 15 ml tube, a 10 ml tube, a 1 ml tube, or tube of any other volume. In some cases, the volume of the centrifuge tube is more than 1 ml, 10 ml, 15 ml, 25 ml, or 50 ml. In some cases, the volume of the centrifuge tube is less than 1 ml, 10 ml, 15 ml, 25 ml, or 50 ml.

In some cases, the first tube 110, comprises a threaded circumference at a one end to receive a cap 115. In some cases, the centrifuge tube is conical at one end (such as the bottom of the tube), opposite of the threaded circumference. In some cases, the centrifuge tube has a flat bottom; in some cases, the centrifuge tube has a pointed bottom. In some cases, the centrifuge tube has a rounded bottom.

The centrifuge tubes provided herein, including the outer tubes (e.g., first tubes) and/or the inner tubes can be made of any non-conductive material, e.g., glass, polymer (e.g., polyethylene, polypropylene), plastic, ceramic, metal. In some specific cases, the centrifuge tube comprises polypropylene. In some specific cases, the centrifuge tube comprises fluoroelastomer. In some specific cases, the centrifuge tube comprises neoprene. In some specific cases, the centrifuge tube comprises nitrile. In some specific cases, the centrifuge tube comprises nylon. In some specific cases, the centrifuge tube comprises polyethylene. In some specific cases, the centrifuge tube comprises polytetrafluoroethylene. In some specific cases, the first tube comprises polyurethane. In some specific cases, the centrifuge tube comprises polyvinyl chloride. In other cases, the centrifuge tube comprises glass. In other cases, the centrifuge tube comprises ceramic. In other cases, the centrifuge tube comprises metal.

The cylindrical region of the outer tube can have any diameter. In some cases, the cylindrical region of the outer tube has a diameter of about 10, 20, 30, 40, 50 or up to 100 mm. The cylindrical region of the inner tube can have any diameter. In some cases, the cylindrical region of the inner tube has a diameter of about 10, 20, 30, 40, 50 or up to 100 mm. In some cases, the cylindrical region of the outer tube has a diameter of more than 10, 20, 30, 40, 50, or 100 mm. In some cases, the cylindrical region of the outer tube has a diameter of less than 10, 20, 30, 40, 50, or 100 mm. In some cases, the cylindrical region of the inner tube has a diameter of more than 10, 20, 30, 40, 50, or 100 mm. In some cases, the cylindrical region of the inner tube has a diameter of less than 10, 20, 30, 40, 50, or 100 mm.

In some cases, the first or outer tube comprises a second or inner tube. In some cases, the second tube comprises an outer diameter approximately equal to an inner diameter of the first tube 110. In some cases, the centrifuge tube described herein comprises a stabilizer 168, which, at times, is provided at an end of a second tube 120. In some cases, the stabilizer is permanently coupled to an end of the second tube 120. In some cases, the stabilizer is removably or reversibly coupled to an end of the second tube. In some cases, the stabilizer is permanently coupled to an end of the first or outer tube 120. In some cases, the stabilizer is removably or reversibly coupled to an end of the first or outer tube.

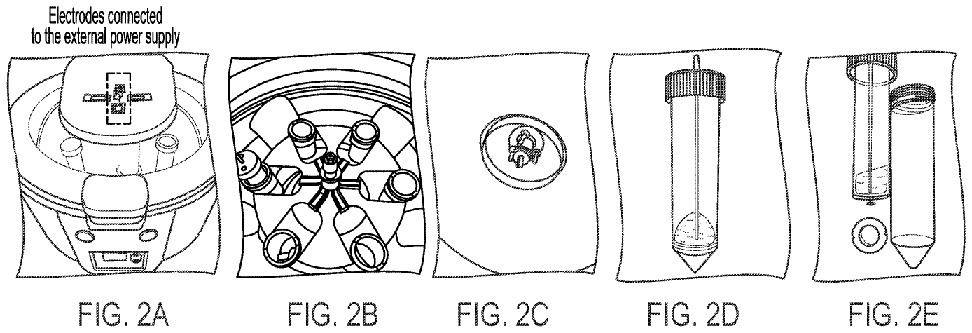

In some cases, the second tube 120 is removable from the first tube (removal of the second tube is depicted, for example, in FIG. 2E). In some cases, a cap 115 is coupled to one end of the second tube 120. In some cases, the cap 115 is coupled to an end of the second tube opposite of the stabilizer 168. In some cases, the cap 115 is permanently coupled to the second tube 120. In some cases, threading the cap 115 onto an end of the first tube 110 positions the second tube 120 within the first tube.

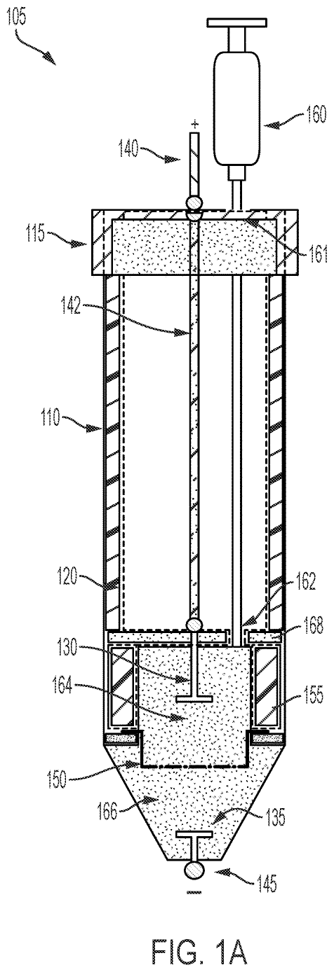

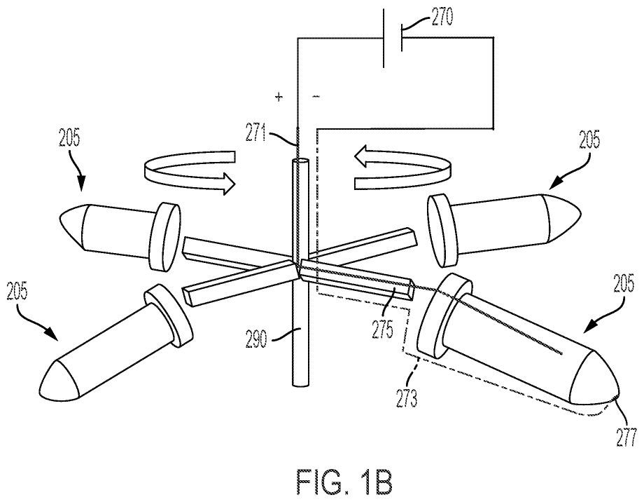

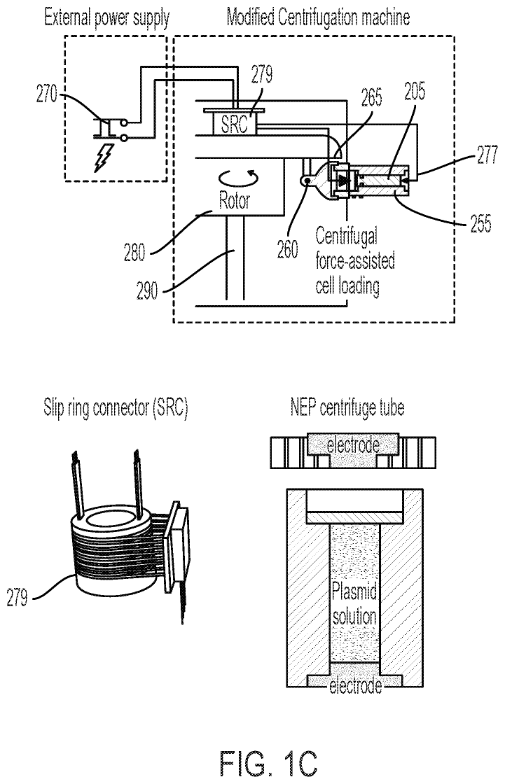

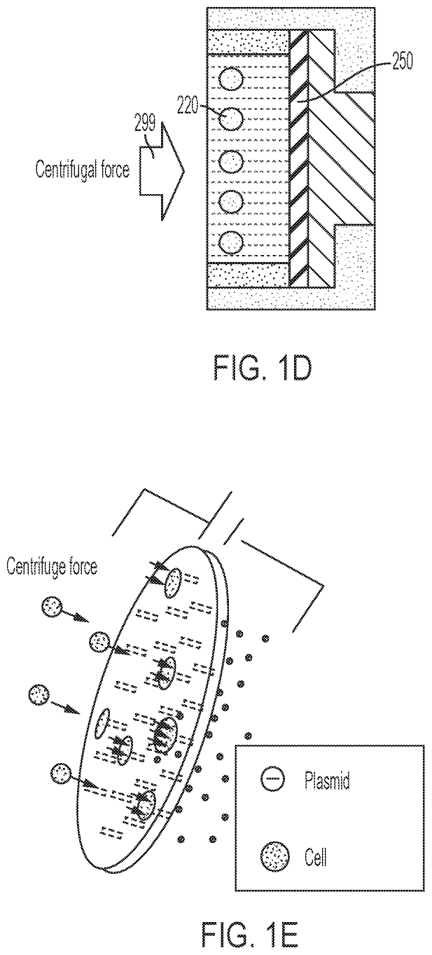

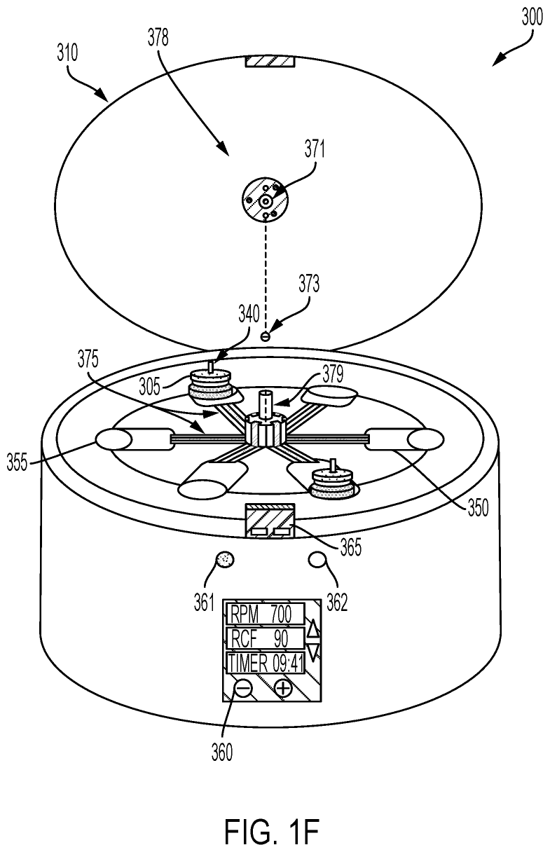

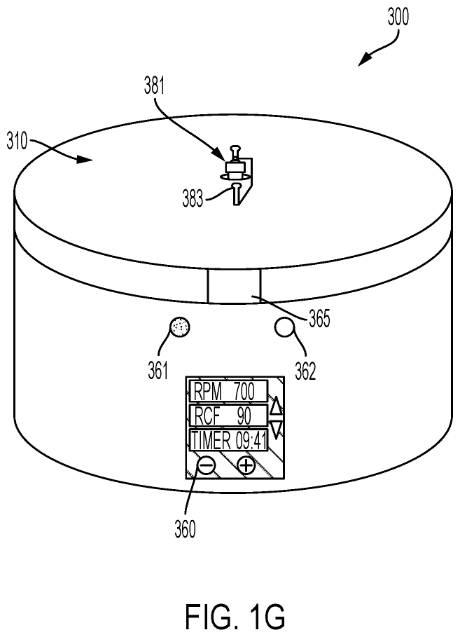

In some cases, the centrifuge tube (e.g., outer tube or first tube) described herein comprises an electroporation chip 150. In some cases, the first tube or outer tube 110 comprises an electroporation chip 150 (see FIG. 1). In some cases, the second tube or inner tube comprises an electroporation chip. In some cases, the electroporation chip is a nanopore electroporation chip; in some cases the electroporation is a micro-pore electroporation chip. In some cases, the electroporation chip 150 is disposed within the first tube 110. In some cases, the electroporation chip 150 is disposed beneath the second tube 120. In some cases, the centrifuge tube 105 is configured to receive a plurality of cells for electroporation on an electroporation chip 150 provided in the centrifuge tube. In some cases, the centrifuge tube 105 is received by a centrifuge, as disclosed herein. In some cases, the centrifuge provides centrifugal forces to press the plurality of cells against the electroporation chip 150.

The electroporation chip 150 described herein can be made of any non-conductive material. In some cases, the electroporation chip 150 is made of glass. In some cases, the electroporation chip 150 is made of ceramic. In some cases, the electroporation chip 150 is made of rubber. In some cases, the electroporation chip 150 is made of plastic. In some cases, the electroporation chip 150 is made of fiberglass. In some cases, the electroporation chip 150 is made of quartz. In some cases, the electroporation chip 150 is a silicon electroporation chip. In some specific case, the electroporation chip is a silicon wafer-based chip. In some cases, the electroporation chip comprises a silicon dioxide coating. In some cases, the electroporation chip comprises a laminin-coated filter. In some cases, the electroporation chip 150 comprises a polyethylene terepthalate (PET) membrane. In some cases, the electroporation chip 150 comprises a Transwell® insert. In some specific cases, the electroporation chip 150 comprises a polycarbonate or polyester Transwell® insert. In some specific cases, the electroporation chip 150 comprises a 12 mm Transwell® insert. In some cases, the electroporation chip 150 comprises a smooth surface. In some cases, the electroporation chip 150 comprises a rough surface.

In some cases, the electroporation chip 150 comprises a plurality of pores forming an array. In some cases, the pores are nanopores. In some cases, the pores are micropores. In some cases, the pores comprise entirely nanopores, entirely micropores, or a mixture of nanopores and micropores. In some cases, spacing between the pores is uniform. In some cases, spacing between the pores is non-uniform. In some cases, the pores have a uniform diameter. In some cases, the pores have a diameter of 50 nanometers (nm) to 10 micrometers (μm). In some cases, the pores have a diameter of about 50 nm to about 100 nm, about 50 nm to about 200 nm, about 50 nm to about 400 nm, about 50 nm to about 500 nm, about 50 nm to about 700 nm, about 50 nm to about 1,000 nm, about 50 nm to about 5,000 nm, about 50 nm to about 10,000 nm, about 100 nm to about 200 nm, about 100 nm to about 400 nm, about 100 nm to about 500 nm, about 100 nm to about 700 nm, about 100 nm to about 1,000 nm, about 100 nm to about 5,000 nm, about 100 nm to about 10,000 nm, about 200 nm to about 400 nm, about 200 nm to about 500 nm, about 200 nm to about 700 nm, about 200 nm to about 1,000 nm, about 200 nm to about 5,000 nm, about 200 nm to about 10,000 nm, about 400 nm to about 500 nm, about 400 nm to about 700 nm, about 400 nm to about 1,000 nm, about 400 nm to about 5,000 nm, about 400 nm to about 10,000 nm, about 500 nm to about 700 nm, about 500 nm to about 1,000 nm, about 500 nm to about 5,000 nm, about 500 nm to about 10,000 nm, about 700 nm to about 1,000 nm, about 700 nm to about 5,000 nm, about 700 nm to about 10,000 nm, about 700 nm to about 15,000 nm, about 1,000 nm to about 1,500 nm, about 1,000 nm to about 5,000 nm, about 1,000 nm to about 10,000 nm. In some cases, the pores have a diameter of about 50 nm, about 100 nm, about 200 nm, about 400 nm, about 500 nm, about 700 nm, about 1,000 nm, about 5,000 nm, about 10,000 nm, or including increments therein. In some cases, the pores have a diameter of at least about 50 nm, at least about 100 nm, at least about 150 nm, at least about 200 nm, at least about 250 nm, at least about 300 nm, at least about 350 nm, at least about 400 nm, at least about 450 nm, at least about 500 nm, at least about 550 nm, at least about 600 nm, at least about 650 nm, at least about 700 nm, at least about 750 nm, at least about 800 nm, at least about 850 nm, at least about 900 nm, at least about 950 nm, at least about 1,000 nm, at least about 1500 nm, at least about 2000 nm, at least about 2500 nm, at least about 3000 nm, at least about 3500 nm, at least about 4000 nm, at least about 4500 nm, at least about 5,000 nm, at least about 5500 nm, at least about 6000 nm, at least about 6500 nm, at least about 7000 nm, at least about 8000 nm, at least about 9000 nm, at least about 10,000 nm. In some cases, the pores have a diameter of at most about 50 nm, at most about 100 nm, at most about 150 nm, at most about 200 nm, at most about 250 nm, at most about 300 nm, at most about 350 nm, at most about 400 nm, at most about 450 nm, at most about 500 nm, at most about 550 nm, at most about 600 nm, at most about 650 nm, at most about 700 nm, at most about 750 nm, at most about 800 nm, at most about 850 nm, at most about 900 nm, at most about 950 nm, at most about 1,000 nm, at most about 1500 nm, at most about 2000 nm, at most about 2500 nm, at most about 3000 nm, at most about 3500 nm, at most about 4000 nm, at most about 4500 nm, at most about 5,000 nm, at most about 5500 nm, at most about 6000 nm, at most about 6500 nm, at most about 7000 nm, at most about 8000 nm, at most about 9000 nm, at most about 10,000 nm.

In some cases, the pore or channel depth is about 0.5 μm to about 20 μm, including increments therein. In some cases, the pore or channel depth is about 0.5 μm to about 1 μm, about 0.5 μm to about 5 μm, about 0.5 μm to about 10 μm, about 0.5 μm to about 15 μm, about 0.5 μm to about 20 μm, about 1 μm to about 5 μm, about 1 μm to about 10 μm, about 1 μm to about 15 μm, about 1 μm to about 20 μm, about 5 μm to about 10 μm, about 5 μm to about 15 μm, about 5 μm to about 20 μm, about 10 μm to about 15 μm, about 10 μm to about 20 μm, about 15 μm to about 20 μm. In some cases, the pore or channel depth is about 0.5 μm, about 1 μm, about 5 μm, about 10 μm, about 15 μm, about 20 μm. In some cases, the pore or channel depth is at least about 0.5 μm, at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 15 μm, at least about 20 μm. In some cases, the pore or channel depth is at most about 0.5 μm, at most about 1 μm, at most about 2 μm, at most about 3 μm, at most about 4 μm, at most about 5 μm, at most about 6 μm, at most about 7 μm, at most about 8 μm, at most about 9 μm, at most about 10 μm. at most about 15 μm, or at most about 20 μm.

In some cases, the electroporation chip 150 comprises a plurality of pores with an average pore density of about 0.001 pore/μm ² to about 10 pores/μm ². In some cases, the electroporation chip 150 comprises a plurality of pores with an average pore density of at least about 0.001 pore/μm ², at least about 0.005 pore/μm², at least about 0.01 pore/μm², at least about 0.02 pore/μm², at least about 0.03 pore/μm², at least about 0.04 pore/μm², at least about 0.05 pore/μm², at least about 0.06 pore/μm², at least about 0.07 pore/μm², at least about 0.08 pore/μm², at least about 0.09 pore/μm², at least about 0.1 pore/μm², at least about 0.2 pore/μm², at least about 0.3 pore/μm², at least about 0.4 pore/μm², at least about 0.5 pore/μm², at least about 0.6 pore/μm², at least about 0.7 pore/μm², at least about 0.8 pore/μm², at least about 0.9 pore/μm², at least about 1 pore/μm², at least about 2 pores/μm², at least about 3 pores/μm², at least about 4 pores/μm², at least about 5 pores/μm², at least about 6 pores/μm², at least about 7 pores/μm², at least about 8 pores/μm², at least about 9 pores/μm², or at least about 10 pores/μm². In some cases, the electroporation chip 150 comprises a plurality of pores with an average pore density of at most about 0.001 pore/μm², at most about 0.005 pore/μm², at most about 0.01 pore/μm², at most about 0.02 pore/μm², at most about 0.03 pore/μm², at most about 0.04 pore/μm², at most about 0.05 pore/μm², at most about 0.06 pore/μm², at most about 0.07 pore/μm², at most about 0.08 pore/μm², at most about 0.09 pore/μm², at most about 0.1 pore/μm², at most about 0.2 pore/μm², at most about 0.3 pore/μm², at most about 0.4 pore/μm², at most about 0.5 pore/μm², at most about 0.6 pore/μm², at most about 0.7 pore/μm², at most about 0.8 pore/μm², at most about 0.9 pore/μm², at most about 1 pore/μm², at most about 2 pores/μm², at most about 3 pores/μm², at most about 4 pores/μm², at most about 5 pores/μm², at most about 6 pores/μm², at most about 7 pores/μm², at most about 8 pores/μm², at most about 9 pores/μm², or at most about 10 pores/μm².

In some cases, a cell chamber wall 155 and the electroporation chip 150 form an integral component which is removable from the first tube 110 (as depicted in FIG. 2E). In some cases, the outer circumference of the cell chamber wall 155 is approximately equal to the inner diameter of the first tube 110. In some cases, the outer circumference of the cell chamber wall provides a friction fit when disposed with the first tube 110. In some cases, the outer circumference of the cell chamber wall 155 abuts the end of the first tube where the first tube begins to taper to form a conical end. In some cases, the inner circumference of the cell chamber wall 155 comprises a diameter approximately equal to the diameter of the electroporation chip 150. In some cases, the electroporation chip 150 and the cell chamber wall 155 form an integral component. In some cases, the electroporation chip and the cell chamber wall are provided as a permeable cell culture insert.

In some cases, the centrifuge tube described herein comprises a cell chamber 164. In some cases, the cell chamber receives the plurality of cells to be subjected to electroporation. In some cases, the plurality of cells is provided in a suspension. In some cases, the cell chamber 164 is formed by a side of a stabilizer 168 and a first side of the electroporation chip 150. In some cases, the stabilizer 168 comprises an outer diameter approximately equal to an inner diameter of the first tube 110, such that a friction fit is formed between the stabilizer 168 and the first tube 110. In some cases, the fit between the stabilizer 168 and the first tube 110 provides a liquid-tight seal. In some cases, the fit between the stabilizer 168 and the first tube 110 provides an air-tight seal.

In some cases, the centrifuge tube described herein comprises a transfection reagent chamber 166. In some cases, the distal portion of the first tube 110 comprises the transfection reagent chamber. In some cases, the transfection reagent chamber is on a second side of the electroporation chip. In some cases, the transfection reagent chamber is located between the second side of the electroporation chip and the closed end of the first tube 110.

In some cases, transfection reagents are disposed within the transfection reagent chamber 166. In some cases, the transfection reagents have small molecular weight. In some cases, the transfection reagents have large molecular weight. In some cases, the molecular weight of the transfection reagents is about 100 g/mol to about 1000 g/mol. In some cases, the molecular weight of the transfection reagents is about 1000 g/mol to about 2000 g/mol. In some cases, the molecular weight of the transfection reagents is about 2000 g/mol to about 3000 g/mol. In some cases, the molecular weight of the transfection reagents is about 3000 g/mol to about 4000 g/mol. In some cases, the molecular weight of the transfection reagents is about 4000 g/mol to about 5000 g/mol. In some cases, the molecular weight of the transfection reagents is about 5000 g/mol to about 7500 g/mol. In some cases, the molecular weight of the transfection reagents is about 7500 g/mol to about 10000 g/mol. In some cases, the transfection reagents comprise DNAs, RNAs, proteins, other charged biomolecules, and/or charged molecules. In some specific cases, the transfection reagents comprise plasmid DNAs. In some specific cases, the transfection reagents comprise siRNAs. In some specific cases, the transfection reagents comprise mRNA, encapsulated in nanolipid particles or not. In some specific cases, the transfection reagents comprise miRNAs. In some specific cases, the transfection reagents comprise shRNAs. In some specific cases, the transfection reagents comprise a small-molecule drug. In some specific cases, the transfection reagents comprise polypeptides. In some specific cases, the transfection reagents comprise antibodies. In some specific cases, the transfection reagents comprise a combination of the above-described reagents.

In some cases, the centrifuge tube described herein is configured to conduct electroporation on a plurality of cells disposed within the centrifuge tube. In some cases, an electroporation chip 150 is disposed within the centrifuge tube. In some cases, the electroporation chip is sandwiched between a cell chamber 164 and a transfection reagent chamber 166. In some cases, the electroporation chip 164 provides a physical barrier between the transfection reagent chamber 166 and the cell chamber 164.

In some cases, the centrifuge tube described herein comprises two electrodes 130 and 135 for providing an electric field for cell electroporation or transfection. In some cases, a circuit comprises the first electrode 130 and the second electrode 135 and a power source. In some cases, the power source provides an electric potential between the first electrode 130 and the second electrode 135 to produce an electric field across electroporation chip 150. In some cases, the first electrode 130 is a positive electrode. In some cases, the second electrode 135 is a negative electrode. In some cases, the first electrode 130 is a negative electrode. In some cases, the second electrode 135 is a positive electrode. In some cases, the first electrode 130 is an anode. In some cases, the second electrode 135 is a cathode. In some cases, the first electrode 130 is a cathode. In some cases, the second electrode 135 is an anode. In some cases, a first electrode contact 140 is provided to couple the first electrode 130 via a first electrode wire 142, to the power source. In some cases, a second electrode contact 145 is provided to couple the second electrode 135 to the power source.

In some cases, a first electrode 130 is provided on a first side of the electroporation chip. In some cases, a first electrode 130 is disposed within the cell chamber 164 of the centrifuge tube. In some cases, the first electrode 130 is electrically coupled to a first electrode contact 140 via an electrode wire 142. In some cases, the first electrode contact 140 is provided on an exterior of the first tube 110. In some cases, the first electrode contact 140 provides an electrical connection point for connecting to an electrical circuit external to the centrifuge tube. In some cases, the first electrode contact 140 provides an electric potential to the first electrode 130 when connected to an electric power source. In some cases, the first electrode wire 142 runs through the second tube 120 from the first electrode contact 140 to the first electrode 130. In some cases, an aperture is provided through the cap 115 to allow the electrode wire to pass through a wall of the cap. In some cases, an aperture is provided through the stabilizer to allow an electrical connection of the electrode wire 142 of a first side of the stabilizer to the first electrode 130 on a second side of the stabilizer. In some cases, the cap 115, the second tube 120, the first electrode contact 140, electrode wire 142, and first electrode 130 are coupled, such that coupling of the cap 115 to the first tube 110 positions the first electrode 130 within the cell chamber 164. In some cases, the first electrode contact is provided along a center axis of the centrifuge tube 105.

In some cases, a second electrode 135 is provided within the transfection reagent chamber 166. In some cases, a second electrode 135 is provided on a second side of the electroporation chip. In some cases, the second electrode 135 is coupled to the first tube 110 at the conical end. In some cases, the second electrode 135 is in electrical communication with a second electrode contact 145. In some cases, the second electrode contact 145 provides an electric potential to the second electrode 135 when connected to an electric power source. In some cases, the second electrode contact is provided along a center axis of the centrifuge tube 105. In some cases, second electrode contact 145 is provided at the exterior of the first tube 110. In some cases, second electrode contact 145 is proximal to the closed end of the first tube 110. In some cases, the second electrode contact 145 is provided on a side of the first tube 110 opposite side of the first electrode contact 140. In some cases, the connection between the second electrode 135 and the second electrode contact 145 runs through an outer wall of the end of the first tube 110.

The first electrode contact 140 and the second electrode contact 145 may be coupled to an electrical circuit, such that an electric potential provided between the first electrode 130 and the second electrode 135 and across the electroporation chip 150. In some cases, first electrode 130 and the second electrode 135 are configured to create an electric field across the electroporation chip 150. In some cases, application of an electric field across the electroporation chip increases permeability of cell membranes present within the cell chamber 164 for transfection by reagents provided within the transfection reagent chamber 166. In some cases, the first electrode 130 and second electrode 135 comprise platinum electrodes. In some cases, the first electrode 130 and second electrode 135 comprise copper, graphite, titanium, brass, silver, gold, or other suitable materials. In some cases, the first electrode 130 is configured as a cathode. In some cases, the second electrode 135 is configure as an anode.

In some cases, the centrifuge tube described herein further comprises a structure that extend the exterior of the centrifuge tube and the cell chamber. In some specific cases, the centrifuge tube described herein further comprises a syringe 160. In other specific cases, the centrifuge tube described herein further comprises a serological pipette.

In some cases, the centrifuge tube described herein further comprises a stabilizer 168 that can serve to hold the syringe 160 described herein and the first electrode wire 142 or effectively the first electrode 140 in place during the centrifugation. In some cases, the stabilizer described herein comprises an electrode wire aperture such that the first electrode wire 142 passes through the electrode wire aperture to provide an electrical connection from the first electrode 130 to the first electrode contact 140. In some specific cases, the stabilizer holds the first electrode wire 142 in place. In some cases, the stabilizer described herein comprises a syringe through hole 162, for receiving the syringe 160 to provide cells to the cell chamber 164. In some cases, a friction fit is provided between the stabilizer 168 and the first tube 110. In some specific cases, the friction fit facilitates the retention of the second tube 120 within the first tube 110. In some cases, the stabilizer 168 provides a liquid tight seal of the cell chamber 164. In some cases, the stabilizer 168 provides an air-tight seal of the cell chamber.

In some cases, the centrifuge tube described herein further comprises a cap. In some cases, the cap described herein is removable. In some cases, the cap described herein comprises an aperture from where the first electrode wire 142 passes through. In some cases, the cap described herein comprises a syringe aperture for receiving a syringe 160 to provide a plurality of cells to the cell chamber 164. In some specific cases, the syringe aperture described herein is positioned off a center axis of the cap.

In some cases, the cells are injected into the cell chamber via a syringe 160. In some cases, the cap 115 comprises a syringe aperture 161 to allow a syringe 160 to pass through the cap and into the second tube 120. In some cases, the stabilizer 168 comprises a syringe through hole 162 to allow a syringe 160 to pass through the stabilizer and into the cell chamber 164. In some cases, the first syringe aperture and the second syringe aperture comprise one-way seals to prevent unwanted expulsion of cells in a suspension from the cell chamber 164. In some cases, the syringe is used to withdraw cells, which have undergone electroporation, from the cell chamber 164. In some cases, withdrawn cells are then incubated.

### II. Centrifuges

Described herein are centrifuges for providing an electrical current to a suspension within one or more centrifuge tubes, wherein the centrifuges comprise: a rotor comprising a hub, at least one tube holder connected to the hub, and a circuit for providing an electric current and voltage through at least one centrifuge tube or each centrifuge tube of the one or more centrifuge tubes.

In some cases, the centrifuge described herein comprises a rotor 280. In some cases. The rotor 280 comprises a hub. In some cases, the rotor 280 described herein is rotated to produce a relative centrifugal force (RCF) of about 1 to 3000 g. In some cases, the centrifuge rotates at about 1 g to about 50 g, about 1 g to about 100 g, about 1 g to about 300 g, about 1 g to about 500 g, about 1 g to about 700 g, about 1 g to about 1,000 g, about 1 g to about 1,500 g, about 1 g to about 2,000 g, about 1 g to about 2,500 g, about 1 g to about 3,000 g, about 50 g to about 100 g, about 50 g to about 300 g, about 50 g to about 500 g, about 50 g to about 700 g, about 50 g to about 1,000 g, about 50 g to about 1,500 g, about 50 g to about 2,000 g, about 50 g to about 2,500 g, about 50 g to about 3,000 g, about 100 g to about 300 g, about 100 g to about 500 g, about 100 g to about 700 g, about 100 g to about 1,000 g, about 100 g to about 1,500 g, about 100 g to about 2,000 g, about 100 g to about 2,500 g, about 100 g to about 3,000 g, about 300 g to about 500 g, about 300 g to about 700 g, about 300 g to about 1,000 g, about 300 g to about 1,500 g, about 300 g to about 2,000 g, about 300 g to about 2,500 g, about 300 g to about 3,000 g, about 500 g to about 700 g, about 500 g to about 1,000 g, about 500 g to about 1,500 g, about 500 g to about 2,000 g, about 500 g to about 2,500 g, about 500 g to about 3,000 g, about 700 g to about 1,000 g, about 700 g to about 1,500 g, about 700 g to about 2,000 g, about 700 g to about 2,500 g, about 700 g to about 3,000 g, about 1,000 g to about 1,500 g, about 1,000 g to about 2,000 g, about 1,000 g to about 2,500 g, about 1,000 g to about 3,000 g, or about 1,500 g to about 2,000 g, about 1,500 g to about 2,500 g, about 1,500 g to about 3,000 g, about 2,000 g to about 2,500 g, about 2,000 g to about 3,000 g, or about 2,500 g to about 3,000 g. In some cases, the centrifuge rotates at about 1 g, about 50 g, about 100 g, about 300 g, about 500 g, about 700 g, about 1,000 g, about 1,500 g, or about 2,000 g. In some cases, the centrifuge rotates at least about 1 g, about 50 g, about 100 g, about 300 g, about 500 g, about 700 g, about 1,000 g, or about 1,500 g. In some cases, the centrifuge rotates at most about 50 g, about 100 g, about 300 g, about 500 g, about 700 g, about 1,000 g, about 1,500 g, about 2,000 g, about 2,500 g, about 3,000 g.

With reference to FIGS. 1B and 1C, an internal depiction of centrifuge tubes 205 loaded into a centrifuge is depicted. In some cases, the centrifuge tubes 205 are loaded into tube holders 255. In some cases, the centrifuge tubes are configured for electroporation of cells, as disclosed herein. In some cases, the tube holders 255 are pivoting tube holders connected to a rotor 280 of the centrifuge via a hinge or pivotable coupling 260, such that the tube holders 255, and centrifuge tubes 205 provided in the tube holders, rotate under the influence of a centrifugal force provided by the centrifuge. In some cases, the tube holders 255 are pivotably coupled to the hub such that the tube holders 255 rotate towards a horizontal orientation or towards about a horizontal orientation as the rotor is rotated. In some cases, the tube holders 255 are pivotably coupled to the hub such that the tube holders 255 rotate towards an angled orientation as the rotor is rotated. In some cases, the centrifuge tubes 205 is rotatable around the hub. In some cases, the centrifuge tubes 205 pivots under influence of a centrifugal force applied by the rotor 280.

In some cases, the centrifuge described herein comprises a swing-bucket rotor. In the cases where a swing-bucket rotor is used, the tube holders 255 are pivotably coupled to the hub such that the tube holders 255 rotate towards a horizontal orientation or towards about a horizontal orientation as the rotor is rotated. In some cases, the centrifuge described herein comprises a fixed-angel rotor. In the cases where a fixed-angel rotor is used, the tube holders 255 are pivotably coupled to the hub such that the tube holders 255 rotate towards an angled orientation as the rotor is rotated.

In some cases, a circuit is provided through the centrifuge to provide an electrical connection from a power source 270 to electrodes of a centrifuge tube 205 configured for electroporation of cells, as disclosed herein. In some cases, the power source 270 is external to the centrifuge 300. In some cases, the power source 270 is within the centrifuge 300. In some cases, the electroporation centrifuge system is configured, such that the electrical connection from the power source 270 to electrodes of the centrifuge tubes 205 is only established when the centrifuge tubes are provided at the desired angle after pivoting under influence of the centrifugal force provided by the centrifuge. In some cases, a first electrode contact (140 as depicted in FIG. 1A) extending from the centrifuge tube 205 only contacts a first electrical contact 275 of a circuit when the centrifuge tube is provided at the desired angle due to the centrifugal forces acting on the tube holder and tube during the rotation of the centrifuge. In some specific cases, a first electrode contact 140 only contacts a first electrical contact 275 of a circuit when the centrifuge tube is about perpendicular or exactly perpendicular to the center axis of the centrifuge rotor during the rotation of the centrifuge. In some specific cases, a first electrode contact 140 always contacts a first electrical contact 275 of a circuit, and a connector with switch is installed between the power source and the circuit. In some cases, a second electrode contact (145 as depicted in FIG. 1A) is provided in electrical communication with a power source by a second electrical contact 277 of a circuit. In some cases, the second electrical contact 277 is provided by a chassis, including a rotation frame, of the centrifuge the electric connector on the outer chassis surface. In some cases, when the tube is placed into the centrifuge tube holder, the second electrical contact (145 as depicted in FIG. 1A) contacts the rotation frame. In some cases, conductive strip is placed from at least one centrifuge tube or each centrifuge tube location to a central screw location. In some cases, a holder with conductive strips on the outer surface and an electrical socket on the top of the holder is tightly mounted onto a central screw. In some cases, lead wires 271, 273 provide an electrical communication of the electrical contacts 275, 277 of the circuit.

In some cases, the centrifuge comprises a rotatable electrical coupling 279 to maintain an electrical communication between a power source 270 and the circuit within the centrifuge during the rotation of the centrifugation. In some specific cases, the rotatable electrical coupling 279 comprises a slip ring connector, which rotates together with the rotor during the rotation of the centrifugation.

With reference to FIG. 1D, a representation of a plurality of cells 220 within a suspension provided in a centrifuge tube are depicted under a centrifugal force 299, according to some cases. In some cases, under a centrifugal force 299, the plurality of cells 220 are pushed toward and against a surface the electroporation chip 250 within the centrifuge tube to provide efficient electroporation of the cells.

In some cases, the tube holders 255 pivot to an angle about perpendicular or exactly perpendicular to the center axis 290 of the centrifuge rotor 280. In some cases, a stop wall, or barrier 265 is provided to stop rotation of a tube holder at the desired angle. In some cases, the angle about perpendicular to the center axis 290 corresponds to an angle which is about perpendicular to the force of acceleration due to gravity.

In some cases, a connector provides electrical communication between the power source and the circuit described herein. With reference to FIGS. 1F and 1G, a centrifuge 300 configured for providing an electrical current to one or more centrifuge tubes 350 is depicted, according to some cases. In some cases, the centrifuge 300 comprises a centrifuge cover 310. In some cases, the centrifuge 300 comprises a rotatable electrical coupling 379 to mate with a power source connector 378 when the cover 310 is closed. In some cases, an external power source connects to a plug on the outer surface of the cover. In some cases, the plug comprises a first outer terminal 381 and a second outer terminal 383. In some cases, the first outer terminal 381 is a positive terminal, and the second outer terminal 383 is a negative terminal. In some cases, the first terminal 381 is in electrical communication with a first inner terminal 371 of the power source connector 378 provided on the inside surface of the cover 310. In some cases, the second terminal 383 is in electrical communication with a second inner terminal 373 of the power source connector 378 provided on the inside surface of the cover 310.

In some cases, when the lid is closed, the rotatable electrical coupling 379 mates with a power source connector such that the input (e.g. negative input signal) from the power source connected to the second outer terminal 383 to transferred through the second inner terminal 373 and to one or more second electrical contacts (e.g. second electrical contact 277 depicted in FIGS. 1F and 1G) provided on tube holders 355. In some cases, the second electrical contacts provided on tube holders 355 make contact with second electrode contacts (e.g. second electrode contact 145 as depicted FIG. 1) of centrifuge tubes 305 configured for electroporation when the tubes are placed within the tube holders.

In some cases, when the lid is closed, the rotatable electrical coupling 379 mates with a power source connector such that the input (e.g. positive input signal) from the power source connected to the first outer terminal 381 to transferred through the second inner terminal 371 and to conductive strips 375 provided on a rotor 350 of the centrifuge 300. In some cases, first electrode contacts 340 of a centrifuge tubes 305 placed in the tube holders 355 contact the conductive strips 375 as the tube holders pivot under the influence of centrifugal forces created by the rotation of the centrifuge. In some cases, contact of a first electrode contact 340 to a conductive strip 375 completes the circuit, provided an electric potential between the first and second electrodes (e.g. 130 and 135 depicted in FIG. 1) of the centrifuge tube 350 and provides an electric field across an electroporation chip within the tube to electroporate cells. In some cases, the first electrode contacts 340 further comprise a spring to facilitate contact with the conductive strips. In some cases, conductive silver paste is placed in the socket and the bottom of the centrifuge tube to improve electrical connection with low resistance.

In some cases, a current is applied to a circuit comprising the first electrode 130 and the second electrode 135 to produce an electric field. In some cases, the current applied to the first electrode 130 and the second electrode 135 at a voltage/distance between two electrodes of 0.5 V/cm to 1000V/cm, including increments therein. In some cases, the voltage is applied at as a pulse. In some cases, the pulse length is 1 to 50 milliseconds (ms) including increments therein. In some cases, the voltage is applied as a series of pulses. In some cases, the series of pulses comprises 1 to 100 pulses. In some cases, the pulses are applied as a square wave signal. In some cases, the duration between pulses (no voltage applied, pulse interval) is approximately equal to the selected pulse duration. For example, if a pulse length of 10 ms is utilized, the pulse cycle includes a voltage application for 10 ms followed by a period of no voltage application for 10 ms. In other cases, the pulse interval is longer than the selected pulse duration. For example, if a pulse length of 10 ms is utilized, the pulse cycle includes a voltage application for 10 ms followed by a period of no voltage application for 100 ms. In other cases, the pulse interval is shorter than the selected pulse duration.

In some cases, the applied voltage/distance between the two electrodes is about 0.5 V/cm to about 1,000 V/cm. In some cases, the applied voltage/distance between the two electrodes is about 0.5 V/cm to about 1 V/cm, about 0.5 V/cm to about 100 V/cm, about 0.5 V/cm to about 300 V/cm, about 0.5 V/cm to about 500 V/cm, about 0.5 V/cm to about 800 V/cm, about 0.5 V/cm to about 1,000 V/cm, about 1 V/cm to about 100 V/cm, about 1 V/cm to about 300 V/cm, about 1 V/cm to about 500 V/cm, about 1 V/cm to about 800 V/cm, about 1 V/cm to about 1,000 V/cm, about 100 V/cm to about 300 V/cm, about 100 V/cm to about 500 V/cm, about 100 V/cm to about 800 V/cm, about 100 V/cm to about 1,000 V/cm, about 300 V/cm to about 500 V/cm, about 300 V/cm to about 800 V/cm, about 300 V/cm to about 1,000 V/cm, about 500 V/cm to about 800 V/cm, about 500 V/cm to about 1,000 V/cm, or about 800 V/cm to about 1,000 V/cm. In some cases, the applied voltage/distance between the two electrodes is about 0.5 V/cm, about 1 V/cm, about 100 V/cm, about 300 V/cm, about 500 V/cm, about 800 V/cm, or about 1,000 V/cm. In some cases, the applied voltage is at least about 0.5 V/cm, about 1 V/cm, about 100 V/cm, about 300 V/cm, about 500 V/cm, or about 800 V/cm. In some cases, the applied voltage/distance between the two electrodes is at most about 1 V/cm, about 100 V/cm, about 300 V/cm, about 500 V/cm, about 800 V/cm, or about 1,000 V/cm.

In some cases, a pulse length is about 1 ms to about 50 ms. In some cases, a pulse duration is about 1 ms to about 5 ms, about 1 ms to about 10 ms, about 1 ms to about 15 ms, about 1 ms to about 20 ms, about 1 ms to about 30 ms, about 1 ms to about 50 ms, about 5 ms to about 10 ms, about 5 ms to about 15 ms, about 5 ms to about 20 ms, about 5 ms to about 30 ms, about 5 ms to about 50 ms, about 10 ms to about 15 ms, about 10 ms to about 20 ms, about 10 ms to about 30 ms, about 10 ms to about 50 ms, about 15 ms to about 20 ms, about 15 ms to about 30 ms, about 15 ms to about 50 ms, about 20 ms to about 30 ms, about 20 ms to about 50 ms, or about 30 ms to about 50 ms. In some cases, a pulse duration is about 1 ms, about 5 ms, about 10 ms, about 15 ms, about 20 ms, about 30 ms, or about 50 ms. In some cases, a pulse duration is at least about 1 ms, about 5 ms, about 10 ms, about 15 ms, about 20 ms, or about 30 ms. In some cases, a pulse duration is at most about 5 ms, about 10 ms, about 15 ms, about 20 ms, about 30 ms, or about 50 ms.

In some cases, a voltage cycle or series comprises about 1 pulse to about 200 pulses. In some cases, a voltage cycle comprises about 1 pulse to about 10 pulses, about 1 pulse to about 30 pulses, about 1 pulse to about 50 pulses, about 1 pulse to about 70 pulses, about 1 pulse to about 100 pulses, about 1 pulse to about 200 pulses, about 10 pulses to about 30 pulses, about 10 pulses to about 50 pulses, about 10 pulses to about 70 pulses, about 10 pulses to about 100 pulses, about 10 pulses to about 200 pulses, about 30 pulses to about 50 pulses, about 30 pulses to about 70 pulses, about 30 pulses to about 100 pulses, about 30 pulses to about 200 pulses, about 50 pulses to about 70 pulses, about 50 pulses to about 100 pulses, about 50 pulses to about 200 pulses, about 70 pulses to about 100 pulses, about 70 pulses to about 200 pulses, or about 100 pulses to about 200 pulses. In some cases, a voltage cycle comprises about 1 pulse, about 10 pulses, about 30 pulses, about 50 pulses, about 70 pulses, about 100 pulses, or about 200 pulses. In some cases, a voltage cycle comprises at least about 1 pulse, about 10 pulses, about 30 pulses, about 50 pulses, about 70 pulses, or about 100 pulses. In some cases, a voltage cycle comprises at most about 10 pulses, about 30 pulses, about 50 pulses, about 70 pulses, about 100 pulses, or about 200 pulses.

In some cases, the pulse interval is about 1 ms to about 200 ms. In some cases, the pulse interval is about 1 ms to about 5 ms, about 1 ms to about 10 ms, about 1 ms to about 20 ms, about 1 ms to about 30 ms, about 1 ms to about 40 ms, about 1 ms to about 50 ms, about 1 ms to about 100 ms, about 1 ms to about 200 ms, about 5 ms to about 10 ms, about 5 ms to about 20 ms, about 5 ms to about 30 ms, about 5 ms to about 40 ms, about 5 ms to about 50 ms, about 5 ms to about 100 ms, about 5 ms to about 200 ms, about 10 ms to about 20 ms, about 10 ms to about 30 ms, about 10 ms to about 40 ms, about 10 ms to about 50 ms, about 10 ms to about 100 ms, about 10 ms to about 200 ms, about 20 ms to about 30 ms, about 20 ms to about 40 ms, about 20 ms to about 50 ms, about 20 ms to about 100 ms, about 20 ms to about 200 ms, about 30 ms to about 40 ms, about 30 ms to about 50 ms, about 30 ms to about 100 ms, about 30 ms to about 200 ms, about 40 ms to about 50 ms, about 40 ms to about 100 ms, about 40 ms to about 200 ms, about 50 ms to about 100 ms, about 50 ms to about 200 ms, or about 100 ms to about 200 ms. In some cases, the pulse interval is about 1 ms, about 5 ms, about 10 ms, about 20 ms, about 30 ms, about 40 ms, about 50 ms, about 100 ms, or about 200 ms. In some cases, the pulse interval is at least about 1 ms, about 5 ms, about 10 ms, about 20 ms, about 30 ms, about 40 ms, about 50 ms, or about 100 ms. In some cases, the pulse interval is at most about 5 ms, about 10 ms, about 20 ms, about 30 ms, about 40 ms, about 50 ms, about 100 ms, or about 200 ms.

In some cases, a series or cycle of voltage pulses are applied as a waveform. In some cases, a series or cycle of voltage pulses are applied as a square waveform, a sinusoidal waveform, a triangular waveform, a sawtooth waveform, or a combination thereof. In some cases, a series or cycle of voltage pulses are applied with a constant voltage. a series or cycle of voltage pulses are applied with a constant current.

In some cases, the centrifuge 300 further comprises an operation panel 360. Operation panel 360 may allow a user to monitor the rate of rotation, the relative centrifugal force, and the duration of centrifuging. Operation panel 360 may allow a user to set the rate of rotation, the relative centrifugal force, and the duration of centrifuging.

In some cases, centrifuge 300 comprises a start button 361 to start a centrifuge cycle. In some cases, centrifuge 300 comprises a stop button 362 to manually stop a centrifuge cycle. In some cases, the centrifuge comprises a latch 365. The latch may lock the cover in a closed position upon starting the centrifuge.

### III. Electroporation Centrifuge Systems

This disclosure provides electroporation centrifuge systems, which may include one centrifuge device and one or more centrifuge tubes. Generally, the electroporation centrifuge systems comprise at least one centrifuge tube comprising: a first tube, an electroporation chip disposed within the first tube, a first electrode provided on a first side of the electroporation chip, and a second electrode provided on a second side of the electroporation chip; a centrifuge device comprising: a rotor comprising a hub; one or more tube holders coupled to the hub; and/or a circuit for providing an electric current to the one or more centrifuge tubes provided within the one or more tube holders; and/or a power source for supplying the electric current to the circuit.

In some cases, the electroporation system described herein comprises a first tube 110 configured for electroporation of a plurality of cells. In some cases, the first tube 110 comprises a 50 milliliter (50 mL) centrifuge tube. In some cases, the first tube 110, comprises a threaded circumference at a one end to receive a cap 115. In some cases, the centrifuge tube is conical at other end, opposite of the threaded circumference. In some cases, the first tube comprises plastic. In some specific cases, the first tube comprises polypropylene. In some specific cases, the first tube comprises fluoroelastomer. In some specific cases, the first tube comprises neoprene. In some specific cases, the first tube comprises nitrile. In some specific cases, the first tube comprises nylon. In some specific cases, the first tube comprises polyethylene. In some specific cases, the first tube comprises polytetrafluoroethylene. In some specific cases, the first tube comprises polyurethane. In some specific cases, the first tube comprises polyvinyl chloride. In other cases, the first tube comprises glass. In other cases, the first tube comprises ceramic. In other cases, the first tube comprises metal.

In some cases, the electroporation system described herein comprises the stabilizer 168 provided at an end of a second tube 120. In some cases, the second tube comprises an outer diameter approximately equal to an inner diameter of the first tube 110. In some cases, the stabilizer to permanently coupled to the end of the second tube 120. In some cases, the second tube 120 is removable from the first tube (removal of the second tube is depicted, for example, in FIG. 2E). In some cases, a cap 115 is coupled to one end of the second tube 120. In some cases, the cap 115 is coupled to an end of the second tube opposite of the stabilizer 168. In some cases, the cap 115 is permanently coupled to the second tube 120. In some cases, threading the cap 115 onto an end of the first tube 110 positions the second tube 120 within the first tube.

In some cases, the electroporation system described herein comprises an electroporation chip 150. In some cases, the first tube 110 comprises an electroporation chip 150 (see FIG. 1). In some cases, the electroporation chip is a nano-or micro-electroporation (N/MEP) chip. In some cases, the electroporation chip 150 is disposed within the first tube 110. In some cases, the electroporation chip 150 is disposed beneath the second tube 120. In some cases, the centrifuge tube 105 is configured to receive a plurality of cells for electroporation on an electroporation chip 150 provided in the centrifuge tube. In some cases, the centrifuge tube 105 is received by a centrifuge, as disclosed herein. In some cases, the centrifuge provides centrifugal forces to press the plurality of cells against the electroporation chip 150.

In some cases, the electroporation chip 150 provided in the centrifuge tube 105 comprises a silicon electroporation chip. In some specific case, the electroporation chip is a silicon wafer-based chip. In some cases, the electroporation chip comprises a silicon dioxide coating. In some cases, the electroporation chip comprises a laminin-coated filter. In some cases, the electroporation chip 150 comprises a polyethylene terepthalate (PET) membrane. In some cases, the electroporation chip 150 comprises a Transwell® insert. In some specific cases, the electroporation chip 150 comprises a polycarbonate or polyester Transwell® insert. In some specific cases, the electroporation chip 150 comprises a 12 mm Transwell® insert. In some cases, the electroporation chip 150 comprises a smooth surface. In some cases, the electroporation chip 150 comprises a rough surface.

In some cases, the electroporation chip 150 comprises a plurality of pores forming an array. In some cases, the pores are nanopores. In some cases, the pores are micropores. In some cases, spacing between the pores is uniform. In some cases, spacing between the pores is non-uniform. In some cases, the pores have a uniform diameter. In some cases, the pores comprise a diameter of 50 nanometers (nm) to 10 micrometers (μm). In some cases, the pores have a diameter of about 50 nm to about 100 nm, about 50 nm to about 200 nm, about 50 nm to about 400 nm, about 50 nm to about 500 nm, about 50 nm to about 700 nm, about 50 nm to about 1,000 nm, about 50 nm to about 5,000 nm, about 50 nm to about 10,000 nm, about 100 nm to about 200 nm, about 100 nm to about 400 nm, about 100 nm to about 500 nm, about 100 nm to about 700 nm, about 100 nm to about 1,000 nm, about 100 nm to about 5,000 nm, about 100 nm to about 10,000 nm, about 200 nm to about 400 nm, about 200 nm to about 500 nm, about 200 nm to about 700 nm, about 200 nm to about 1,000 nm, about 200 nm to about 5,000 nm, about 200 nm to about 10,000 nm, about 400 nm to about 500 nm, about 400 nm to about 700 nm, about 400 nm to about 1,000 nm, about 400 nm to about 5,000 nm, about 400 nm to about 10,000 nm, about 500 nm to about 700 nm, about 500 nm to about 1,000 nm, about 500 nm to about 5,000 nm, about 500 nm to about 10,000 nm, about 700 nm to about 1,000 nm, about 700 nm to about 5,000 nm, about 700 nm to about 10,000 nm, about 700 nm to about 15,000 nm, about 1,000 nm to about 1,500 nm, about 1,000 nm to about 5,000 nm, about 1,000 nm to about 10,000 nm. In some cases, the pores have a diameter of about 50 nm, about 100 nm, about 200 nm, about 400 nm, about 500 nm, about 700 nm, about 1,000 nm, about 5,000 nm, about 10,000 nm, or including increments therein. In some cases, the pores have a diameter of at least about 50 nm, at least about 100 nm, at least about 150 nm, at least about 200 nm, at least about 250 nm, at least about 300 nm, at least about 350 nm, at least about 400 nm, at least about 450 nm, at least about 500 nm, at least about 550 nm, at least about 600 nm, at least about 650 nm, at least about 700 nm, at least about 750 nm, at least about 800 nm, at least about 850 nm, at least about 900 nm, at least about 950 nm, at least about 1,000 nm, at least about 1500 nm, at least about 2000 nm, at least about 2500 nm, at least about 3000 nm, at least about 3500 nm, at least about 4000 nm, at least about 4500 nm, at least about 5,000 nm, at least about 5500 nm, at least about 6000 nm, at least about 6500 nm, at least about 7000 nm, at least about 8000 nm, at least about 9000 nm, at least about 10,000 nm. In some cases, the pores have a diameter of at most about 50 nm, at most about 100 nm, at most about 150 nm, at most about 200 nm, at most about 250 nm, at most about 300 nm, at most about 350 nm, at most about 400 nm, at most about 450 nm, at most about 500 nm, at most about 550 nm, at most about 600 nm, at most about 650 nm, at most about 700 nm, at most about 750 nm, at most about 800 nm, at most about 850 nm, at most about 900 nm, at most about 950 nm, at most about 1,000 nm, at most about 1500 nm, at most about 2000 nm, at most about 2500 nm, at most about 3000 nm, at most about 3500 nm, at most about 4000 nm, at most about 4500 nm, at most about 5,000 nm, at most about 5500 nm, at most about 6000 nm, at most about 6500 nm, at most about 7000 nm, at most about 8000 nm, at most about 9000 nm, at most about 10,000 nm.

In some cases, the pore or channel depth is about 0.5 μm to about 20 μm, including increments therein. In some cases, the pore or channel depth is about 0.5 μm to about 1 μm, about 0.5 μm to about 5 μm, about 0.5 μm to about 10 μm, about 0.5 μm to about 15 μm, about 0.5 μm to about 20 μm, about 1 μm to about 5 μm, about 1 μm to about 10 μm, about 1 μm to about 15 μm, about 1 μm to about 20 μm, about 5 μm to about 10 μm, about 5 μm to about 15 μm, about 5 μm to about 20 μm, about 10 μm to about 15 μm, about 10 μm to about 20 μm, about 15 μm to about 20 μm. In some cases, the pore or channel depth is about 0.5 μm, about 1 μm, about 5 μm, about 10 μm, about 15 μm, about 20 μm. In some cases, the pore or channel depth is at least about 0.5 μm, at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 15 μm, at least about 20 μm. In some cases, the pore or channel depth is at most about 0.5 μm, at most about 1 μm, at most about 2 μm, at most about 3 μm, at most about 4 μm, at most about 5 μm, at most about 6 μm, at most about 7 μm, at most about 8 μm, at most about 9 μm, at most about 10 μm. at most about 15 μm, or at most about 20 μm.

In some cases, the electroporation chip 150 comprises a plurality of pores with an average pore density of about 0.001 pore/μm² to about 10pores/μm². In some cases, the electroporation chip 150 comprises a plurality of pores with an average pore density of at least about 0.001 pore/μm², at least about 0.005 pore/μm², at least about 0.01 pore/μm², at least about 0.02 pore/μm², at least about 0.03 pore/μm², at least about 0.04 pore/μm², at least about 0.05 pore/μm², at least about 0.06 pore/μm², at least about 0.07 pore/μm², at least about 0.08 pore/μm², at least about 0.09 pore/μm², at least about 0.1 pore/μm², at least about 0.2 pore/μm², at least about 0.3 pore/μm², at least about 0.4 pore/μm², at least about 0.5 pore/μm², at least about 0.6 pore/μm², at least about 0.7 pore/μm², at least about 0.8 pore/μm², at least about 0.9 pore/μm², at least about 1 pore/μm², at least about 2 pores/μm², at least about 3 pores/μm², at least about 4 pores/μm², at least about 5 pores/μm², at least about 6 pores/μm², at least about 7 pores/μm², at least about 8 pores/μm², at least about 9 pores/μm², or at least about 10 pores/μm². In some cases, the electroporation chip 150 comprises a plurality of pores with an average pore density of at most about 0.001 pore/μm², at most about 0.005 pore/μm², at most about 0.01 pore/μm², at most about 0.02 pore/μm², at most about 0.03 pore/μm², at most about 0.04 pore/μm², at most about 0.05 pore/μm², at most about 0.06 pore/μm², at most about 0.07 pore/μm², at most about 0.08 pore/μm², at most about 0.09 pore/μm², at most about 0.1 pore/μm², at most about 0.2 pore/μm², at most about 0.3 pore/μm², at most about 0.4 pore/μm², at most about 0.5 pore/μm², at most about 0.6 pore/μm², at most about 0.7 pore/μm², at most about 0.8 pore/μm², at most about 0.9 pore/μm², at most about 1 pore/μm², at most about 2 pores/μm², at most about 3 pores/μm², at most about 4 pores/μm², at most about 5 pores/μm², at most about 6 pores/μm², at most about 7 pores/μm², at most about 8 pores/μm², at most about 9 pores/μm², or at most about 10 pores/μm².

In some cases, a cell chamber wall 155 and the electroporation chip 150 form an integral component which is removable from the first tube 110 (as depicted in FIG. 2E). In some cases, the outer circumference of the cell chamber wall 155 is approximately equal to the inner diameter of the first tube 110. In some cases, the outer circumference of the cell chamber wall provides a friction fit when disposed with the first tube 110. In some cases, the outer circumference of the cell chamber wall 155 abuts the end of the first tube where the first tube begins to taper to form a conical end. In some cases, the inner circumference of the cell chamber wall 155 comprises a diameter approximately equal to the diameter of the electroporation chip 150. In some cases, the electroporation chip 150 and the cell chamber wall 155 form an integral component. In some cases, the electroporation chip and the cell chamber wall are provided as a permeable cell culture insert.

In some cases, the electroporation system described herein comprises a cell chamber 164. In some cases, the cell chamber receives the plurality of cells to be subjected to electroporation. In some cases, the plurality of cells is provided in a suspension. In some cases, the cell chamber 164 is formed by a side of a stabilizer 168 and a first side of the electroporation chip 150. In some cases, the stabilizer 168 comprises an outer diameter approximately equal to an inner diameter of the first tube 110, such that a friction fit is formed between the stabilizer 168 and the first tube 110. In some cases, the fit between the stabilizer 168 and the first tube 110 provides a liquid-tight seal. In some cases, the fit between the stabilizer 168 and the first tube 110 provides an air-tight seal.

In some cases, the electroporation system described herein comprises a transfection reagent chamber 166. In some cases, the distal portion of the first tube 110 comprises the transfection reagent chamber. In some cases, the transfection reagent chamber is on a second side of the electroporation chip. In some cases, the transfection reagent chamber is located between the second side of the electroporation chip and the closed end of the first tube 110.

In some cases, transfection reagents are disposed within the transfection reagent chamber 166. In some cases, the transfection reagents have small molecular weight. In some cases, the transfection reagents have large molecular weight. In some cases, the molecular weight of the transfection reagents is about 100 g/mol to about 1000 g/mol. In some cases, the molecular weight of the transfection reagents is about 1000 g/mol to about 2000 g/mol. In some cases, the molecular weight of the transfection reagents is about 2000 g/mol to about 3000 g/mol. In some cases, the molecular weight of the transfection reagents is about 3000 g/mol to about 4000 g/mol. In some cases, the molecular weight of the transfection reagents is about 4000 g/mol to about 5000 g/mol. In some cases, the molecular weight of the transfection reagents is about 5000 g/mol to about 7500 g/mol. In some cases, the molecular weight of the transfection reagents is about 7500 g/mol to about 10000 g/mol. In some cases, the transfection reagents comprise DNAs, RNAs, proteins, other charged biomolecules, and/or charged molecules. In some specific cases, the transfection reagents comprise plasmid DNAs. In some specific cases, the transfection reagents comprise siRNAs. In some specific cases, the transfection reagents comprise mRNA, encapsulated in nanolipid particles or not. In some specific cases, the transfection reagents comprise miRNAs. In some specific cases, the transfection reagents comprise shRNAs. In some specific cases, the transfection reagents comprise a small-molecule drug. In some specific cases, the transfection reagents comprise polypeptides. In some specific cases, the transfection reagents comprise antibodies. In some specific cases, the transfection reagents comprise a combination of the above-described reagents.

In some cases, the electroporation system described herein is configured to conduct electroporation on a plurality of cells disposed within the centrifuge tube. In some cases, an electroporation chip 150 is disposed within the centrifuge tube. In some cases, the electroporation chip is sandwiched between a cell chamber 164 and a transfection reagent chamber 166. In some cases, the electroporation chip 164 provides a physical barrier between the transfection reagent chamber 166 and the cell chamber 164.

In some cases, the electroporation system described herein comprises two electrodes 130 and 135 for providing an electric field for cell electroporation or transfection. In some cases, a circuit comprises the first electrode 130 and the second electrode 135 and a power source. In some cases, the power source provides an electric potential between the first electrode 130 and the second electrode 135 to produce an electric field across electroporation chip 150. In some cases, the first electrode 130 is a positive electrode. In some cases, the second electrode 135 is a negative electrode. In some cases, the first electrode 130 is a negative electrode. In some cases, the second electrode 135 is a positive electrode. In some cases, the first electrode 130 is an anode. In some cases, the second electrode 135 is a cathode. In some cases, the first electrode 130 is a cathode. In some cases, the second electrode 135 is an anode. In some cases, a first electrode contact 140 is provided to couple the first electrode 130 via a first electrode wire 142, to the power source. In some cases, a second electrode contact 145 is provided to couple the second electrode 135 to the power source.

In some cases, a first electrode 130 is provided on a first side of the electroporation chip. In some cases, a first electrode 130 is disposed within the cell chamber 164 of the centrifuge tube. In some cases, the first electrode 130 is electrically coupled to a first electrode contact 140 via an electrode wire 142. In some cases, the first electrode contact 140 is provided on an exterior of the first tube 110. In some cases, the first electrode contact 140 provides an electrical connection point for connecting to an electrical circuit external to the centrifuge tube. In some cases, the first electrode contact 140 provides an electric potential to the first electrode 130 when connected to an electric power source. In some cases, the first electrode wire 142 runs through the second tube 120 from the first electrode contact 140 to the first electrode 130. In some cases, an aperture is provided through the cap 115 to allow the electrode wire to pass through a wall of the cap. In some cases, an aperture is provided through the stabilizer to allow an electrical connection of the electrode wire 142 of a first side of the stabilizer to the first electrode 130 on a second side of the stabilizer. In some cases, the cap 115, the second tube 120, the first electrode contact 140, electrode wire 142, and first electrode 130 are coupled, such that coupling of the cap 115 to the first tube 110 positions the first electrode 130 within the cell chamber 164. In some cases, the first electrode contact is provided along a center axis of the centrifuge tube 105.

In some cases, a second electrode 135 is provided within the transfection reagent chamber 166. In some cases, a second electrode 135 is provided on a second side of the electroporation chip. In some cases, the second electrode 135 is coupled to the first tube 110 at the conical end. In some cases, the second electrode 135 is in electrical communication with a second electrode contact 145 In some cases, the second electrode contact 145 provides an electric potential to the second electrode 135 when connected to an electric power source. In some cases, the second electrode contact is provided along a center axis of the centrifuge tube 105. In some cases, second electrode contact 145 is provided at the exterior of the first tube 110. In some cases, second electrode contact 145 is proximal to the closed end of the first tube 110. In some cases, the second electrode contact 145 is provided on a side of the first tube 110 opposite side of the first electrode contact 140. In some cases, the connection between the second electrode 135 and the second electrode contact 145 runs through an outer wall of the end of the first tube 110.

The first electrode contact 140 and the second electrode contact 145 may be coupled to an electrical circuit, such that an electric potential provided between the first electrode 130 and the second electrode 135 and across the electroporation chip 150. In some cases, first electrode 130 and the second electrode 135 are configured to create an electric field across the electroporation chip 150. In some cases, application of an electric field across the electroporation chip increases permeability of cell membranes present within the cell chamber 164 for transfection by reagents provided within the transfection reagent chamber 166. In some cases, the first electrode 130 and second electrode 135 comprise platinum electrodes. In some cases, the first electrode 130 and second electrode 135 comprise copper, graphite, titanium, brass, silver, gold, or other suitable materials. In some cases, the first electrode 130 is configured as a cathode. In some cases, the second electrode 135 is configure as an anode.

In some cases, the electroporation system described herein further comprises a structure that extend the exterior of the centrifuge tube and the cell chamber. In some specific cases, the electroporation system described herein further comprises a syringe 160. In other specific cases, the electroporation system described herein further comprises a serological pipette.

In some cases, the electroporation system described herein further comprises a stabilizer 168 that serves to hold the syringe 160 described herein and the first electrode wire 142 or effectively the first electrode 140 in place during the centrifugation. In some cases, the stabilizer described herein comprises an electrode wire aperture such that the first electrode wire 142 passes through the electrode wire aperture to provide an electrical connection from the first electrode 130 to the first electrode contact 140. In some specific cases, the stabilizer holds the first electrode wire 142 in place. In some cases, the stabilizer described herein comprises a syringe through hole 162, for receiving the syringe 160 to provide cells to the cell chamber 164. In some cases, a friction fit is provided between the stabilizer 168 and the first tube 110. In some specific cases, the friction fit facilitates the retention of the second tube 120 within the first tube 110. In some cases, the stabilizer 168 provides a liquid tight seal of the cell chamber 164. In some cases, the stabilizer 168 provides an air-tight seal of the cell chamber.

In some cases, the electroporation system described herein further comprises a cap. In some cases, the cap described herein is removable. In some cases, the cap described herein comprises an aperture from where the first electrode wire 142 passes through. In some cases, the cap described herein comprises a syringe aperture for receiving a syringe 160 to provide a plurality of cells to the cell chamber 164. In some specific cases, the syringe aperture described herein is positioned off a center axis of the cap.

In some cases, the cells are injected into the cell chamber via a syringe 160. In some cases, the cap 115 comprises a syringe aperture 161 to allow a syringe 160 to pass through the cap and into the second tube 120. In some cases, the stabilizer 168 comprises a syringe through hole 162 to allow a syringe 160 to pass through the stabilizer and into the cell chamber 164. In some cases, the first syringe aperture and the second syringe aperture comprise one-way seals to prevent unwanted expulsion of cells in a suspension from the cell chamber 164. In some cases, the syringe is used to withdraw cells, which have undergone electroporation, from the cell chamber 164. In some cases, withdrawn cells are then incubated.

In some cases, the electroporation system described herein comprises a plurality of tube holders for simultaneous electroporation of one or more samples provided in a plurality of centrifuge tubes described herein. In some specific cases, the electroporation system described herein comprises at least two tube holders for simultaneous electroporation. In some specific cases, the electroporation system described herein comprises at least three tube holders for simultaneous electroporation. In some specific cases, the electroporation system described herein comprises at least four tube holders for simultaneous electroporation. In some specific cases, the electroporation system described herein comprises at least five tube holders for simultaneous electroporation. In some specific cases, the electroporation system described herein comprises at least six tube holders for simultaneous electroporation. In some specific cases, the electroporation system described herein comprises at least seven, eight, night, ten, eleven, twelve, thirteen, fourteen, fifteen, sixteen, seventeen, eighteen, nineteen, twenty, twenty-one, twenty-two, twenty-three, or twenty-four tube holders for simultaneous electroporation.

In some cases, the electroporation system described herein comprises a rotor 280. In some cases. The rotor 280 comprises a hub. In some cases, the rotor 280 described herein is rotated to produce a relative centrifugal force (RCF) of about 1 to 3000 g. In some cases, the centrifuge rotates at about 1 g to about 50 g, about 1 g to about 100 g, about 1 g to about 300 g, about 1 g to about 500 g, about 1 g to about 700 g, about 1 g to about 1,000 g, about 1 g to about 1,500 g, about 1 g to about 2,000 g, about 1 g to about 2,500 g, about 1 g to about 3,000 g, about 50 g to about 100 g, about 50 g to about 300 g, about 50 g to about 500 g, about 50 g to about 700 g, about 50 g to about 1,000 g, about 50 g to about 1,500 g, about 50 g to about 2,000 g, about 50 g to about 2,500 g, about 50 g to about 3,000 g, about 100 g to about 300 g, about 100 g to about 500 g, about 100 g to about 700 g, about 100 g to about 1,000 g, about 100 g to about 1,500 g, about 100 g to about 2,000 g, about 100 g to about 2,500 g, about 100 g to about 3,000 g, about 300 g to about 500 g, about 300 g to about 700 g, about 300 g to about 1,000 g, about 300 g to about 1,500 g, about 300 g to about 2,000 g, about 300 g to about 2,500 g, about 300 g to about 3,000 g, about 500 g to about 700 g, about 500 g to about 1,000 g, about 500 g to about 1,500 g, about 500 g to about 2,000 g, about 500 g to about 2,500 g, about 500 g to about 3,000 g, about 700 g to about 1,000 g, about 700 g to about 1,500 g, about 700 g to about 2,000 g, about 700 g to about 2,500 g, about 700 g to about 3,000 g, about 1,000 g to about 1,500 g, about 1,000 g to about 2,000 g, about 1,000 g to about 2,500 g, about 1,000 g to about 3,000 g, or about 1,500 g to about 2,000 g, about 1,500 g to about 2,500 g, about 1,500 g to about 3,000 g, about 2,000 g to about 2,500 g, about 2,000 g to about 3,000 g, or about 2,500 g to about 3,000 g. In some cases, the centrifuge rotates at about 1 g, about 50 g, about 100 g, about 300 g, about 500 g, about 700 g, about 1,000 g, about 1,500 g, or about 2,000 g. In some cases, the centrifuge rotates at least about 1 g, about 50 g, about 100 g, about 300 g, about 500 g, about 700 g, about 1,000 g, or about 1,500 g. In some cases, the centrifuge rotates at most about 50 g, about 100 g, about 300 g, about 500 g, about 700 g, about 1,000 g, about 1,500 g, about 2,000 g, about 2,500 g, about 3,000 g.

With reference to FIGS. 1B and 1C, an internal depiction of centrifuge tubes 205 loaded into a centrifuge of the electroporation system is depicted. In some cases, the centrifuge tubes 205 are loaded into tube holders 255. In some cases, the centrifuge tubes are configured for electroporation of cells, as disclosed herein. In some cases, the tube holders 255 are pivoting tube holders connected to a rotor 280 of the centrifuge via a hinge or pivotable coupling 260, such that the tube holders 255, and centrifuge tubes 205 provided in the tube holders, rotate under the influence of a centrifugal force provided by the centrifuge. In some cases, the tube holders 255 are pivotably coupled to the hub such that the tube holders 255 rotate towards about a horizontal orientation or towards an exactly horizontal orientation as the rotor is rotated. In some cases, the tube holders 255 are pivotably coupled to the hub such that the tube holders 255 rotate towards an angled orientation as the rotor is rotated. In some cases, the centrifuge tubes 205 is rotatable around the hub. In some cases, the centrifuge tubes 205 pivots under influence of a centrifugal force applied by the rotor 280.

In some cases, the electroporation system described herein comprises a swing-bucket rotor. In the cases where a swing-bucket rotor is used, the tube holders 255 are pivotably coupled to the hub such that the tube holders 255 rotate towards an exactly horizontal orientation or towards about a horizontal orientation as the rotor is rotated. In some cases, the electroporation system described herein comprises a fixed-angel rotor. In the cases where a fixed-angel rotor is used, the tube holders 255 are pivotably coupled to the hub such that the tube holders 255 rotate towards an angled orientation as the rotor is rotated.

In some cases, a circuit is provided through the centrifuge device of the electroporation system to provide an electrical connection from a power source 270 to electrodes of a centrifuge tube 205 configured for electroporation of cells, as disclosed herein. In some cases, the power source 270 is external to the centrifuge 300. In some cases, the power source 270 is within the centrifuge 300. In some cases, the electroporation centrifuge system is configured, such that the electrical connection from the power source 270 to electrodes of the centrifuge tubes 205 is only established when the centrifuge tubes are provided at the desired angle after pivoting under influence of the centrifugal force provided by the centrifuge. In some cases, a first electrode contact (140 as depicted in FIG. 1A) extending from the centrifuge tube 205 only contacts a first electrical contact 275 of a circuit when the centrifuge tube is provided at the desired angle due to the centrifugal forces acting on the tube holder and tube during the rotation of the centrifuge. In some specific cases, a first electrode contact 140 only contacts a first electrical contact 275 of a circuit when the centrifuge tube is about perpendicular (or exactly perpendicular) to the center axis of the centrifuge rotor during the rotation of the centrifuge. In some specific cases, a first electrode contact 140 always contacts a first electrical contact 275 of a circuit, and a connector with switch is installed between the power source and the circuit. In some cases, a second electrode contact (145 as depicted in FIG. 1A) is provided in electrical communication with a power source by a second electrical contact 277 of a circuit. In some cases, the second electrical contact 277 is provided by a chassis, including a rotation frame, of the centrifuge the electric connector on the outer chassis surface. In some cases, when the tube is placed into the centrifuge tube holder, the second electrical contact (145 as depicted in FIG. 1A) contacts the rotation frame. In some cases, conductive strip is placed from at least one centrifuge tube, or each centrifuge tube, location to a central screw location. In some cases, a holder with conductive strips on the outer surface and an electrical socket on the top of the holder is tightly mounted onto a central screw. In some cases, lead wires 271, 273 provide an electrical communication of the electrical contacts 275, 277 of the circuit.

In some cases, the electroporation system comprises a rotatable electrical coupling 279 to maintain an electrical communication between a power source 270 and the circuit within the centrifuge device of the electroporation system during the rotation of the centrifugation. In some specific cases, the rotatable electrical coupling 279 comprises a slip ring connector, which rotates together with the rotor during the rotation of the centrifugation.

With reference to FIG. 1D, a representation of a plurality of cells 220 within a suspension provided in a centrifuge tube are depicted under a centrifugal force 299, according to some cases. In some cases, under a centrifugal force 299, the plurality of cells 220 are pushed toward and against a surface the electroporation chip 250 within the centrifuge tube to provide efficient electroporation of the cells.

In some cases, the tube holders 255 pivot to an angle about perpendicular (or exactly perpendicular) to the center axis 290 of the centrifuge rotor 280. In some cases, a stop wall, or barrier 265 is provided to stop rotation of a tube holder at the desired angle. In some cases, the angle about perpendicular to the center axis 290 corresponds to an angle which is about perpendicular (or exactly perpendicular) to the force of acceleration due to gravity.

In some cases, a connector provides electrical communication between the power source and the circuit described herein. With reference to FIGS. 1F and 1G, a centrifuge 300 configured for providing an electrical current to one or more centrifuge tubes 350 is depicted, according to some cases. In some cases, the centrifuge 300 comprises a centrifuge cover 310. In some cases, the centrifuge 300 comprises a rotatable electrical coupling 379 to mate with a power source connector 378 when the cover 310 is closed. In some cases, an external power source connects to a plug on the outer surface of the cover. In some cases, the plug comprises a first outer terminal 381 and a second outer terminal 383. In some cases, the first outer terminal 381 is a positive terminal, and the second outer terminal 383 is a negative terminal. In some cases, the first terminal 381 is in electrical communication with a first inner terminal 371 of the power source connector 378 provided on the inside surface of the cover 310. In some cases, the second terminal 383 is in electrical communication with a second inner terminal 373 of the power source connector 378 provided on the inside surface of the cover 310.

In some cases, when the lid is closed, the rotatable electrical coupling 379 mates with a power source connector such that the input (e.g. negative input signal) from the power source connected to the second outer terminal 383 to transferred through the second inner terminal 373 and to one or more second electrical contacts (e.g. second electrical contact 277 depicted in FIGS. 1F and 1G) provided on tube holders 355. In some cases, the second electrical contacts provided on tube holders 355 make contact with second electrode contacts (e.g. second electrode contact 145 as depicted FIG. 1) of centrifuge tubes 305 configured for electroporation when the tubes are placed within the tube holders.

In some cases, when the lid is closed, the rotatable electrical coupling 379 mates with a power source connector such that the input (e.g. positive input signal) from the power source connected to the first outer terminal 381 to transferred through the second inner terminal 371 and to conductive strips 375 provided on a rotor 350 of the centrifuge 300. In some cases, first electrode contacts 340 of a centrifuge tubes 305 placed in the tube holders 355 contact the conductive strips 375 as the tube holders pivot under the influence of centrifugal forces created by the rotation of the centrifuge. In some cases, contact of a first electrode contact 340 to a conductive strip 375 completes the circuit, provided an electric potential between the first and second electrodes (e.g. 130 and 135 depicted in FIG. 1) of the centrifuge tube 350 and provides an electric field across an electroporation chip within the tube to electroporate cells. In some cases, the first electrode contacts 340 further comprise a spring to facilitate contact with the conductive strips. In some cases, conductive silver paste is placed in the socket and the bottom of the centrifuge tube to improve electrical connection with low resistance.

In some cases, a current is applied to a circuit comprising the first electrode 130 and the second electrode 135 to produce an electric field. In some cases, the current applied to the first electrode 130 and the second electrode 135 at a voltage/distance between the two electrodes as 0.5 V/cm to 1000V/cm, including increments therein. In some cases, the voltage is applied at as a pulse. In some cases, the pulse length is 1 to 50 milliseconds (ms) including increments therein. In some cases, the voltage is applied as a series of pulses. In some cases, the series of pulses comprises 1 to 100 pulses. In some cases, the pulses are applied as a square wave signal. In some cases, the duration between pulses (no voltage applied, pulse interval) is approximately equal to the selected pulse duration. For example, if a pulse length of 10 ms is utilized, the pulse cycle includes a voltage application for 10 ms followed by a period of no voltage application for 10 ms. In other cases, the pulse interval is longer than the selected pulse duration. For example, if a pulse length of 10 ms is utilized, the pulse cycle includes a voltage application for 10 ms followed by a period of no voltage application for 100 ms. In other cases, the pulse interval is shorter than the selected pulse duration.

In some cases, the applied voltage/distance between the two electrodes is about 0.5 V/cm to about 1,000 V/cm. In some cases, the applied voltage/distance between the two electrodes is about 0.5 V/cm to about 1 V/cm, about 0.5 V/cm to about 100 V/cm, about 0.5 V/cm to about 300 V/cm, about 0.5 V/cm to about 500 V/cm, about 0.5 V/cm to about 800 V/cm, about 0.5 V/cm to about 1,000 V/cm, about 1 V/cm to about 100 V/cm, about 1 V/cm to about 300 V/cm, about 1 V/cm to about 500 V/cm, about 1 V/cm to about 800 V/cm, about 1 V/cm to about 1,000 V/cm, about 100 V/cm to about 300 V/cm, about 100 V/cm to about 500 V/cm, about 100 V/cm to about 800 V/cm, about 100 V/cm to about 1,000 V/cm, about 300 V/cm to about 500 V/cm, about 300 V/cm to about 800 V/cm, about 300 V/cm to about 1,000 V/cm, about 500 V/cm to about 800 V/cm, about 500 V/cm to about 1,000 V/cm, or about 800 V/cm to about 1,000 V/cm. In some cases, the applied voltage/distance between the two electrodes is about 0.5 V/cm, about 1 V/cm, about 100 V/cm, about 300 V/cm, about 500 V/cm, about 800 V/cm, or about 1,000 V/cm. In some cases, the applied voltage is at least about 0.5 V/cm, about 1 V/cm, about 100 V/cm, about 300 V/cm, about 500 V/cm, or about 800 V/cm. In some cases, the applied voltage/distance between the two electrodes is at most about 1 V/cm, about 100 V/cm, about 300 V/cm, about 500 V/cm, about 800 V/cm, or about 1,000 V/cm.

In some cases, a pulse length is about 1 ms to about 50 ms. In some cases, a pulse duration is about 1 ms to about 5 ms, about 1 ms to about 10 ms, about 1 ms to about 15 ms, about 1 ms to about 20 ms, about 1 ms to about 30 ms, about 1 ms to about 50 ms, about 5 ms to about 10 ms, about 5 ms to about 15 ms, about 5 ms to about 20 ms, about 5 ms to about 30 ms, about 5 ms to about 50 ms, about 10 ms to about 15 ms, about 10 ms to about 20 ms, about 10 ms to about 30 ms, about 10 ms to about 50 ms, about 15 ms to about 20 ms, about 15 ms to about 30 ms, about 15 ms to about 50 ms, about 20 ms to about 30 ms, about 20 ms to about 50 ms, or about 30 ms to about 50 ms. In some cases, a pulse duration is about 1 ms, about 5 ms, about 10 ms, about 15 ms, about 20 ms, about 30 ms, or about 50 ms. In some cases, a pulse duration is at least about 1 ms, about 5 ms, about 10 ms, about 15 ms, about 20 ms, or about 30 ms. In some cases, a pulse duration is at most about 5 ms, about 10 ms, about 15 ms, about 20 ms, about 30 ms, or about 50 ms.

In some cases, a voltage cycle or series comprises about 1 pulse to about 200 pulses. In some cases, a voltage cycle comprises about 1 pulse to about 10 pulses, about 1 pulse to about 30 pulses, about 1 pulse to about 50 pulses, about 1 pulse to about 70 pulses, about 1 pulse to about 100 pulses, about 1 pulse to about 200 pulses, about 10 pulses to about 30 pulses, about 10 pulses to about 50 pulses, about 10 pulses to about 70 pulses, about 10 pulses to about 100 pulses, about 10 pulses to about 200 pulses, about 30 pulses to about 50 pulses, about 30 pulses to about 70 pulses, about 30 pulses to about 100 pulses, about 30 pulses to about 200 pulses, about 50 pulses to about 70 pulses, about 50 pulses to about 100 pulses, about 50 pulses to about 200 pulses, about 70 pulses to about 100 pulses, about 70 pulses to about 200 pulses, or about 100 pulses to about 200 pulses. In some cases, a voltage cycle comprises about 1 pulse, about 10 pulses, about 30 pulses, about 50 pulses, about 70 pulses, about 100 pulses, or about 200 pulses. In some cases, a voltage cycle comprises at least about 1 pulse, about 10 pulses, about 30 pulses, about 50 pulses, about 70 pulses, or about 100 pulses. In some cases, a voltage cycle comprises at most about 10 pulses, about 30 pulses, about 50 pulses, about 70 pulses, about 100 pulses, or about 200 pulses.

In some cases, the pulse interval is about 1 ms to about 200 ms. In some cases, the pulse interval is about 1 ms to about 5 ms, about 1 ms to about 10 ms, about 1 ms to about 20 ms, about 1 ms to about 30 ms, about 1 ms to about 40 ms, about 1 ms to about 50 ms, about 1 ms to about 100 ms, about 1 ms to about 200 ms, about 5 ms to about 10 ms, about 5 ms to about 20 ms, about 5 ms to about 30 ms, about 5 ms to about 40 ms, about 5 ms to about 50 ms, about 5 ms to about 100 ms, about 5 ms to about 200 ms, about 10 ms to about 20 ms, about 10 ms to about 30 ms, about 10 ms to about 40 ms, about 10 ms to about 50 ms, about 10 ms to about 100 ms, about 10 ms to about 200 ms, about 20 ms to about 30 ms, about 20 ms to about 40 ms, about 20 ms to about 50 ms, about 20 ms to about 100 ms, about 20 ms to about 200 ms, about 30 ms to about 40 ms, about 30 ms to about 50 ms, about 30 ms to about 100 ms, about 30 ms to about 200 ms, about 40 ms to about 50 ms, about 40 ms to about 100 ms, about 40 ms to about 200 ms, about 50 ms to about 100 ms, about 50 ms to about 200 ms, or about 100 ms to about 200 ms. In some cases, the pulse interval is about 1 ms, about 5 ms, about 10 ms, about 20 ms, about 30 ms, about 40 ms, about 50 ms, about 100 ms, or about 200 ms. In some cases, the pulse interval is at least about 1 ms, about 5 ms, about 10 ms, about 20 ms, about 30 ms, about 40 ms, about 50 ms, or about 100 ms. In some cases, the pulse interval is at most about 5 ms, about 10 ms, about 20 ms, about 30 ms, about 40 ms, about 50 ms, about 100 ms, or about 200 ms.

In some cases, a series or cycle of voltage pulses are applied as a waveform. In some cases, a series or cycle of voltage pulses are applied as a square waveform, a sinusoidal waveform, a triangular waveform, a sawtooth waveform, or a combination thereof.

In some cases, the centrifuge 300 of the electroporation system further comprises an operation panel 360. Operation panel 360 may allow a user to monitor the rate of rotation, the relative centrifugal force, and the duration of centrifuging. Operation panel 360 may allow a user to set the rate of rotation, the relative centrifugal force, and the duration of centrifuging.

In some cases, centrifuge 300 of the electroporation system comprises a start button 361 to start a centrifuge cycle. In some cases, centrifuge 300 comprises a stop button 362 to manually stop a centrifuge cycle. In some cases, the centrifuge comprises a latch 365. The latch may lock the cover in a closed position upon starting the centrifuge.

Further described herein is a method of using the electroporation system described herein comprising (in any combination or order) at least one of the following steps: providing a suspension comprising a plurality of cells to a surface of an electroporation chip; applying a centrifugal force to the plurality of cells, such that at least a portion of the plurality of cells is pressed against the surface of the electroporation chip; and providing an electrical voltage across the electroporation chip as the centrifugal force is applied to the plurality of cells. In some cases, the electric voltage is applied after the centrifugal force is applied to the plurality of cells.

Further described herein is a method of using the electroporation system described herein comprising providing a suspension comprising a plurality of cells to the first side of the electroporation chip; applying a centrifugal force to the plurality of cells, such that at least a portion of the plurality of cells is pressed against the first surface of the electroporation chip; and providing an electrical voltage across the electroporation chip as the centrifugal force is applied to the plurality of cells.

In some cases, the method of using the electroporation system described herein comprises providing a suspension comprising a plurality of cells to an enclosed space that is sterile. Often, this is a step that occurs early or initially in the method. In some cases, the method of using the electroporation system described herein comprises providing a suspension comprising a plurality of cells to a first surface of an electroporation chip as described herein (e.g., in Section I), or a different surface. In some cases, the suspension comprising a plurality of cells is provided within the cell chamber as described in herein (e.g., in Section I). In some specific cases, as depicted in FIG. 1A, the suspension comprising a plurality of cells is provided within the cell chamber 164 that is formed by a side of a stabilizer 168 and a first side of the electroporation chip 150.

In some cases, the suspension comprising a plurality of cells is provided through a sterile structure that is connected between the exterior of the centrifuge tube and the cell chamber described herein (e.g., in Section I). In some specific cases, the suspension comprising a plurality of cells is provided through a syringe 160 as depicted in FIG. 1A. In some specific cases, the suspension comprising a plurality of cells is provided through a serological pipette.

In some cases, the suspension comprising a plurality of cells comprises a saline-based buffer that is suitable for electroporation. In some cases, the suspension comprising a plurality of cells comprises a phosphate-based buffer that is suitable for electroporation. In some cases, the suspension comprising a plurality of cells comprises a HEPES-based buffer that is suitable for electroporation. In some cases, the suspension comprising a plurality of cells comprises a cell-culture-media-based buffer that is suitable for electroporation.

In some cases, the plurality of cells within the suspension are of a certain cell type; in some cases, the plurality of cells are a mixture of different cell types (e.g., PBMCs). In particular embodiments, the suspension comprises suspension cells, e.g., cells that do not typically adhere to a plate during cell culture. In some cases, the suspension comprises adherent cells. In some cases, the suspension comprises a plurality of eukaryotic cells. In some specific cases, the suspension comprises a plurality of mammalian cells. In some particular cases, the suspension comprises a plurality of human cells. In some cases, the suspension comprises non-human cells (e.g., rodents cells, primate cells, etc.). In some cases, the suspension comprises a plurality of primary cells, e.g., primary cells obtained from dissecting a target tissue or organ, or primary cells obtained from blood (e.g., PBMCs, PBLs, monocytes, macrophages, dendritic cells, lymphocytes, myeloid cells, stem cells, hematopoietic stem cells).

In some cases, the suspension comprises a plurality of cells from a cell line. In some cases, the plurality of cells from a cell line is from a suspension cell line or an adherent cell line. In some specific cases, the cell line described herein is of a myeloma origin. In some specific cases, the cell line described herein is of a lymphoma origin. In some specific cases, the cell line described herein is of a leukemia origin. In some cases, the cell line described herein has an epithelial morphology. In some cases, the cell line described herein has an endothelial morphology. In some cases, the cell line described herein has a neuronal morphology. In some cases, the cell line described herein has an endothelial morphology. In some specific cases, the plurality of eukaryotic cells comprise human lung. In some specific cases, the plurality of eukaryotic cells comprise human cervix. In some specific cases, the plurality of eukaryotic cells comprise African green monkey kidney. In some specific cases, the plurality of eukaryotic cells comprise mouse embryo. In some specific cases, the plurality of eukaryotic cells comprise mouse connective tissue. In some specific cases, the plurality of eukaryotic cells comprise Chinese hamster ovary. In some specific cases, the plurality of eukaryotic cells comprise Syrian hamster kidney. In some specific cases, the plurality of eukaryotic cells are derived from human kidney. In some specific cases, the plurality of eukaryotic cells are derived from human liver. In some specific cases, the plurality of eukaryotic cells are derived from bovine aorta. In some specific cases, the plurality of eukaryotic cells are derived from human neuroblastoma. In some specific cases, the plurality of eukaryotic cells are derived from mouse myeoloa. In some specific cases, the plurality of eukaryotic cells are derived from human hystiocytic lymphoma. In some specific cases, the plurality of eukaryotic cells are derived from human leukemia. In some specific cases, the plurality of eukaryotic cells are derived from mouse B-cell lymphoma. In some specific cases, the plurality of eukaryotic cells are derived from mouse lymphoma. In some specific cases, the plurality of eukaryotic cells are derived from human myeloma. In some specific cases, the plurality of eukaryotic cells are derived from human T-cell leukemia. In some specific cases, the plurality of eukaryotic cells are derived from human monocyte leukemia. In some specific cases, the plurality of eukaryotic cells comprise mouse embryonic fibroblasts (MEF). In some specific cases, the plurality of eukaryotic cells comprise human embryonic fibroblasts (HEF). In some specific cases, the plurality of eukaryotic cells comprise dendritic cells. In some specific cases, the plurality of eukaryotic cells comprise mesenchymal stem cells. In some specific cases, the plurality of eukaryotic cells comprise bone marrow-derived dendritic cells. In some specific cases, the plurality of eukaryotic cells comprise bone marrow derived stromal cells. In some specific cases, the plurality of eukaryotic cells comprise adipose stromal cells. In some specific cases, the plurality of eukaryotic cells comprise enucleated cells. In some specific cases, the plurality of eukaryotic cells comprise neural stem cells. In some specific cases, the plurality of eukaryotic cells comprise immature dendritic cells. In some specific cases, the plurality of eukaryotic cells comprise immune cells. In some specific cases, the plurality of eukaryotic cells comprise NS0. In some specific cases, the plurality of eukaryotic cells comprise U937. In some specific cases, the plurality of eukaryotic cells comprise HL60. In some specific cases, the plurality of eukaryotic cells comprise WEHI231. In some specific cases, the plurality of eukaryotic cells comprise YAC1. In some specific cases, the plurality of eukaryotic cells comprise U266B1. In some specific cases, the plurality of eukaryotic cells comprise Jurkat. In some specific cases, the plurality of eukaryotic cells comprise THP-1. In some specific cases, the plurality of eukaryotic cells comprise MRC-5. In some specific cases, the plurality of eukaryotic cells comprise HeLa. In some specific cases, the plurality of eukaryotic cells comprise HEK-293 or HEK-293T cells. In some specific cases, the plurality of eukaryotic cells comprise HepG2. In some specific cases, the plurality of eukaryotic cells comprise SH-SY5Y. In some specific cases, the plurality of eukaryotic cells comprise MCF-7. In some specific cases, the plurality of eukaryotic cells comprise Sf9, Vero, NIH3T3, L929, CHO, BHK-21, Cos7, or BAE-1.

In some cases, the suspension described herein comprises a plurality of cells with a low elastic modulus (effective Young's modulus). In some cases, the suspension described herein comprises a plurality of cells with a Poisson ratio of about 0.5. In some cases, the suspension described herein comprises a plurality of cells with a Poisson ratio of about 0.4. In some cases, the suspension described herein comprises a plurality of cells with a Poisson ratio of about 0.3.

In some cases, the suspension comprising a plurality of cells at about 1×10² to about 1×10³ cells/mL. In other cases, the suspension comprising a plurality of cells at about 1×10³ to about 1×10⁴ cells/mL. In other cases, the suspension comprising a plurality of cells at about 1×10⁴ to about 1×10⁵ cells/mL. In other cases, the suspension comprising a plurality of cells at about 1×10⁵ to about 1×10⁶ cells/mL. In other cases, the suspension comprising a plurality of cells at about 1×10⁶ to about 1×10⁷ cells/mL.

In some cases, the second step comprises applying centrifugal force to the plurality of cells, such that at least a portion of the plurality of cells is pressed against the first surface of the electroporation chip.

In some cases, while an electric field is being applied across an electroporation chip of a centrifuge tube, as described herein, the centrifuge rotates to produce a relative centrifugal force (RCF) of about 1 to 3000 g. In some cases, the centrifuge rotates at about 1 g to about 50 g, about 1 g to about 100 g, about 1 g to about 300 g, about 1 g to about 500 g, about 1 g to about 700 g, about 1 g to about 1,000 g, about 1 g to about 1,500 g, about 1 g to about 2,000 g, about 1 g to about 2,500 g, about 1 g to about 3,000 g, about 50 g to about 100 g, about 50 g to about 300 g, about 50 g to about 500 g, about 50 g to about 700 g, about 50 g to about 1,000 g, about 50 g to about 1,500 g, about 50 g to about 2,000 g, about 50 g to about 2,500 g, about 50 g to about 3,000 g, about 100 g to about 300 g, about 100 g to about 500 g, about 100 g to about 700 g, about 100 g to about 1,000 g, about 100 g to about 1,500 g, about 100 g to about 2,000 g, about 100 g to about 2,500 g, about 100 g to about 3,000 g, about 300 g to about 500 g, about 300 g to about 700 g, about 300 g to about 1,000 g, about 300 g to about 1,500 g, about 300 g to about 2,000 g, about 300 g to about 2,500 g, about 300 g to about 3,000 g, about 500 g to about 700 g, about 500 g to about 1,000 g, about 500 g to about 1,500 g, about 500 g to about 2,000 g, about 500 g to about 2,500 g, about 500 g to about 3,000 g, about 700 g to about 1,000 g, about 700 g to about 1,500 g, about 700 g to about 2,000 g, about 700 g to about 2,500 g, about 700 g to about 3,000 g, about 1,000 g to about 1,500 g, about 1,000 g to about 2,000 g, about 1,000 g to about 2,500 g, about 1,000 g to about 3,000 g, or about 1,500 g to about 2,000 g, about 1,500 g to about 2,500 g, about 1,500 g to about 3,000 g, about 2,000 g to about 2,500 g, about 2,000 g to about 3,000 g, or about 2,500 g to about 3,000 g. In some cases, the centrifuge rotates at about 1 g, about 50 g, about 100 g, about 300 g, about 500 g, about 700 g, about 1,000 g, about 1,500 g, or about 2,000 g. In some cases, the centrifuge rotates at least about 1 g, about 50 g, about 100 g, about 300 g, about 500 g, about 700 g, about 1,000 g, or about 1,500 g. In some cases, the centrifuge rotates at most about 50 g, about 100 g, about 300 g, about 500 g, about 700 g, about 1,000 g, about 1,500 g, about 2,000 g, about 2,500 g, about 3,000 g.

In some cases, while an electric field is being applied across an electroporation chip of a centrifuge tube, as described herein, the centrifuge rotates for a duration of 1 minute to an hour to complete an electroporation cycle. In some cases, the centrifuge rotates for about 1 minute to about 120 minutes. In some cases, the centrifuge rotates for about 1 minute to about 3 minutes, about 1 minute to about 5 minutes, about 1 minute to about 7 minutes, about 1 minute to about 10 minutes, about 1 minute to about 15 minutes, about 1 minute to about 30 minutes, about 1 minute to about 45 minutes, about 1 minute to about 60 minutes, about 1 minute to about 120 minutes, about 3 minutes to about 5 minutes, about 3 minutes to about 7 minutes, about 3 minutes to about 10 minutes, about 3 minutes to about 15 minutes, about 3 minutes to about 30 minutes, about 3 minutes to about 45 minutes, about 3 minutes to about 60 minutes, about 3 minutes to about 120 minutes, about 5 minutes to about 7 minutes, about 5 minutes to about 10 minutes, about 5 minutes to about 15 minutes, about 5 minutes to about 30 minutes, about 5 minutes to about 45 minutes, about 5 minutes to about 60 minutes, about 5 minutes to about 120 minutes, about 7 minutes to about 10 minutes, about 7 minutes to about 15 minutes, about 7 minutes to about 30 minutes, about 7 minutes to about 45 minutes, about 7 minutes to about 60 minutes, about 7 minutes to about 120 minutes, about 10 minutes to about 15 minutes, about 10 minutes to about 30 minutes, about 10 minutes to about 45 minutes, about 10 minutes to about 60 minutes, about 10 minutes to about 120 minutes, about 15 minutes to about 30 minutes, about 15 minutes to about 45 minutes, about 15 minutes to about 60 minutes, about 15 minutes to about 120 minutes, about 30 minutes to about 45 minutes, about 30 minutes to about 60 minutes, about 30 minutes to about 120 minutes, about 45 minutes to about 60 minutes, about 45 minutes to about 120 minutes, or about 60 minutes to about 120 minutes. In some cases, the centrifuge rotates for about 1 minute, about 3 minutes, about 5 minutes, about 7 minutes, about 10 minutes, about 15 minutes, about 30 minutes, about 45 minutes, about 60 minutes, or about 120 minutes. In some cases, the centrifuge rotates for at least about 1 minute, about 3 minutes, about 5 minutes, about 7 minutes, about 10 minutes, about 15 minutes, about 30 minutes, about 45 minutes, or about 60 minutes. In some cases, the centrifuge rotates for at most about 3 minutes, about 5 minutes, about 7 minutes, about 10 minutes, about 15 minutes, about 30 minutes, about 45 minutes, about 60 minutes, or about 120 minutes.

In some cases, the centrifugal force improves contact between the cells and the nanopores, particularly the apertures of the nanopores present on the surface of the chip. In some cases, elongation or flattening of the cells improves contact between the cells and the nanopores.

In some cases, at least 10% of the cells are pressed against the first surface of the electroporation chip. In other cases, at least 20% of the cells are pressed against the first surface of the electroporation chip. In other cases, at least 30% of the cells are pressed against the first surface of the electroporation chip. In other cases, at least 40% of the cells are pressed against the first surface of the electroporation chip. In other cases, at least 50% of the cells are pressed against the first surface of the electroporation chip. In other cases, at least 60% of the cells are pressed against the first surface of the electroporation chip. In other cases, at least 70% of the cells are pressed against the first surface of the electroporation chip. In other cases, at least 80% of the cells are pressed against the first surface of the electroporation chip. In other cases, at least 90% of the cells are pressed against the first surface of the electroporation chip. In other cases, at least 100% of the cells are pressed against the first surface of the electroporation chip.

In some cases, the cells that are pressed against the first surface of the electroporation chip are elongated to have a longer axis of about 110% of their original diameter. In other cases, the cells that are pressed against the first surface of the electroporation chip are elongated to have a longer axis of about 120% of their original diameter. In other cases, the cells that are pressed against the first surface of the electroporation chip are elongated to have a longer axis of about 130% of their original diameter. In other cases, the cells that are pressed against the first surface of the electroporation chip are elongated to have a longer axis of about 140% of their original diameter. In other cases, the cells that are pressed against the first surface of the electroporation chip are elongated to have a longer axis of about 150% of their original diameter. In other cases, the cells that are pressed against the first surface of the electroporation chip are elongated to have a longer axis of about 160%, about 170%, about 180%, about 190%, about 200%, about 250%, about 300%, about 400%, about 500% of their original diameter. In some cases, the cells that are pressed against the first surface of the electroporation chip are elongated to have a longer axis of at least about 160%, about 170%, about 180%, about 190%, about 200%, about 250%, about 300%, about 400%, about 500% of their original diameter. In some cases, the cells that are pressed against the first surface of the electroporation chip are elongated to have a longer axis of at most about 160%, about 170%, about 180%, about 190%, about 200%, about 250%, about 300%, about 400%, about 500% of their original diameter.

In some cases, the cells that are pressed against the first surface of the electroporation chip are flattened to have a shorter axis of about 90% of their original diameter. In other cases, the cells that are pressed against the first surface of the electroporation chip are flattened to have a shorter axis of about 80% of their original diameter. In other cases, the cells that are pressed against the first surface of the electroporation chip are flattened to have a shorter axis of about 70% of their original diameter. In other cases, the cells that are pressed against the first surface of the electroporation chip are flattened to have a shorter axis of about 60% of their original diameter. In other cases, the cells that are pressed against the first surface of the electroporation chip are flattened to have a shorter axis of about 50% of their original diameter. In other cases, the cells that are pressed against the first surface of the electroporation chip are flattened to have a shorter axis of about 40%, about 30%, about 20%, or about 10% of their original diameter. In some cases, the cells that are pressed against the first surface of the electroporation chip are flattened to have a shorter axis of at least about 90%, about 80%, about 70%, about 60%, about 50%, about 40%, about 30%, about 20%, or about 10% of their original diameter. In some cases, the cells that are pressed against the first surface of the electroporation chip are flattened to have a shorter axis of at most about 90%, about 80%, about 70%, about 60%, about 50%, about 40%, about 30%, about 20%, or about 10% of their original diameter.

In some cases, the cells that are pressed have an increased contact area with the first surface of the electroporation chip by about 10%. In other cases, the cells that are pressed have an increased contact area with the first surface of the electroporation chip by about 20%. In other cases, the cells that are pressed have an increased contact area with the first surface of the electroporation chip by about 30%. In other cases, the cells that are pressed have an increased contact area with the first surface of the electroporation chip by about 40%. In other cases, the cells that are pressed have an increased contact area with the first surface of the electroporation chip by about 50%. In other cases, the cells that are pressed have an increased contact area with the first surface of the electroporation chip by about 60%, about 70%, about 80%, about 90%, about 100%, about 150%, about 200%, about 250%, or about 300%. In some cases, the cells that are pressed have an increased contact area with the first surface of the electroporation chip by at least about 60%, about 70%, about 80%, about 90%, about 100%, about 150%, about 200%, about 250%, or about 300%. In some cases, the cells that are pressed have an increased contact area with the first surface of the electroporation chip by at most about 60%, about 70%, about 80%, about 90%, about 100%, about 150%, about 200%, about 250%, or about 300%.

In some cases, the gap between the cells that are pressed against the first surface of the electroporation chip and the nearest pores of the electroporation chip is reduced by about 10%, about 20%, about 30%, about 40%, about 50%, about 60%, about 70%, about 80%, or about 90%.

In some cases, the method of using the electroporation system described herein comprises providing an electrical voltage across the electroporation chip as the centrifugal force is applied to the plurality of cells. In specific cases, the electrical voltage is provided during the centrifugal force is applied. In specific cases, the electrical voltage is provided after the centrifugal force is applied. In specific cases, the centrifugal force is applied first before the electrical voltage is provided, then the centrifugal force is applied during the electrical voltage is provided. The current described herein is applied to a circuit comprising the first electrode 130 and the second electrode 135 to produce an electric field. In some cases, the current applied to the first electrode 130 and the second electrode 135 at a voltage/distance between two electrodes as 0.5 V/cm to 1000V/cm, including increments therein. In some cases, the voltage is applied at as a pulse. In some cases, the pulse length is 1 to 50 milliseconds (ms) including increments therein. In some cases, the voltage is applied as a series of pulses. In some cases, the series of pulses comprises 1 to 100 pulses. In some cases, the pulses are applied as a square wave signal. In some cases, the duration between pulses (no voltage applied, pulse interval) is approximately equal to the selected pulse duration. For example, if a pulse length of 10 ms is utilized, the pulse cycle includes a voltage application for 10 ms followed by a period of no voltage application for 10 ms. In other cases, the pulse interval is longer than the selected pulse duration. For example, if a pulse length of 10 ms is utilized, the pulse cycle includes a voltage application for 10 ms followed by a period of no voltage application for 100 ms. In other cases, the pulse interval is shorter than the selected pulse duration.

In some cases, the applied voltage/distance between two electrodes is about 0.5 V/cm to about 1,000 V/cm. In some cases, the applied voltage/distance between the two electrodes is about 0.5 V/cm to about 1 V/cm, about 0.5 V/cm to about 100 V/cm, about 0.5 V/cm to about 300 V/cm, about 0.5 V/cm to about 500 V/cm, about 0.5 V/cm to about 800 V/cm, about 0.5 V/cm to about 1,000 V/cm, about 1 V/cm to about 100 V/cm, about 1 V/cm to about 300 V/cm, about 1 V/cm to about 500 V/cm, about 1 V/cm to about 800 V/cm, about 1 V/cm to about 1,000 V/cm, about 100 V/cm to about 300 V/cm, about 100 V/cm to about 500 V/cm, about 100 V/cm to about 800 V/cm, about 100 V/cm to about 1,000 V/cm, about 300 V/cm to about 500 V/cm, about 300 V/cm to about 800 V/cm, about 300 V/cm to about 1,000 V/cm, about 500 V/cm to about 800 V/cm, about 500 V/cm to about 1,000 V/cm, or about 800 V/cm to about 1,000 V/cm. In some cases, the applied voltage/distance between the two electrodes is about 0.5 V/cm, about 1 V/cm, about 100 V/cm, about 300 V/cm, about 500 V/cm, about 800 V/cm, or about 1,000 V/cm. In some cases, the applied voltage is at least about 0.5 V/cm, about 1 V/cm, about 100 V/cm, about 300 V/cm, about 500 V/cm, or about 800 V/cm. In some cases, the applied voltage/distance between the two electrodes is at most about 1 V/cm, about 100 V/cm, about 300 V/cm, about 500 V/cm, about 800 V/cm, or about 1,000 V/cm.

In some cases, a pulse length is about 1 ms to about 50 ms. In some cases, a pulse duration is about 1 ms to about 5 ms, about 1 ms to about 10 ms, about 1 ms to about 15 ms, about 1 ms to about 20 ms, about 1 ms to about 30 ms, about 1 ms to about 50 ms, about 5 ms to about 10 ms, about 5 ms to about 15 ms, about 5 ms to about 20 ms, about 5 ms to about 30 ms, about 5 ms to about 50 ms, about 10 ms to about 15 ms, about 10 ms to about 20 ms, about 10 ms to about 30 ms, about 10 ms to about 50 ms, about 15 ms to about 20 ms, about 15 ms to about 30 ms, about 15 ms to about 50 ms, about 20 ms to about 30 ms, about 20 ms to about 50 ms, or about 30 ms to about 50 ms. In some cases, a pulse duration is about 1 ms, about 5 ms, about 10 ms, about 15 ms, about 20 ms, about 30 ms, or about 50 ms. In some cases, a pulse duration is at least about 1 ms, about 5 ms, about 10 ms, about 15 ms, about 20 ms, or about 30 ms. In some cases, a pulse duration is at most about 5 ms, about 10 ms, about 15 ms, about 20 ms, about 30 ms, or about 50 ms.

In some cases, the pulse interval is about 1 ms to about 200 ms. In some cases, the pulse interval is about 1 ms to about 5 ms, about 1 ms to about 10 ms, about 1 ms to about 20 ms, about 1 ms to about 30 ms, about 1 ms to about 40 ms, about 1 ms to about 50 ms, about 1 ms to about 100 ms, about 1 ms to about 200 ms, about 5 ms to about 10 ms, about 5 ms to about 20 ms, about 5 ms to about 30 ms, about 5 ms to about 40 ms, about 5 ms to about 50 ms, about 5 ms to about 100 ms, about 5 ms to about 200 ms, about 10 ms to about 20 ms, about 10 ms to about 30 ms, about 10 ms to about 40 ms, about 10 ms to about 50 ms, about 10 ms to about 100 ms, about 10 ms to about 200 ms, about 20 ms to about 30 ms, about 20 ms to about 40 ms, about 20 ms to about 50 ms, about 20 ms to about 100 ms, about 20 ms to about 200 ms, about 30 ms to about 40 ms, about 30 ms to about 50 ms, about 30 ms to about 100 ms, about 30 ms to about 200 ms, about 40 ms to about 50 ms, about 40 ms to about 100 ms, about 40 ms to about 200 ms, about 50 ms to about 100 ms, about 50 ms to about 200 ms, or about 100 ms to about 200 ms. In some cases, the pulse interval is about 1 ms, about 5 ms, about 10 ms, about 20 ms, about 30 ms, about 40 ms, about 50 ms, about 100 ms, or about 200 ms. In some cases, the pulse interval is at least about 1 ms, about 5 ms, about 10 ms, about 20 ms, about 30 ms, about 40 ms, about 50 ms, or about 100 ms. In some cases, the pulse interval is at most about 5 ms, about 10 ms, about 20 ms, about 30 ms, about 40 ms, about 50 ms, about 100 ms, or about 200 ms.

In some cases, a voltage cycle or series comprises about 1 pulse to about 200 pulses. In some cases, a voltage cycle comprises about 1 pulse to about 10 pulses, about 1 pulse to about 30 pulses, about 1 pulse to about 50 pulses, about 1 pulse to about 70 pulses, about 1 pulse to about 100 pulses, about 1 pulse to about 200 pulses, about 10 pulses to about 30 pulses, about 10 pulses to about 50 pulses, about 10 pulses to about 70 pulses, about 10 pulses to about 100 pulses, about 10 pulses to about 200 pulses, about 30 pulses to about 50 pulses, about 30 pulses to about 70 pulses, about 30 pulses to about 100 pulses, about 30 pulses to about 200 pulses, about 50 pulses to about 70 pulses, about 50 pulses to about 100 pulses, about 50 pulses to about 200 pulses, about 70 pulses to about 100 pulses, about 70 pulses to about 200 pulses, or about 100 pulses to about 200 pulses. In some cases, a voltage cycle comprises about 1 pulse, about 10 pulses, about 30 pulses, about 50 pulses, about 70 pulses, about 100 pulses, or about 200 pulses. In some cases, a voltage cycle comprises at least about 1 pulse, about 10 pulses, about 30 pulses, about 50 pulses, about 70 pulses, or about 100 pulses. In some cases, a voltage cycle comprises at most about 10 pulses, about 30 pulses, about 50 pulses, about 70 pulses, about 100 pulses, or about 200 pulses.

In some cases, a series or cycle of voltage pulses are applied as a waveform. In some cases, a series or cycle of voltage pulses are applied as a square waveform, a sinusoidal waveform, a triangular waveform, a sawtooth waveform, or a combination thereof.

In some cases, the transfection reagents to be electroporated have small molecular weight. In some cases, the transfection reagents to be electroporated have large molecular weight. In some cases, the molecular weight of the transfection reagents to be electroporated is about 100 g/mol to about 1000 g/mol. In some cases, the molecular weight of the transfection reagents to be electroporated is about 1000 g/mol to about 2000 g/mol. In some cases, the molecular weight of the transfection reagents to be electroporated is about 2000 g/mol to about 3000 g/mol. In some cases, the molecular weight of the transfection reagents to be electroporated is about 3000 g/mol to about 4000 g/mol. In some cases, the molecular weight of the transfection reagents to be electroporated is about 4000 g/mol to about 5000 g/mol. In some cases, the molecular weight of the transfection reagents to be electroporated is about 5000 g/mol to about 7500 g/mol. In some cases, the molecular weight of the transfection reagents to be electroporated is about 7500 g/mol to about 10000 g/mol. In some cases, the transfection reagents to be electroporated comprise DNAs, RNAs, proteins, other charged biomolecules, and/or charged molecules. In some specific cases, the transfection reagents to be electroporated comprise plasmid DNAs. In some specific cases, the transfection reagents to be electroporated comprise siRNAs. In some specific cases, the transfection reagents to be electroporated comprise mRNA, encapsulated in nanolipid particles or not. In some specific cases, the transfection reagents to be electroporated comprise miRNAs. In some specific cases, the transfection reagents to be electroporated comprise shRNAs. In some specific cases, the transfection reagents to be electroporated comprise a small-molecule drug. In some specific cases, the transfection reagents to be electroporated comprise polypeptides. In some specific cases, the transfection reagents to be electroporated comprise antibodies. In some specific cases, the transfection reagents to be electroporated comprise a combination of the above-described reagents.

In some cases, the method of using the electroporation system described herein further comprises removing cells from the system, such as by removing cells from a cell chamber within a tube described herein. In some cases, the method comprises removing cells from a cell chamber such as a cell chamber described herein, e.g., in Section I. In some cases, the cells are removed by a syringe (see e.g., 160 in FIG. 1A). In some cases, the cells are removed by a serological pipette. In some specific cases, the cells are re-suspended in the suspension before being removed. In some cases, the cells are transferred from the centrifuge tube described in Section I to a sterile container an incubator for recovery and further growth. In some cases, the cells are transferred to be in their growth media.

In some cases, the method of using the electroporation system described herein described herein further comprises repeating the above steps, including (1) providing a new suspension comprising a plurality of cells after removing the previous batch of cells, (2) applying a centrifugal force to the plurality of cells, such that at least a portion of the plurality of cells is pressed against the first surface of the electroporation chip; (3) providing an electrical voltage across the electroporation chip as the centrifugal force is applied to the plurality of cells; and optionally (4) the plurality of cells are re-suspended, removed and incubated for recovery and further growth. In some cases, the repeating of the above steps comprises varying the type of cells being electroporated. In some cases, the repeating of the above steps comprises varying the kind of buffer used. In some cases, the repeating of the above steps comprises varying the centrifuge speed. In some cases, the repeating of the above steps comprises varying the duration of the centrifuge. In some cases, the repeating of the above steps comprises varying voltage of the electrical current. In some cases, the repeating of the above steps comprises varying duration of the electrical current. In some cases, the repeating of the above steps comprises varying the pulse length. In some cases, the repeating of the above steps comprises varying the pulse interval. In some cases, the repeating of the above steps comprises varying the waveform type of the electrical current. In some cases, the repeating of the above steps comprises varying the transfection reagents to be electroporated. In some cases, the repeating of the above steps comprises a combination of all or part of the above-described parameters.

### IV. Methods of Electroporating Cells

The methods provided herein include methods of electroporating cells (e.g., adherent cells, suspension cells) while subjecting the cells to a centrifugal force. Described herein, in some embodiments, is a method of electroporating cells comprising introducing a suspension comprising a plurality of cells (e.g., suspension cells, adherent cells) to an electroporation chip; applying a centrifugal force to the plurality of cells, such that at least a portion of the plurality of cells is pressed against a surface of the electroporation chip; and providing an electrical voltage across the electroporation chip as the centrifugal force is applied to the plurality of cells. Described herein, in some embodiments, is a method of electroporating cells, comprising providing a suspension comprising a plurality of cells (e.g., suspension cells, adherent cells) to the first side of the electroporation chip; applying a centrifugal force to the plurality of cells, such that at least a portion of the plurality of cells is pressed against the first surface of the electroporation chip; and providing an electrical voltage across the electroporation chip as the centrifugal force is applied to the plurality of cells. In some embodiments, the cells are suspension cells.

In some cases, the method of electroporating cells comprises providing a suspension comprising a plurality of cells to an enclosed space. In some cases, the method of electroporating cells comprises providing a suspension comprising a plurality of cells to an enclosed space that is sterile. In some cases, by providing the cells to a sterile enclosed space, the cells can be later cultured in another sterile environment. In some cases, extracellular vesicles (e.g., exosomes, microvesicle, apoptotic body, etc.) are collected from the media of the cultured cells. In some cases, the method of electroporating cells comprises providing a suspension comprising a plurality of cells to a first surface of an electroporation chip as described herein. In some cases, the suspension comprising a plurality of cells is provided within the cell chamber as described herein. In some specific cases, as depicted in FIG. 1A, the suspension comprising a plurality of cells is provided within the cell chamber 164 that is formed by a side of a stabilizer 168 and a first side of the electroporation chip 150.

In some cases, the suspension comprising a plurality of cells is provided through a sterile structure that is connected between the exterior of the centrifuge tube and the cell chamber described herein. In some specific cases, the suspension comprising a plurality of cells is provided through a syringe, e.g., a syringe 160 as depicted in FIG. 1A. In some specific cases, the suspension comprising a plurality of cells is provided through a serological pipette.

In some cases, the suspension comprising a plurality of cells comprises a saline-based buffer that is suitable for electroporation. In some cases, the suspension comprising a plurality of cells comprises a phosphate-based buffer that is suitable for electroporation. In some cases, the suspension comprising a plurality of cells comprises a HEPES-based buffer that is suitable for electroporation. In some cases, the suspension comprising a plurality of cells comprises a cell-culture-media-based buffer that is suitable for electroporation.

In some cases, the plurality of cells within the suspension are of a certain cell type; in some cases, the plurality of cells are a mixture of different cell types (e.g., PBMCs). In particular embodiments, the suspension comprises suspension cells, e.g., cells that do not typically adhere to a plate during cell culture. In some cases, the suspense comprises adherent cells. In some cases, the suspension comprises a plurality of eukaryotic cells. In some specific cases, the suspension comprises a plurality of mammalian cells. In some particular cases, the suspension comprises a plurality of human cells. In some cases, the suspension comprises non-human cells (e.g., rodent cells, primate cells, etc.). In some cases, the suspension comprises a plurality of cells from a cell line. In some cases, the suspension comprises a plurality of primary cells, e.g., primary cells obtained from dissecting a target tissue or organ, or primary cells obtained from blood (e.g., PBMCs, PBLs, monocytes, macrophages, dendritic cells, lymphocytes, myeloid cells, stem cells, hematopoietic stem cells).

In some cases, the suspension comprises a plurality of cells from a cell line. In some cases, the plurality of cells from a cell line is from a suspension cell line or an adherent cell line. In some specific cases, the cell line described herein is of a myeloma origin. In some specific cases, the cell line described herein is of a lymphoma origin. In some specific cases, the cell line described herein is of a leukemia origin. In some cases, the cell line described herein has an epithelial morphology. In some cases, the cell line described herein has an endothelial morphology. In some cases, the cell line described herein has a neuronal morphology. In some cases, the cell line described herein has an endothelial morphology. In some specific cases, the plurality of eukaryotic cells comprise human lung. In some specific cases, the plurality of eukaryotic cells comprise human cervix. In some specific cases, the plurality of eukaryotic cells comprise African green monkey kidney. In some specific cases, the plurality of eukaryotic cells comprise mouse embryo. In some specific cases, the plurality of eukaryotic cells comprise mouse connective tissue. In some specific cases, the plurality of eukaryotic cells comprise Chinese hamster ovary. In some specific cases, the plurality of eukaryotic cells comprise Syrian hamster kidney. In some specific cases, the plurality of eukaryotic cells comprise human kidney. In some specific cases, the plurality of eukaryotic cells comprise human liver. In some specific cases, the plurality of eukaryotic cells comprise bovine aorta. In some specific cases, the plurality of eukaryotic cells comprise human neuroblastoma. In some specific cases, the plurality of eukaryotic cells comprise mouse myeoloa. In some specific cases, the plurality of eukaryotic cells comprise human hystiocytic lymphoma. In some specific cases, the plurality of eukaryotic cells comprise human leukemia. In some specific cases, the plurality of eukaryotic cells comprise mouse B-cell lymphoma. In some specific cases, the plurality of eukaryotic cells comprise mouse lymphoma. In some specific cases, the plurality of eukaryotic cells comprise human myeloma. In some specific cases, the plurality of eukaryotic cells comprise human T-cell leukemia. In some specific cases, the plurality of eukaryotic cells comprise human monocyte leukemia. In some specific cases, the plurality of eukaryotic cells comprise mouse embryonic fibroblasts (MEF). In some specific cases, the plurality of eukaryotic cells comprise human embryonic fibroblasts (HEF). In some specific cases, the plurality of eukaryotic cells comprise dendritic cells. In some specific cases, the plurality of eukaryotic cells comprise mesenchymal stem cells. In some specific cases, the plurality of eukaryotic cells comprise bone marrow-derived dendritic cells. In some specific cases, the plurality of eukaryotic cells comprise bone marrow derived stromal cells. In some specific cases, the plurality of eukaryotic cells comprise adipose stromal cells. In some specific cases, the plurality of eukaryotic cells comprise enucleated cells. In some specific cases, the plurality of eukaryotic cells comprise neural stem cells. In some specific cases, the plurality of eukaryotic cells comprise immature dendritic cells. In some specific cases, the plurality of eukaryotic cells comprise immune cells. In some specific cases, the plurality of eukaryotic cells comprise NS0. In some specific cases, the plurality of eukaryotic cells comprise U937. In some specific cases, the plurality of eukaryotic cells comprise HL60. In some specific cases, the plurality of eukaryotic cells comprise WEHI231. In some specific cases, the plurality of eukaryotic cells comprise YAC1. In some specific cases, the plurality of eukaryotic cells comprise U266B1. In some specific cases, the plurality of eukaryotic cells comprise Jurkat. In some specific cases, the plurality of eukaryotic cells comprise THP-1. In some specific cases, the plurality of eukaryotic cells comprise MRC-5. In some specific cases, the plurality of eukaryotic cells comprise HeLa. In some specific cases, the plurality of eukaryotic cells comprise HEK-293. In some specific cases, the plurality of eukaryotic cells comprise HepG2. In some specific cases, the plurality of eukaryotic cells comprise SH-SY5Y. In some specific cases, the plurality of eukaryotic cells comprise MCF-7. In some specific cases, the plurality of eukaryotic cells comprise Sf9, Vero, NIH3T3, L929, CHO, BHK-21, Cos7, or BAE-1.

In some cases, the suspension described herein comprises a plurality of cells with a low elastic modulus (effective Young's modulus). In some cases, the suspension described herein comprises a plurality of cells with a Poisson ratio of about 0.5. In some cases, the suspension described herein comprises a plurality of cells with a Poisson ratio of about 0.4. In some cases, the suspension described herein comprises a plurality of cells with a Poisson ratio of about 0.3.

In some cases, the suspension comprising a plurality of cells at about 1×10² to about 1×10³ cells/mL. In other cases, the suspension comprising a plurality of cells at about 1×10³ to about 1×10⁴ cells/mL. In other cases, the suspension comprising a plurality of cells at about 1×10⁴ to about 1×10⁵ cells/mL. In other cases, the suspension comprising a plurality of cells at about 1×10⁵ to about 1×10⁶ cells/mL. In other cases, the suspension comprising a plurality of cells at about 1×10⁶ to about 1×10⁷ cells/mL.

In some cases, the methods described herein comprises applying centrifugal force to the plurality of cells, such that at least a portion of the plurality of cells is pressed against the first surface of the electroporation chip.

In some cases, while an electric field is being applied across an electroporation chip of a centrifuge tube, as described herein, the centrifuge rotates to produce a relative centrifugal force (RCF) of about 1 to 3000 g. In some cases, the centrifuge rotates at about 1 g to about 50 g, about 1 g to about 100 g, about 1 g to about 300 g, about 1 g to about 500 g, about 1 g to about 700 g, about 1 g to about 1,000 g, about 1 g to about 1,500 g, about 1 g to about 2,000 g, about 1 g to about 2,500 g, about 1 g to about 3,000 g, about 50 g to about 100 g, about 50 g to about 300 g, about 50 g to about 500 g, about 50 g to about 700 g, about 50 g to about 1,000 g, about 50 g to about 1,500 g, about 50 g to about 2,000 g, about 50 g to about 2,500 g, about 50 g to about 3,000 g, about 100 g to about 300 g, about 100 g to about 500 g, about 100 g to about 700 g, about 100 g to about 1,000 g, about 100 g to about 1,500 g, about 100 g to about 2,000 g, about 100 g to about 2,500 g, about 100 g to about 3,000 g, about 300 g to about 500 g, about 300 g to about 700 g, about 300 g to about 1,000 g, about 300 g to about 1,500 g, about 300 g to about 2,000 g, about 300 g to about 2,500 g, about 300 g to about 3,000 g, about 500 g to about 700 g, about 500 g to about 1,000 g, about 500 g to about 1,500 g, about 500 g to about 2,000 g, about 500 g to about 2,500 g, about 500 g to about 3,000 g, about 700 g to about 1,000 g, about 700 g to about 1,500 g, about 700 g to about 2,000 g, about 700 g to about 2,500 g, about 700 g to about 3,000 g, about 1,000 g to about 1,500 g, about 1,000 g to about 2,000 g, about 1,000 g to about 2,500 g, about 1,000 g to about 3,000 g, or about 1,500 g to about 2,000 g, about 1,500 g to about 2,500 g, about 1,500 g to about 3,000 g, about 2,000 g to about 2,500 g, about 2,000 g to about 3,000 g, or about 2,500 g to about 3,000 g. In some cases, the centrifuge rotates at about 1 g, about 50 g, about 100 g, about 300 g, about 500 g, about 700 g, about 1,000 g, about 1,500 g, or about 2,000 g. In some cases, the centrifuge rotates at least about 1 g, about 50 g, about 100 g, about 300 g, about 500 g, about 700 g, about 1,000 g, or about 1,500 g. In some cases, the centrifuge rotates at most about 50 g, about 100 g, about 300 g, about 500 g, about 700 g, about 1,000 g, about 1,500 g, about 2,000 g, about 2,500 g, about 3,000 g.

In some cases, the cells are centrifuged (or spun) at a certain speed. In some cases, the cells that are electroporated are centrifuged with a speed of 700 rpm to 1200 rpm or from about 700 rpm to about 1200 rpm. In some cases, the cells that are electroporated are centrifuged with a speed of at least 650 rpm, at least 700 rpm, at least 750 rpm, or at least 800 rpm. In some cases, the cells that are electroporated are centrifuged with a speed of at least 550 rpm, at least 600 rpm, at least 650 rpm, at least 700 rpm, at least 750 rpm, or at least 800 rpm. In some cases, the cells that are electroporated are centrifuged with a speed of at least 500, 600, 700, 800, 900, 1000, 1100, 1200, 1300, 1400, or 1500 rpm. In some cases, the cells that are electroporated are centrifuged with a speed of not more than 1200 rpm or not more than about 1200 rpm. In some cases, the cells that are electroporated are centrifuged with a speed not more than 500, 600, 700, 800, 900, 1000, 1100, 1200, 1300, 1400, 1500, 1600, 1700, 1800, 1900, 2000, 2100, 2200, 2300, 2400, or 2500 rpm. In some cases, the cells that are electroporated are centrifuged with a speed of about 500, 600, 700, 800, 900, 1000, 1100, 1200, 1300, 1400, 1500, 1600, 1700, 1800, 1900, 2000, 2100, 2200, 2300, 2400, or 2500 rpm. In some cases, the cells that are electroporated are centrifuged with a speed of about 500 -2500 rpm, 500-2000 rpm, 500-1500 rpm, or 500-1000 rpm. In some cases, the cells that are electroporated are centrifuged with a speed of about 700-1200 rpm, 800-1200 rpm, 900-1200 rpm, 1000-1200 rpm, or 1100-1200 rpm. In some cases, the cells that are electroporated are centrifuged with a speed of about 1200-1500 rpm, 1300-1500 rpm, 1400-1500 rpm, 1200-1400 rpm, or 1200-1300 rpm.

In some particular cases, the cells that are electroporated are centrifuged with a speed of more than 700 rpm. In some particular cases, the cells that are electroporated are centrifuged with a speed of not more than about 1200 rpm. In some particular cases, the cells that are electroporated are centrifuged with a speed of about 1200-1500 rpm. In some particular cases, the cells that are electroporated are centrifuged with a speed of about 1500 rpm.

In some cases, while an electric field is being applied across an electroporation chip of a centrifuge tube, as described herein, the centrifuge rotates for a duration of 1 minute to an hour to complete an electroporation cycle. In some cases, the centrifuge rotates for about 1 minute to about 120 minutes. In some cases, the centrifuge rotates for about 1 minute to about 3 minutes, about 1 minute to about 5 minutes, about 1 minute to about 7 minutes, about 1 minute to about 10 minutes, about 1 minute to about 15 minutes, about 1 minute to about 30 minutes, about 1 minute to about 45 minutes, about 1 minute to about 60 minutes, about 1 minute to about 120 minutes, about 3 minutes to about 5 minutes, about 3 minutes to about 7 minutes, about 3 minutes to about 10 minutes, about 3 minutes to about 15 minutes, about 3 minutes to about 30 minutes, about 3 minutes to about 45 minutes, about 3 minutes to about 60 minutes, about 3 minutes to about 120 minutes, about 5 minutes to about 7 minutes, about 5 minutes to about 10 minutes, about 5 minutes to about 15 minutes, about 5 minutes to about 30 minutes, about 5 minutes to about 45 minutes, about 5 minutes to about 60 minutes, about 5 minutes to about 120 minutes, about 7 minutes to about 10 minutes, about 7 minutes to about 15 minutes, about 7 minutes to about 30 minutes, about 7 minutes to about 45 minutes, about 7 minutes to about 60 minutes, about 7 minutes to about 120 minutes, about 10 minutes to about 15 minutes, about 10 minutes to about 30 minutes, about 10 minutes to about 45 minutes, about 10 minutes to about 60 minutes, about 10 minutes to about 120 minutes, about 15 minutes to about 30 minutes, about 15 minutes to about 45 minutes, about 15 minutes to about 60 minutes, about 15 minutes to about 120 minutes, about 30 minutes to about 45 minutes, about 30 minutes to about 60 minutes, about 30 minutes to about 120 minutes, about 45 minutes to about 60 minutes, about 45 minutes to about 120 minutes, or about 60 minutes to about 120 minutes. In some cases, the centrifuge rotates for about 1 minute, about 3 minutes, about 5 minutes, about 7 minutes, about 10 minutes, about 15 minutes, about 30 minutes, about 45 minutes, about 60 minutes, or about 120 minutes. In some cases, the centrifuge rotates for at least about 1 minute, about 3 minutes, about 5 minutes, about 7 minutes, about 10 minutes, about 15 minutes, about 30 minutes, about 45 minutes, or about 60 minutes. In some cases, the centrifuge rotates for at most about 3 minutes, about 5 minutes, about 7 minutes, about 10 minutes, about 15 minutes, about 30 minutes, about 45 minutes, about 60 minutes, or about 120 minutes.

In some cases, at least 10% of the cells are pressed against the first surface of the electroporation chip. In other cases, at least 20% of the cells are pressed against the first surface of the electroporation chip. In other cases, at least 30% of the cells are pressed against the first surface of the electroporation chip. In other cases, at least 40% of the cells are pressed against the first surface of the electroporation chip. In other cases, at least 50% of the cells are pressed against the first surface of the electroporation chip. In other cases, at least 60% of the cells are pressed against the first surface of the electroporation chip. In other cases, at least 70% of the cells are pressed against the first surface of the electroporation chip. In other cases, at least 80% of the cells are pressed against the first surface of the electroporation chip. In other cases, at least 90% of the cells are pressed against the first surface of the electroporation chip. In other cases, at least 100% of the cells are pressed against the first surface of the electroporation chip.

In some cases, the cells that are pressed against the first surface of the electroporation chip are elongated to have a longer axis of about 110% of their original diameter. In other cases, the cells that are pressed against the first surface of the electroporation chip are elongated to have a longer axis of about 120% of their original diameter. In other cases, the cells that are pressed against the first surface of the electroporation chip are elongated to have a longer axis of about 130% of their original diameter. In other cases, the cells that are pressed against the first surface of the electroporation chip are elongated to have a longer axis of about 140% of their original diameter. In other cases, the cells that are pressed against the first surface of the electroporation chip are elongated to have a longer axis of about 150% of their original diameter. In other cases, the cells that are pressed against the first surface of the electroporation chip are elongated to have a longer axis of about 160%, about 170%, about 180%, about 190%, about 200%, about 250%, about 300%, about 400%, about 500% of their original diameter.

In some cases, the cells that are pressed against the first surface of the electroporation chip are flattened to have a shorter axis of about 90% of their original diameter. In other cases, the cells that are pressed against the first surface of the electroporation chip are flattened to have a shorter axis of about 80% of their original diameter. In other cases, the cells that are pressed against the first surface of the electroporation chip are flattened to have a shorter axis of about 70% of their original diameter. In other cases, the cells that are pressed against the first surface of the electroporation chip are flattened to have a shorter axis of about 60% of their original diameter. In other cases, the cells that are pressed against the first surface of the electroporation chip are flattened to have a shorter axis of about 50% of their original diameter. In other cases, the cells that are pressed against the first surface of the electroporation chip are flattened to have a shorter axis of about 40%, about 30%, about 20%, or about 10% of their original diameter.

In some cases, the cells that are pressed have an increased contact area with the first surface of the electroporation chip by about 10%. In other cases, the cells that are pressed have an increased contact area with the first surface of the electroporation chip by about 20%. In other cases, the cells that are pressed have an increased contact area with the first surface of the electroporation chip by about 30%. In other cases, the cells that are pressed have an increased contact area with the first surface of the electroporation chip by about 40%. In other cases, the cells that are pressed have an increased contact area with the first surface of the electroporation chip by about 50%. In other cases, the cells that are pressed have an increased contact area with the first surface of the electroporation chip by about 60%, about 70%, about 80%, about 90%, about 100%, about 150%, about 200%, about 250%, or about 300%.

In some cases, the gap between the cells that are pressed against the first surface of the electroporation chip and the nearest pores of the electroporation chip is reduced by about 10%, about 20%, about 30%, about 40%, about 50%, about 60%, about 70%, about 80%, or about 90%.

In some cases, the method of electroporating cells comprises providing an electrical voltage across the electroporation chip as the centrifugal force is applied to the plurality of cells. In specific cases, the electrical voltage is provided during the centrifugal force is applied. In specific cases, the electrical voltage is provided after the centrifugal force is applied. In specific cases, the centrifugal force is applied first before the electrical voltage is provided, then the centrifugal force is applied during the electrical voltage is provided. The current described herein is applied to a circuit comprising the first electrode 130 and the second electrode 135 to produce an electric field. In some cases, the current applied to the first electrode 130 and the second electrode 135 at a voltage/distance between two electrodes of 0.5 V/cm to 1000V/cm, including increments therein. In some cases, the voltage is applied at as a pulse. In some cases, the pulse length is 1 to 50 milliseconds (ms) including increments therein. In some cases, the voltage is applied as a series of pulses. In some cases, the series of pulses comprises 1 to 100 pulses. In some cases, the pulses are applied as a square wave signal. In some cases, the duration between pulses (no voltage applied, pulse interval) is approximately equal to the selected pulse duration. For example, if a pulse length of 10 ms is utilized, the pulse cycle includes a voltage application for 10 ms followed by a period of no voltage application for 10 ms. In other cases, the pulse interval is longer than the selected pulse duration. For example, if a pulse length of 10 ms is utilized, the pulse cycle includes a voltage application for 10 ms followed by a period of no voltage application for 100 ms. In other cases, the pulse interval is shorter than the selected pulse duration.

In some cases, the applied voltage/distance between two electrodes is about 0.5 V/cm to about 1,000 V/cm. In some cases, the applied voltage/distance between the two electrodes is about 0.5 V/cm to about 1 V/cm, about 0.5 V/cm to about 100 V/cm, about 0.5 V/cm to about 300 V/cm, about 0.5 V/cm to about 500 V/cm, about 0.5 V/cm to about 800 V/cm, about 0.5 V/cm to about 1,000 V/cm, about 1 V/cm to about 100 V/cm, about 1 V/cm to about 300 V/cm, about 1 V/cm to about 500 V/cm, about 1 V/cm to about 800 V/cm, about 1 V/cm to about 1,000 V/cm, about 100 V/cm to about 300 V/cm, about 100 V/cm to about 500 V/cm, about 100 V/cm to about 800 V/cm, about 100 V/cm to about 1,000 V/cm, about 300 V/cm to about 500 V/cm, about 300 V/cm to about 800 V/cm, about 300 V/cm to about 1,000 V/cm, about 500 V/cm to about 800 V/cm, about 500 V/cm to about 1,000 V/cm, or about 800 V/cm to about 1,000 V/cm. In some cases, the applied voltage/distance between the two electrodes is about 0.5 V/cm, about 1 V/cm, about 100 V/cm, about 300 V/cm, about 500 V/cm, about 800 V/cm, or about 1,000 V/cm. In some cases, the applied voltage is at least about 0.5 V/cm, about 1 V/cm, about 100 V/cm, about 300 V/cm, about 500 V/cm, or about 800 V/cm. In some cases, the applied voltage/distance between the two electrodes is at most about 1 V/cm, about 100 V/cm, about 300 V/cm, about 500 V/cm, about 800 V/cm, or about 1,000 V/cm.

In some cases, a pulse length is about 1 ms to about 50 ms. In some cases, a pulse duration is about 1 ms to about 5 ms, about 1 ms to about 10 ms, about 1 ms to about 15 ms, about 1 ms to about 20 ms, about 1 ms to about 30 ms, about 1 ms to about 50 ms, about 5 ms to about 10 ms, about 5 ms to about 15 ms, about 5 ms to about 20 ms, about 5 ms to about 30 ms, about 5 ms to about 50 ms, about 10 ms to about 15 ms, about 10 ms to about 20 ms, about 10 ms to about 30 ms, about 10 ms to about 50 ms, about 15 ms to about 20 ms, about 15 ms to about 30 ms, about 15 ms to about 50 ms, about 20 ms to about 30 ms, about 20 ms to about 50 ms, or about 30 ms to about 50 ms. In some cases, a pulse duration is about 1 ms, about 5 ms, about 10 ms, about 15 ms, about 20 ms, about 30 ms, or about 50 ms. In some cases, a pulse duration is at least about 1 ms, about 5 ms, about 10 ms, about 15 ms, about 20 ms, or about 30 ms. In some cases, a pulse duration is at most about 5 ms, about 10 ms, about 15 ms, about 20 ms, about 30 ms, or about 50 ms.

In some cases, the pulse interval is about 1 ms to about 200 ms. In some cases, the pulse interval is about 1 ms to about 5 ms, about 1 ms to about 10 ms, about 1 ms to about 20 ms, about 1 ms to about 30 ms, about 1 ms to about 40 ms, about 1 ms to about 50 ms, about 1 ms to about 100 ms, about 1 ms to about 200 ms, about 5 ms to about 10 ms, about 5 ms to about 20 ms, about 5 ms to about 30 ms, about 5 ms to about 40 ms, about 5 ms to about 50 ms, about 5 ms to about 100 ms, about 5 ms to about 200 ms, about 10 ms to about 20 ms, about 10 ms to about 30 ms, about 10 ms to about 40 ms, about 10 ms to about 50 ms, about 10 ms to about 100 ms, about 10 ms to about 200 ms, about 20 ms to about 30 ms, about 20 ms to about 40 ms, about 20 ms to about 50 ms, about 20 ms to about 100 ms, about 20 ms to about 200 ms, about 30 ms to about 40 ms, about 30 ms to about 50 ms, about 30 ms to about 100 ms, about 30 ms to about 200 ms, about 40 ms to about 50 ms, about 40 ms to about 100 ms, about 40 ms to about 200 ms, about 50 ms to about 100 ms, about 50 ms to about 200 ms, or about 100 ms to about 200 ms. In some cases, the pulse interval is about 1 ms, about 5 ms, about 10 ms, about 20 ms, about 30 ms, about 40 ms, about 50 ms, about 100 ms, or about 200 ms. In some cases, the pulse interval is at least about 1 ms, about 5 ms, about 10 ms, about 20 ms, about 30 ms, about 40 ms, about 50 ms, or about 100 ms. In some cases, the pulse interval is at most about 5 ms, about 10 ms, about 20 ms, about 30 ms, about 40 ms, about 50 ms, about 100 ms, or about 200 ms.

In some cases, a voltage cycle or series comprises about 1 pulse to about 200 pulses. In some cases, a voltage cycle comprises about 1 pulse to about 10 pulses, about 1 pulse to about 30 pulses, about 1 pulse to about 50 pulses, about 1 pulse to about 70 pulses, about 1 pulse to about 100 pulses, about 1 pulse to about 200 pulses, about 10 pulses to about 30 pulses, about 10 pulses to about 50 pulses, about 10 pulses to about 70 pulses, about 10 pulses to about 100 pulses, about 10 pulses to about 200 pulses, about 30 pulses to about 50 pulses, about 30 pulses to about 70 pulses, about 30 pulses to about 100 pulses, about 30 pulses to about 200 pulses, about 50 pulses to about 70 pulses, about 50 pulses to about 100 pulses, about 50 pulses to about 200 pulses, about 70 pulses to about 100 pulses, about 70 pulses to about 200 pulses, or about 100 pulses to about 200 pulses. In some cases, a voltage cycle comprises about 1 pulse, about 10 pulses, about 30 pulses, about 50 pulses, about 70 pulses, about 100 pulses, or about 200 pulses. In some cases, a voltage cycle comprises at least about 1 pulse, about 10 pulses, about 30 pulses, about 50 pulses, about 70 pulses, or about 100 pulses. In some cases, a voltage cycle comprises at most about 10 pulses, about 30 pulses, about 50 pulses, about 70 pulses, about 100 pulses, or about 200 pulses.

In some cases, a series or cycle of voltage pulses are applied as a waveform. In some cases, a series or cycle of voltage pulses are applied as a square waveform, a sinusoidal waveform, a triangular waveform, a sawtooth waveform, or a combination thereof.

In some cases, the transfection reagents to be electroporated have small molecular weight. In some cases, the transfection reagents to be electroporated have large molecular weight. In some cases, the molecular weight of the transfection reagents to be electroporated is about 100g/mol to about 1000 g/mol. In some cases, the molecular weight of the transfection reagents to be electroporated is about 1000 g/mol to about 2000 g/mol. In some cases, the molecular weight of the transfection reagents to be electroporated is about 2000 g/mol to about 3000 g/mol. In some cases, the molecular weight of the transfection reagents to be electroporated is about 3000 g/mol to about 4000 g/mol. In some cases, the molecular weight of the transfection reagents to be electroporated is about 4000 g/mol to about 5000 g/mol. In some cases, the molecular weight of the transfection reagents to be electroporated is about 5000 g/mol to about 7500 g/mol. In some cases, the molecular weight of the transfection reagents to be electroporated is about 7500 g/mol to about 10000 g/mol. In some cases, the transfection reagents to be electroporated comprise DNAs, RNAs, proteins, other charged biomolecules, and/or charged molecules. In some specific cases, the transfection reagents to be electroporated comprise plasmid DNAs. In some specific cases, the transfection reagents to be electroporated comprise siRNAs. In some specific cases, the transfection reagents to be electroporated comprise mRNA, encapsulated in nanolipid particles or not. In some specific cases, the transfection reagents to be electroporated comprise miRNAs. In some specific cases, the transfection reagents to be electroporated comprise shRNAs. In some specific cases, the transfection reagents to be electroporated comprise a small-molecule drug. In some specific cases, the transfection reagents to be electroporated comprise polypeptides. In some specific cases, the transfection reagents to be electroporated comprise antibodies. In some specific cases, the transfection reagents to be electroporated comprise a combination of the above-described reagents.

In some cases, the method of electroporating cells described herein further comprises removing cells from the cell chamber described herein. In some cases, the cells are removed by a syringe (see e.g., 160 in FIG. 1A). In some cases, the cells are removed by a serological pipette. In some specific cases, the cells are re-suspended in the suspension before being removed. In some cases, the cells are transferred from the centrifuge tube described in Section I to a sterile container an incubator for recovery and further growth. In some cases, the cells are transferred to be in their growth media.

In some cases, the method of electroporating cells described herein further comprises repeating the above steps, including (1) providing a new suspension comprising a plurality of cells after removing the previous batch of cells, (2) applying a centrifugal force to the plurality of cells, such that at least a portion of the plurality of cells is pressed against the first surface of the electroporation chip; (3) providing an electrical voltage across the electroporation chip as the centrifugal force is applied to the plurality of cells; and optionally (4) the plurality of cells are re-suspended, removed and incubated for recovery and further growth. In some cases, the repeating of the above steps comprises varying the type of cells being electroporated. In some cases, the repeating of the above steps comprises varying the kind of buffer used. In some cases, the repeating of the above steps comprises varying the centrifuge speed. In some cases, the repeating of the above steps comprises varying the duration of the centrifuge. In some cases, the repeating of the above steps comprises varying voltage of the electrical current. In some cases, the repeating of the above steps comprises varying duration of the electrical current. In some cases, the repeating of the above steps comprises varying the pulse length. In some cases, the repeating of the above steps comprises varying the pulse interval. In some cases, the repeating of the above steps comprises varying the waveform type of the electrical current. In some cases, the repeating of the above steps comprises varying the transfection reagents to be electroporated. In some cases, the repeating of the above steps comprises a combination of all or part of the above-described parameters.

Described herein is a method of electroporating cells comprising: (1) providing a suspension comprising a plurality of cells adjacent to an electroporation chip; (2) elongating the plurality of cells, such that at least a portion of the plurality of cells is pressed against a first surface of the electroporation chip; and (3) providing an electrical current across the electroporation chip. In some cases, elongating the plurality of cells comprises natural sedimentation. In some cases, elongating the plurality of cells comprises a form of an accelerated sedimentation. In some cases, elongating the plurality of cells comprises applying a force the plurality of cells against the first surface of the electroporation chip.

Described herein is a method of electroporating cells comprising: (1) providing a suspension comprising a plurality of cells adjacent to an electroporation chip; (2) flattening the plurality of cells, such that at least a portion of the plurality of cells is pressed against a first surface of the electroporation chip; and (3) providing an electrical current across the electroporation chip. In some cases, flattening the plurality of cells comprises natural sedimentation. In some cases, flattening the plurality of cells comprises a form of an accelerated sedimentation. In some cases, flattening the plurality of cells comprises applying a force the plurality of cells against the first surface of the electroporation chip.

Described herein is a method of electroporating cells comprising: (1) providing a suspension comprising a plurality of cells adjacent to an electroporation chip; (2) increasing the contact area of at least a portion of the plurality of cells against a first surface of the electroporation chip; and (3) providing an electrical current across the electroporation chip. In some cases, increasing the contact area of the plurality of cells comprises natural sedimentation. In some cases, increasing the contact area of the plurality of cells comprises a form of an accelerated sedimentation. In some cases, increasing the contact area of the plurality of cells comprises applying a force the plurality of cells against the first surface of the electroporation chip.

Described herein is a method of electroporating cells comprising: (1) providing a suspension comprising a plurality of cells adjacent to an electroporation chip; (2) reducing the gap between at least a portion of the plurality of cells and a first surface of the electroporation chip; and (3) providing an electrical current across the electroporation chip. In some cases, reducing the gap comprises natural sedimentation. In some cases, reducing the gap comprises a form of an accelerated sedimentation. In some cases, reducing the gap comprises applying a force the plurality of cells against the first surface of the electroporation chip. In some cases, reducing the gap comprises increasing pores to cells ratio. In some specific cases, reducing the gap comprises increasing the pore density of the electroporation chip. In some cases, reducing the gap comprises reducing the surface of the electroporation chip that is not covered with pores. In some specific cases, reducing the gap comprises increasing pore size.

Described herein is a method of electroporating cells comprising: (1) providing a suspension comprising a plurality of cells adjacent to an electroporation chip; and (2) increasing the transmembrane potential of the plurality of cells without increasing the voltage of an electrical current across the electroporation chip.

### V. Methods of Producing Extracellular Vesicles Using Centrifuge-N/MEP

This disclosure provides methods of producing extracellular vesicles using the centrifuge-N/MEP described herein. Generally, by taking the advantage of the centrifuge-N/MEP described herein, such as high yield and high-throughput delivery, production of extracellular vesicles can be performed more efficiently, as seen in the Example described herein. In some cases, the method of producing extracellular vesicles using the centrifuge-N/MEP provided herein comprises placing extracellular vesicle donor cell (e.g., extracellular-vesicle producing cells) on the electroporation chip, centrifuging, electroporating biomolecules of interest, removing and culturing cells, harvesting released extracellular vesicles, purifying extracellular vesicles. Also provided herein are methods of treating a subject in need of with the extracellular vesicles produced by the method of centrifuge-N/MEP described herein.

The extracellular vesicle donor cells can be any type of cell. In some cases, the extracellular vesicle donor cells are eukaryotic cells (e.g., mammalian cells, human cells, non-human mammalian cells, rodent cells, mouse cells, etc.). In some instances, the extracellular vesicle donor cells are cells from a cell line, stem cells, primary cells, or differentiated cells. In some embodiments, the extracellular vesicle donor cells are primary cells. In some instances, the extracellular vesicle donor cells are mouse embryonic fibroblasts (MEF), human embryonic fibroblasts (HEF), human dermal fibroblasts (HDF), dendritic cells, mesenchymal stem cells, bone marrow-derived dendritic cells, bone marrow derived stromal cells, adipose stromal cells, enucleated cells, neural stem cells, immature dendritic cells, or immune cells. The extracellular vesicle donor cells may be adherent cells. In some cases, the extracellular vesicle donor cells are adherent cells. In some cases, the extracellular vesicle donor cells are suspension cells. In some cases, the extracellular vesicle donor cells are suspension cell lines. In some cases, the extracellular vesicle donor cells are suspension primary cells.

In some cases, a suspension comprising the extracellular vesicle donor cells described herein to a first surface of an electroporation chip as described herein. In some cases, the suspension comprising the extracellular vesicle donor cells described herein is provided within the cell chamber as described herein. In some specific cases, as depicted in FIG. 1A, the suspension comprising the extracellular vesicle donor cells described herein is provided within the cell chamber 164 that is formed by a side of a stabilizer 168 and a first side of the electroporation chip 150.

The transfection reagents can be any type of biomolecules. In some cases, the transfection agents are at least one heterologous polynucleotide such as a vector (e.g., plasmid, DNA). In specific cases, the at least one heterologous polynucleotide encodes at least one polypeptide. In certain cases, the at least one polypeptide is therapeutic. In certain cases, the at least one polypeptide is for targeted delivery of the extracellular vesicle. In certain cases, the at least one polypeptide is both therapeutic and for targeted delivery of the extracellular vesicle. In other cases, the transfection reagents can be a therapeutic compound (e.g., a therapeutic DNA, therapeutic RNA, therapeutic mRNA, therapeutic miRNA, therapeutic tRNA, therapeutic rRNA, therapeutic siRNA, therapeutic shRNA, therapeutic SRP RNA, therapeutic tmRNA, therapeutic gRNA, or therapeutic crRNA), a therapeutic non-coding polynucleotide (e.g., non-coding RNA, IncRNA, piRNA, snoRNA, snRNAs, exRNA, or scaRNA), a drug, or a combination thereof. In other cases, the transfection reagents can be a non-therapeutic compound (e.g., non-therapeutic polynucleotide). In some cases, the transfection reagents loaded in the reservoir under the N/MEP chip.

The centrifuge-N/MEP used in the methods of producing extracellular vesicles are described herein, including loading suspension on the chip, centrifuging, electroporating, removing cells.

As seen in the Example provided herein, the voltage used in the centrifuge-N/MEP for producing extracellular vesicles could affect the efficiency of producing such extracellular vesicles. In some cases, the voltage used in the centrifuge-N/MEP for producing extracellular vesicles can be about 50, 60, 70, 80, or 90 V. In some cases, the voltage used in the centrifuge-N/MEP for producing extracellular vesicles can be about 100V. In some cases, the voltage used in the centrifuge-N/MEP for producing extracellular vesicles can be about 110V. In some cases, the voltage used in the centrifuge-N/MEP for producing extracellular vesicles can be about 120V. In some cases, the voltage used in the centrifuge-N/MEP for producing extracellular vesicles can be about 130V. In some cases, the voltage used in the centrifuge-N/MEP for producing extracellular vesicles can be about 140V. In some cases, the voltage used in the centrifuge-N/MEP for producing extracellular vesicles can be about 150, 160, 170, 180, 190, or 200V. In some cases, the voltage used in the centrifuge-N/MEP for producing extracellular vesicles can be at least about 50, 60, 70, 80, or 90 V. In some cases, the voltage used in the centrifuge-N/MEP for producing extracellular vesicles can be at least about 100V. In some cases, the voltage used in the centrifuge-N/MEP for producing extracellular vesicles can be at least about 110V. In some cases, the voltage used in the centrifuge-N/MEP for producing extracellular vesicles can be at least about 120V. In some cases, the voltage used in the centrifuge-N/MEP for producing extracellular vesicles can be at least about 130V. In some cases, the voltage used in the centrifuge-N/MEP for producing extracellular vesicles can be at least about 140V. In some cases, the voltage used in the centrifuge-N/MEP for producing extracellular vesicles can be at least about 150, 160, 170, 180, 190, or 200V. In some cases, the voltage used in the centrifuge-N/MEP for producing extracellular vesicles can be at most about 50, 60, 70, 80, or 90 V. In some cases, the voltage used in the centrifuge-N/MEP for producing extracellular vesicles can be at most about 100V. In some cases, the voltage used in the centrifuge-N/MEP for producing extracellular vesicles can be at most about 110V. In some cases, the voltage used in the centrifuge-N/MEP for producing extracellular vesicles can be at most about 120V. In some cases, the voltage used in the centrifuge-N/MEP for producing extracellular vesicles can be at most about 130V. In some cases, the voltage used in the centrifuge-N/MEP for producing extracellular vesicles can be at most about 140V. In some cases, the voltage used in the centrifuge-N/MEP for producing extracellular vesicles can be about at most 150, 160, 170, 180, 190, or 200V.

As seen in the Example provided herein, the number of pulses used in the centrifuge-N/MEP for producing extracellular vesicles could affect the efficiency of producing such extracellular vesicles. In some cases, the number of pulses used in the centrifuge-N/MEP for producing extracellular vesicles is about 10 pulses. In some cases, the number of pulses used in the centrifuge-N/MEP for producing extracellular vesicles is about 20 pulses. In some cases, the number of pulses used in the centrifuge-N/MEP for producing extracellular vesicles is about 30 pulses. In some cases, the number of pulses used in the centrifuge-N/MEP for producing extracellular vesicles is about 40 pulses. In some cases, the number of pulses used in the centrifuge-N/MEP for producing extracellular vesicles is at least about 10 pulses. In some cases, the number of pulses used in the centrifuge-N/MEP for producing extracellular vesicles is at least about 20 pulses. In some cases, the number of pulses used in the centrifuge-N/MEP for producing extracellular vesicles is at least about 30 pulses. In some cases, the number of pulses used in the centrifuge-N/MEP for producing extracellular vesicles is at least about 40 pulses. In some cases, the number of pulses used in the centrifuge-N/MEP for producing extracellular vesicles is at most about 10 pulses. In some cases, the number of pulses used in the centrifuge-N/MEP for producing extracellular vesicles is at most about 20 pulses. In some cases, the number of pulses used in the centrifuge-N/MEP for producing extracellular vesicles is at most about 30 pulses. In some cases, the number of pulses used in the centrifuge-N/MEP for producing extracellular vesicles is at most about 40 pulses.

The extracellular vesicles produced as described herein can be different types of extracellular vesicles. In some cases, the produced extracellular vesicles are exosome, microvesicle, apoptotic body, or any combination thereof.

Also provided herein are methods of treating a subject in need of comprising administering an effective amount of the extracellular vesicles produced as described herein. In some cases, the methods comprise administering to the subject via an intravenous, intramuscular, or subcutaneous route. In some cases, the methods comprise administering to the subject the extracellular vesicles produced as described herein in combination with other standard therapies.

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various cases may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a centrifuge tube” includes a plurality of centrifuge tubes, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value. Similarly, when the term “about” is used before a non-numerical term that is a stand-in for a numerical value (e.g., horizontal, perpendicular, aligned), the term “about” refers to the value of the non-numerical term (e.g., 90 degrees, 1800 degrees) plus or minus 10% of that value.

Whenever the term “at least,” “greater than,” “greater than or equal to,” “no more than,” “less than,” or “less than or equal to” precedes the first numerical value in a series of two or more numerical values, the term term applies to each of the numerical values in that series of numerical values, unless otherwise specified. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “bases,” as used herein refers to nucleotides. In some cases, “bases” can refer to base pairs (“bp”), e.g. 1 base equals 1 base pair. As used herein, the terms, “bases,” and “base pairs,” are used interchangeably.

As used herein, the term “about” means within 10% above or below a given value. For example, “about 10”, would include values from 9 to 11, unless otherwise indicated by the context in which the term is used.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The term “cycle” used herein may refer to one iteration from loading cells to be electroporated to the centrifuge-N/MEP setup to collecting the cells after centrifuge and electroporation. The term “cycle time” used herein refers to the duration of time in one cycle.

The section headings used herein are for organizational purposes only and are not to be construed as limiting the subject matter described.

### VII. EXAMPLES

The following examples are included for illustrative purposes only and are not intended to limit the scope of the invention.

### Example 1: Design of the Centrifuge-N/MEP and a Procedure Thereof

As illustrated in FIG. 1, the schematics of the centrifuge-N/MEP is shown. The N/MEP device was integrated in a commercial centrifuge tube. The N/MEP chip was sandwiched between the upside cell suspension and bottom side cargo solution. A slip ring connector, connecting the electrodes on the tubes and the external power supply, rotated together with the rotor in the centrifuge while keep the whole electro circuit connected. The N/MEP transfection was performed while the centrifuge continued in order to keep holding the cells against nanopores on the N/MEP chip surface via the centrifugal forces.

Transfection reagents loaded in the reservoir under the N/MEP chip were electrophoretically injected into nanoporated and individually positioned cells by applying a focused electric field through the nanochannels.

After N/MEP, the transfected cells were released, collected and incubated elsewhere. Fresh cells could be reloaded into the centrifugal tube and the centrifugal N/MEP cycle might start again. A cycle time could be less than 10 minutes.

A prototype centrifuge-N/MEP device was designed and built accordingly. The entire chassis of the centrifuge including the rotation frame was used as the cathode with the electric connector on the outer chassis surface. A platinum wire-based cathode was placed at the bottom of each electroporation tube and extended with copper foil on the outer tube surface. When the tube was placed into the centrifuge tube, the pre-placed copper foil stripes in and out of the centrifuge tube allowed the cathode to be in contact with the rotation frame.

On the rotation frame, a copper foil stripe on a double-sided tape is placed from each centrifuge tube location to the central screw location. A plastic holder with copper foil strips on the outer surface and an electrical socket on the top of the holder was tightly mounted onto the central screw. The copper foil stripes were welded to the socket. On the centrifuge cover, an electric plug was placed on the inner surface such that the plug was in contact with the socket when the cover is closed. Anode was formed on the outer surface of the cover.

The nanoporation centrifuge tube was designed in such a way that the anode was a long metal wire via the tube cap with a platinum electrode located at the lower end of the metal wire. Outside the centrifuge tube cap, a metal spring was attached to the metal wire such that the spring would be in contact with the copper foil stripe on the rotation frame when the tube is in a horizontal position under rotation. This wireless centrifuge design not only provides operation stability and user-friendliness, but is also suitable for scaling-up. Once the centrifuge started, the tube bucket swinged up and the spring electrode was in contact with the copper foil connecting to the external power input, which triggered the whole electric circuit. Electrically conductive silver paste was placed in the socket and the bottom of the centrifuge tube to improve electrical connection with low resistance.

The 12-mm-sized Transwell membrane holders were fixed near the lower portion of the centrifuge tube. As shown in FIG. 1E, the cell solution could be placed on top of the membrane holder, and the cargo solution was at the bottom reservoir of the centrifuge tube below the membrane holder.

The N/MEP-based transfection was performed while the centrifuge continued to keep holding the cells against nanopores on the N/MEP chip surface via the centrifugal forces. Transfection reagents loaded in the reservoir under the N/MEP chip were electrophoretically delivered into nanoporated individually positioned cells by applying a focused electric field through the nanochannels. Square wave electric voltage pulses (voltage 25 to 800 V, pulse duration 10 to 50 ms, 1 to 100 pulses depending on cell type and voltage) for nano-electroporation were generated from a power supply (e.g., Gene Pulser Xcell™, Bio-Rad).

After N/MEP, the transfected cells were quickly released and collected by a long-needle syringe, and incubated elsewhere. EVs including exosomes released from the transfected cells were collected from cell culture medium after a pre-specified incubation time. Fresh cells could be reloaded into the nanoporation centrifuge tube using the long-needle syringe and the centrifuge-N/MEP cycle might start again. The cycle time can be less than 10 minutes. Two nearly identical functional centrifuge-N/MEP devices and 8 workable electroporation centrifuge tubes have been successfully prepared.

### Example 2: Application of Centrifugal Force Improved Electroporation Efficiency

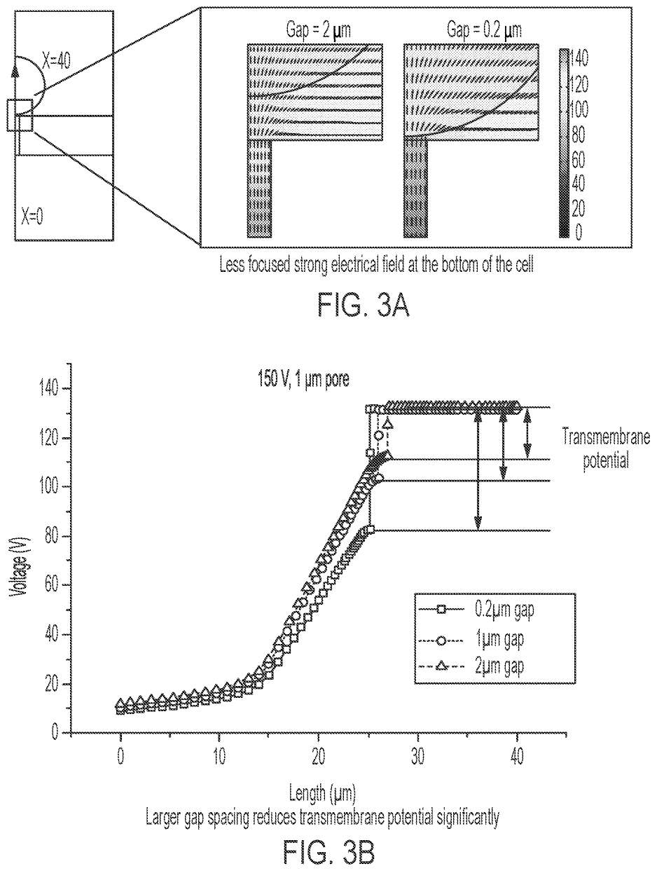

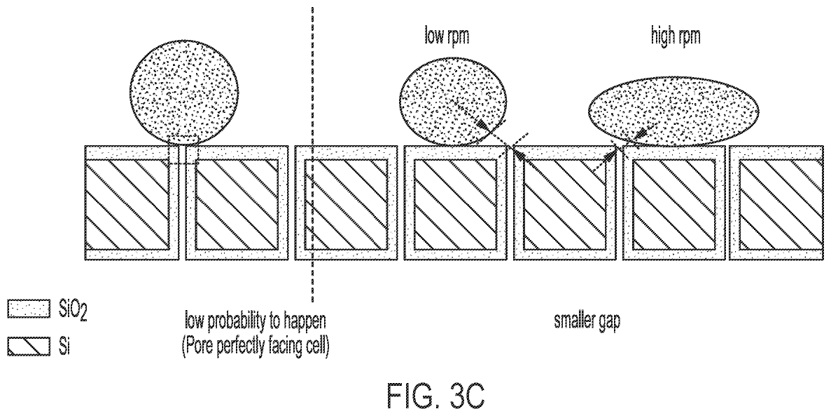

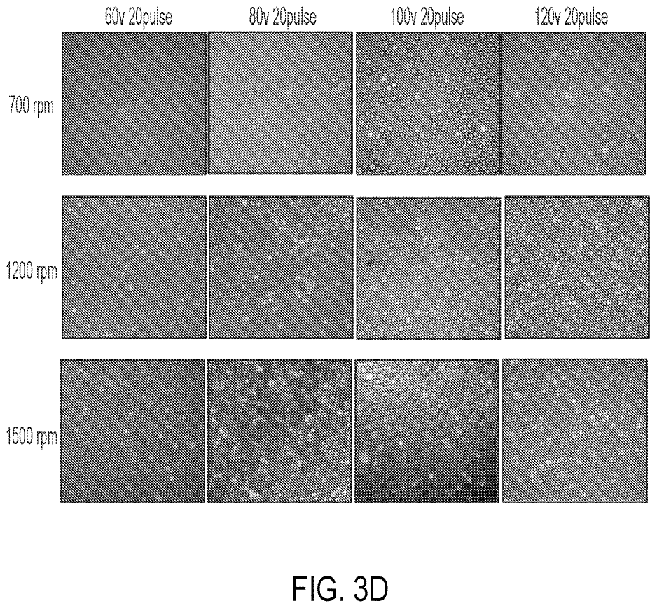

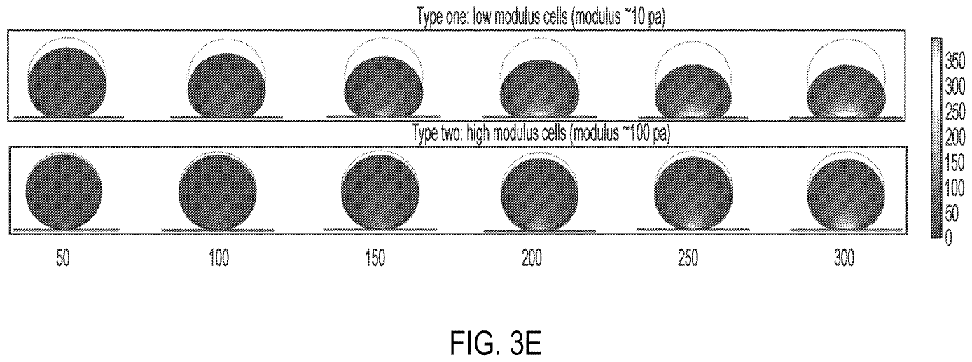

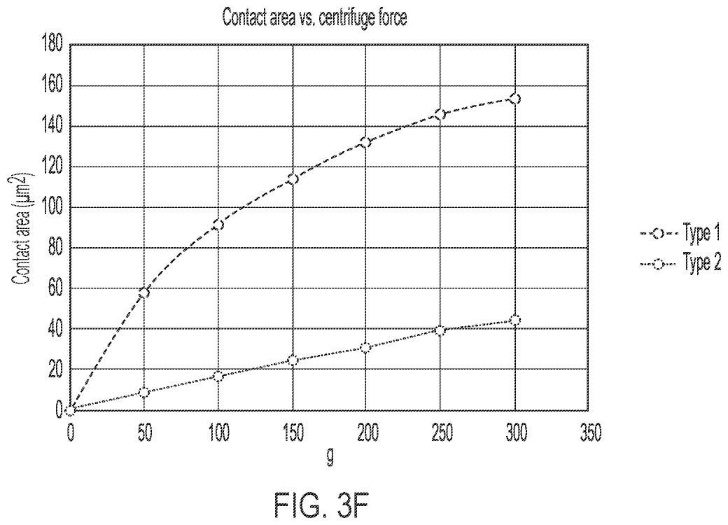

Proper positioning of a cell on or proximal to a nanopore of an electroporation chip was proven to greatly improve efficiency of electroporation. As depicted in FIG. 3A and FIG. 3B, when the gap between a cell and a nanopore of an electroporation chip became smaller, the transmembrane potential the cell experienced under the same external voltage applied was bigger, thereby increasing the electroporation efficiency.

Along with the phenomenon observed above, without centrifugation, there is a low probability where a cell is positioned perfectly on or proximal to the nanopore, thus the gap between the cell and the nanopore is usually large. However, under centrifugation, the gap between the cell and the nanopore gets smaller, when the cells are flattened or elongated against the surface of the electroporation chip under the centrifugal force. Therefore, with the rotations per minute (RPM) or relative centrifugal force (RCF) is increased, the gap between the cell and the nanopore is further shortened.

A transfection of small oligodeoxynucleotides (FAM-ODN) yielded consistent results (see FIG. 3D). A concentration of 250 ng/m of FAM-ODNs was prepared underneath the chip. Then centrifugal forces were applied. A cell population of a density of 500,000 cells per well was centrifugated for 5 minutes to ensure all of the cells were closer to the electroporation chip. In this experiment, varying voltages (60, 80, 100, or 120 volts) were applied in 10 millisecond (ms) pulses with a pulse intervals of 0.1 second for 50 minutes. The cells were centrifuged at varying speeds (700, 1200, or 1500 rpm) during the electroporation. At low rpm, the transfection efficiency was sub-optimal. In contrast, the transfection efficiency under both 1200 and 1500 rpm was increased. While significant improvement was shown between from 700 to 1200 rpm, limited further improvement was exhibited from 1200 rpm to 1500 rpm in the current experimental settings.

Different cell types may be flattened or elongated to different extents under centrifugal forces. As illustrated by FIG. 3D, the effects of centrifugal forces on the morphology of a cell with a low elastic modulus are different from the ones on the morphology of a cell with a higher elastic modulus in the bottom row. FIG. 3E quantitively shows the increase of contact area by an increase of higher centrifugal forces is more effective for type 1 cell. Therefore, N/MEP for cells with a similar property as type 1 may benefit more by the disclosed centrifuge-N/MEP design disclosed herein.

### Example 3: Small or Large Molecules Were Transfected via Centrifuge-N/MEP

Both small oligodeoxynucleotides (FAM-ODN) and large plasmid DNAs (GFP) were tested to be electroporated to Mouse Embryonic Fibroblasts (MEFs) successfully with centrifuge-N/MEP, respectively.

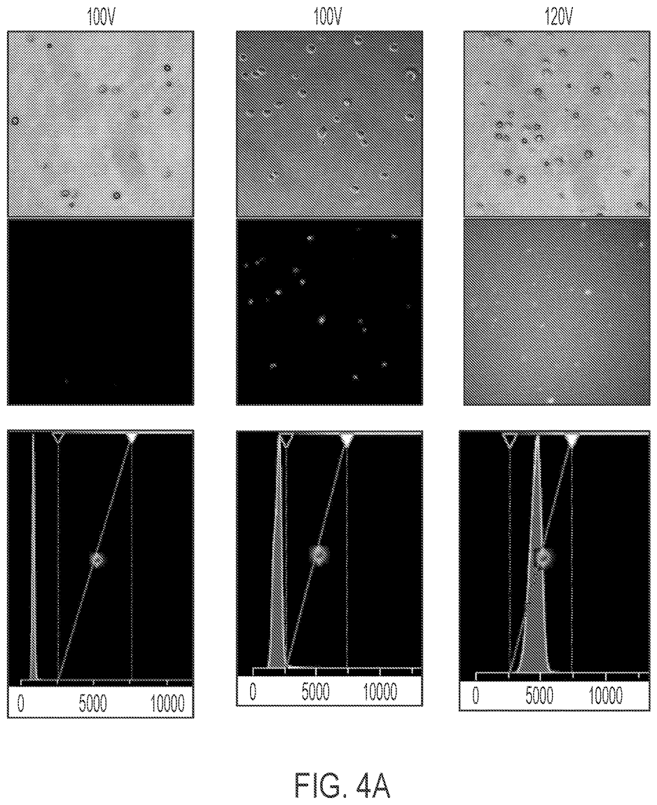

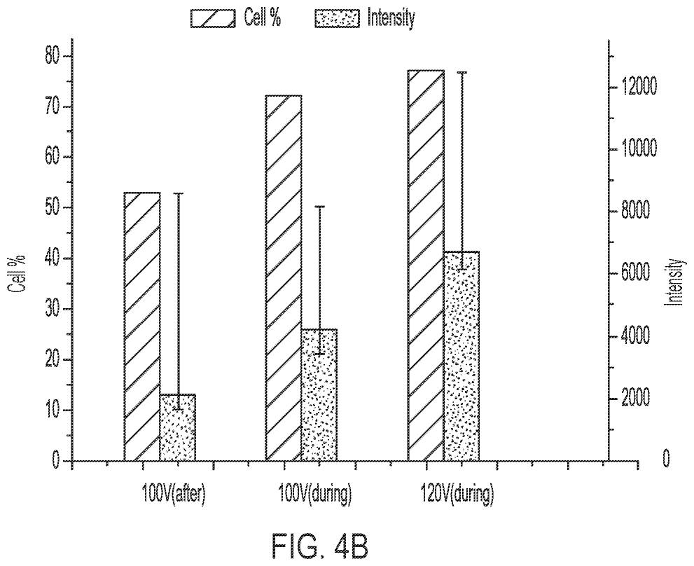

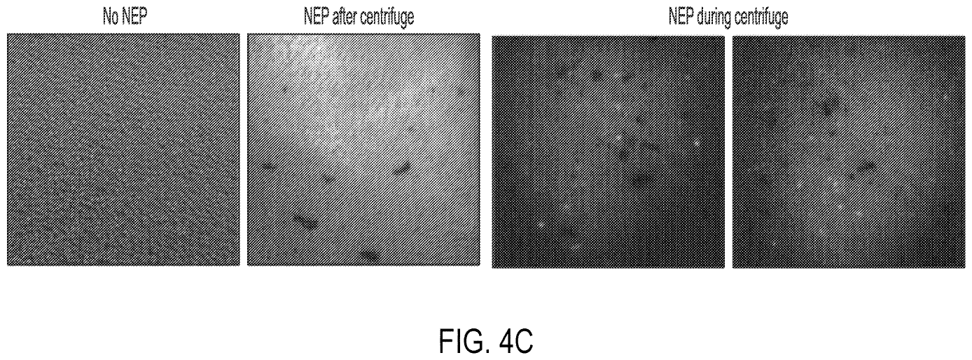

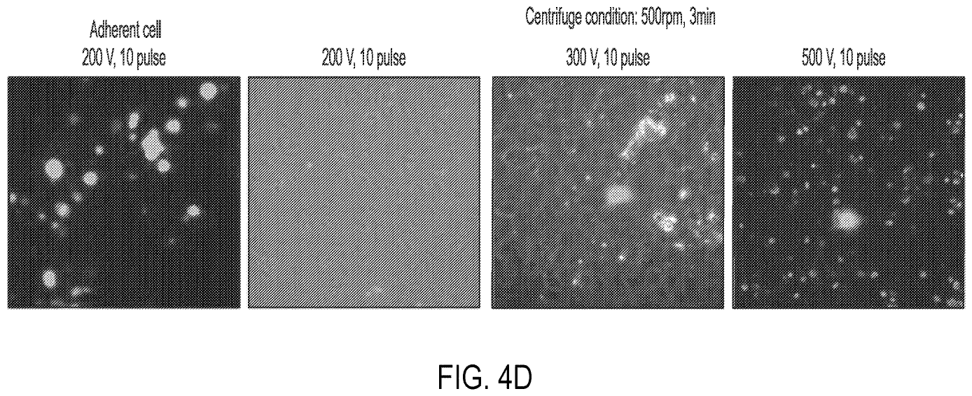

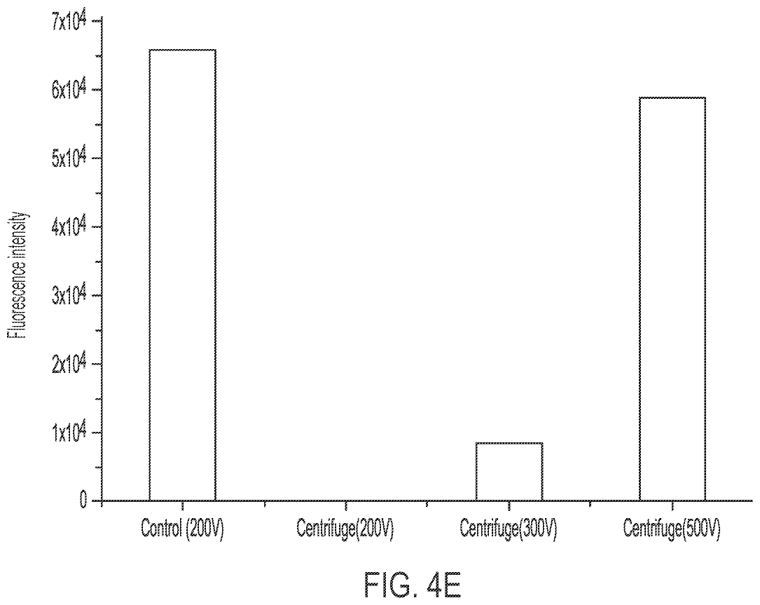

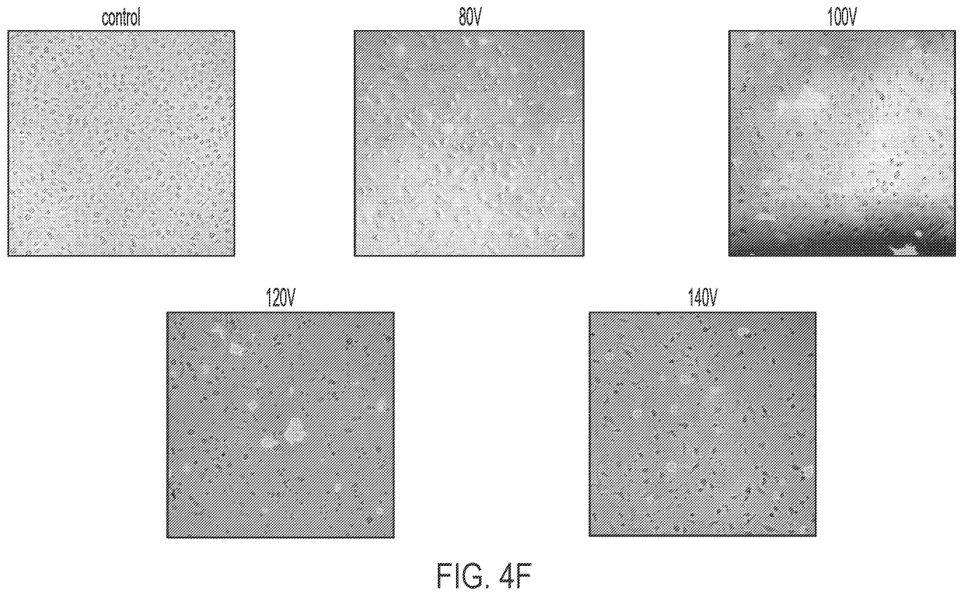

FIGS. 4A and 4B depict the electroporation of FAM-ODNs with a molecular weight about 500 g/mol during and after centrifuging. The percentage of the transfected cells and the fluorescence intensity of the transfected cells were improved when compared the N/MEP after centrifuge versus during centrifuge, which is consistent with the observation in Example 2: centrifugal forces lead to better contact between the nanochannel and the cell surface. The percentage of the transfected cells and the fluorescence intensity of the transfected cells were also improved when the voltage increased from 100V to 120V.

Similarly, as illustrated in FIG. 4C, the FAM-ODNs were transfected with slightly different configurations but with success. Specifically, the centrifuge conditions were set as 500 rpm for 3 min, and the electroporation condition was set as 300 V with 10 pulses (10 ms plus length, 0.1 s pulse interval).

Importantly, the centrifuge-N/MEP has been proven to be versatile in terms of the different sizes of transfection agents. For example, large-sized GFP plasmid with a molecular weight about 30,000 g/mol was also successfully delivered into MEFs by centrifuge-N/MEP (see FIG. 4D). The centrifuge conditions in the experiment were set as 500 rpm, 3 min, and the optimized electroporation conditions were set with 10 pulses (10 ms pulse length, 0.1 s pulse interval). Due to the additional resistance introduced by the centrifuge-N/MEP device (e.g., contact resistance), the optimized electroporation voltage was investigated among 200V, 300V, and 500 V. When the external voltage reached 500 V, the fluorescence intensity of the transfected cells (about 6×10⁴) became close to that of the adherent cells, showing similar dosage of cell transfection (see FIG. 4E). A track-etched membrane (12 mm sized Transwell) was used in this study as the electroporation chip. Similarly, GFP transfections were carried out under 80V, 100V, 120V, or 140V with centrifuge-N/MEP (see FIG. 4F).

### Example 4: Improvements in N/MEP Chips

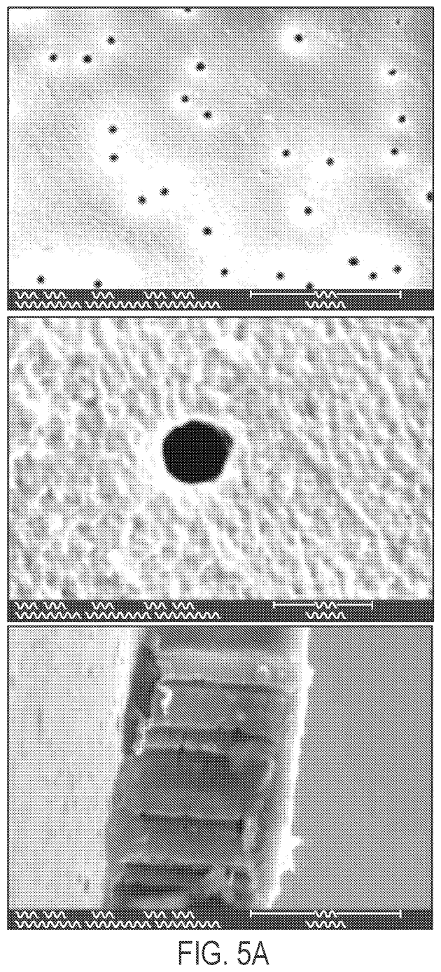

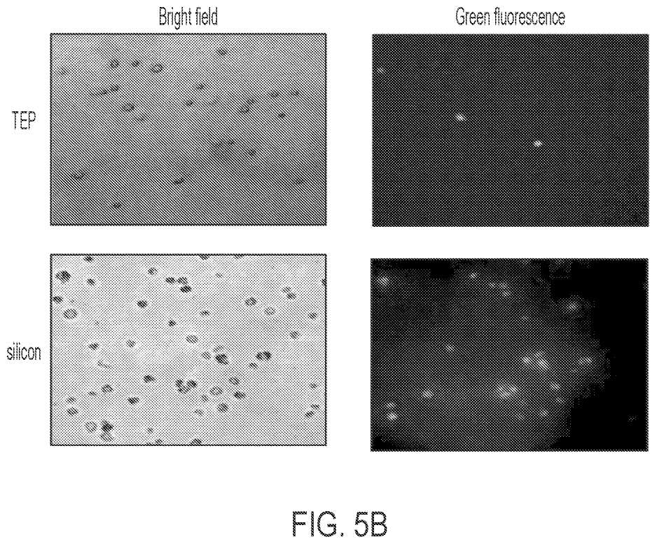

A track-etched membrane has a relatively uniform pore size (about 400 nm) and pore depth (about 10 μm), while the pore spacing was non-uniform with a lot of surface area without any pores (FIG. 5A). The percentage of cells transfected with GFP by centrifuge-N/MEP described above was only about 10%, which was much lower than that of the adherent cells (more than 70%). The low transfection percentage was also observed in the transfection of FAM-ODNs.

In contrast, the Si wafer-based N/MEP chips had a much better performance. FIG. 5B shows a comparison of FAM-ODN delivery to suspended MEFs on Transwell (TEP)- and Si wafer-based N/MEP chips using centrifugal forces to push MEFs against the chip surface. MEFs were nanoporated after centrifuge. Clearly, the Si wafer-based N/MEP chip could better transfect MEFs than the Transwell-based N/MEP chip.

### Example 5: Centrifuge-N/MEP Application in the Extracellular Vesicle Production

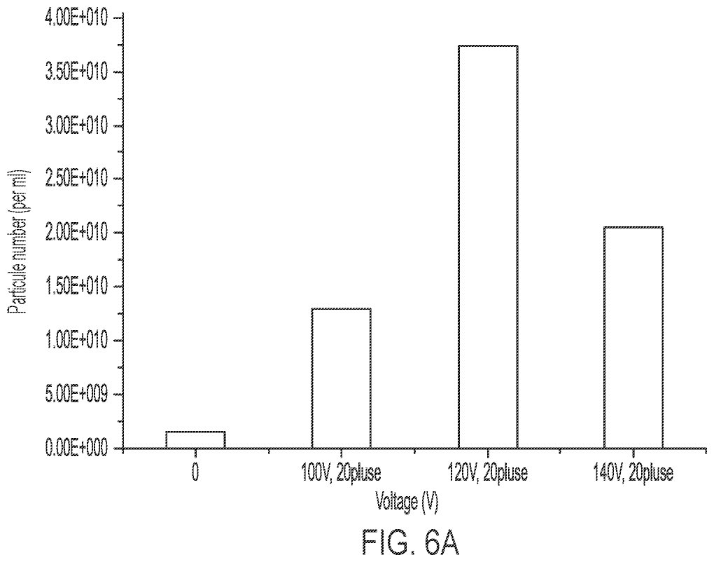

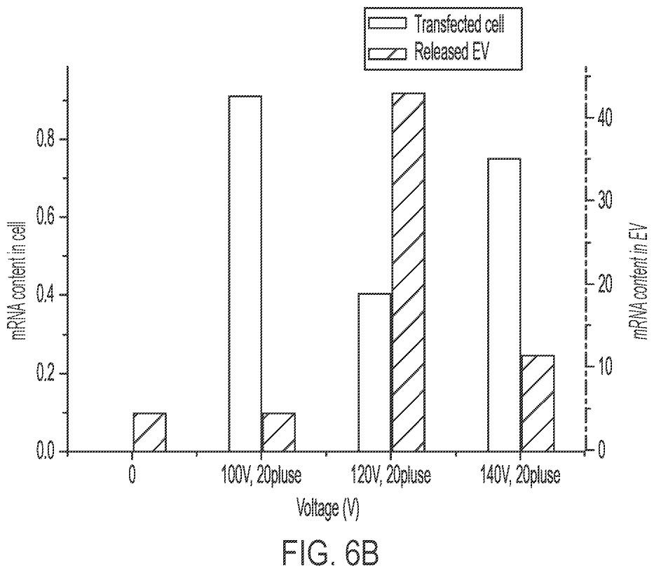

FIGS. 6A and 6B depict results of delivering P53 plasmid DNA into MEFs 24 hours after centrifuge-N/MEP conducted at varying voltages. The results show about a ten-fold increase in the EV number. Interestingly, an analysis of the location of mRNA content in the transfected cells versus in the released EVs shows that under 120V and 20 pulses under centrifuge-N/MEP the p53 mRNA was selectively enriched in released EVs.

While preferred cases of the present invention have been shown and described herein, it will be obvious to those skilled in the art that such cases are provided by way of example only. Numerous variations, changes, and substitutions will now occur to those skilled in the art without departing from the invention. It should be understood that various alternatives to the cases of the invention described herein may be employed in practicing the invention. It is intended that the following claims define the scope of the invention and that methods and structures within the scope of these claims and their equivalents be covered thereby.

1. A method of electroporating cells comprising:
a. providing a suspension comprising a plurality of cells to a first surface of an electroporation chip;
b. applying a centrifugal force to the plurality of cells, such that at least a portion of the plurality of cells is pressed against the first surface of the electroporation chip; and
c. providing an electrical voltage across the electroporation chip as the centrifugal force is applied to the plurality of cells.

2. The method of claim 1, wherein the electric voltage is provided when the first surface of the electroporation chip is about perpendicular to an axis of the centrifugal force.

3. The method of claim 1, wherein applying a centrifugal force to the plurality of cells flattens the portion of the plurality of cells against the electroporation chip.

4. The method of claim 1, wherein applying a centrifugal force to the plurality of cells elongates the portion of the plurality of cells against the electroporation chip.

5. The method of claim 1, wherein providing the suspension comprises injecting the suspension into a cell chamber adjacent to the first surface of the electroporation chip.

6. The method of claim 5, wherein the suspension is injected into the cell chamber using a syringe.

7. The method of claim 1, wherein the centrifugal force is applied using a centrifuge.

8. The method of claim 7, wherein the centrifuge comprises a rotor, and applying the centrifugal force further comprises rotating the rotor at approximately 100 to 2000 RCF.

9. The method of any one of claims 1 to 8, wherein the plurality of cells comprises a plurality of eukaryotic cells.

10. The method of claim 9, wherein the plurality of eukaryotic cells comprises mammalian cells.

11. The method of claim 10, wherein the mammalian cells are selected from the group consisting of human cells, rodent cells, and non-human primate cells.

12. The method of claim 9, wherein the plurality of eukaryotic cells comprises a plurality of cells from a cell line.

13. The method of claim 12, wherein the plurality of cells from a cell line is a plurality of cells from a suspension cell line or an adherent cell line.

14. The method of claim 13, wherein the suspension cell line is selected from the group consisting of a cell line of a myeloma origin, a cell line of a lymphoma origin, and a cell line of a leukemia origin.

15. The method of claim 14, wherein the suspension cell line is a human cell line.

16. The method of claim 14, wherein the suspension cell line is a non-human cell line.

17. The method of claim 9, wherein the plurality of eukaryotic cells is selected from the group consisting of mouse embryonic fibroblasts (MEF), human embryonic fibroblasts (HEF), dendritic cells, mesenchymal stem cells, bone marrow-derived dendritic cells, bone marrow derived stromal cells, adipose stromal cells, enucleated cells, neural stem cells, immature dendritic cells, and immune cells.

18. The method of claim 15, wherein the plurality of eukaryotic cells is a suspension cell line selected from the group consisting of NS0, U937, HL60, WEHI231, YAC1, U266B1, Jurkat, and THP-1.

19. The method of any one of claims 1 to 18, wherein the first surface of the electroporation chip comprises a plurality of pores.

20. The method of claim 19, wherein individual pores within the plurality of pores comprises a diameter within a range of about 50 nanometers to about 10 micrometers.

21. The method of any one of claims 1 to 20, wherein the current is applied at a voltage/distance between two electrodes of about 0.5V/cm to about 1000V/cm.

22. The method of claim 1, further comprising:
d. removing the suspension after providing the electrical voltage across the electroporation chip as the centrifugal force is applied to the plurality of cells.

23. The method of claim 22, further comprising repeating (a) to (d).

24. The method of claim 23, further comprising varying a voltage of the electrical current between repeating (a) to (d).

25. The method of claim 23 or 24, further comprising varying a duration of the electrical current between repeating (a) to (d).

26. A centrifuge for providing an electrical current to a suspension within one or more centrifuge tubes, the centrifuge comprising:
a rotor comprising a hub;
at least one tube holder connected to the hub; and
a circuit for providing an electric current and voltage through at least one of the one or more centrifuge tubes.

27. The centrifuge of claim 26, wherein at least one centrifuge tube, or each centrifuge tube, of the one or more centrifuge tubes comprises a first electrode and a second electrode for electric communication with the circuit.

28. The centrifuge of claim 26 or 27, wherein the at least one tube holder is rotatable about the hub.

29. The centrifuge of claim 28, wherein the at least one tube holder pivots under influence of a centrifugal force applied by the rotor.

30. The centrifuge of claim 29, wherein the electric current and voltage is provided through at least one centrifuge tube, or each centrifuge tube, of the one or more centrifuge tubes when a center axis of a centrifuge tube is aligned or about aligned with an axis of the centrifugal force applied to the one or more centrifuge tubes.

31. The centrifuge of claim 30, wherein at least one of the one or more centrifuge tubes comprises a first electrical contact, and wherein the circuit comprises a second electrical contact, wherein the first electrical contact and the second electrical contact are in electrical communication when the center axis of the centrifuge tube is aligned or about aligned with the axis of the centrifugal force applied on the one or more centrifuge tubes.

32. The centrifuge of any one of claims 26 to 31, wherein the circuit is connected to a power source.

33. The centrifuge of claim 32, wherein the power source is external to the centrifuge.

34. The centrifuge of claim 33, wherein the centrifuge further comprises a connector to provide electrical communication between the power source and the circuit.

35. The centrifuge of claim 32, wherein the power source is provided within the centrifuge.

36. The centrifuge of any one of claims 26 to 35, wherein the power source supplies a voltage of about 1V to about 500V to the circuit.

37. The centrifuge of any one of claims 26 to 36, wherein the rotor rotates at approximately 100 to 2000 RCF.

38. An electroporation system comprising:
one or more centrifuge tubes in which at least one of the one or more centrifuge tubes comprises:
a first tube,
an electroporation chip disposed within the first tube,
a first electrode provided on a first side of the electroporation chip, and
a second electrode provided on a second side of the electroporation chip;
a centrifuge device comprising:
a rotor comprising a hub;
one or more tube holders coupled to the hub; and
a circuit for providing an electric current to the one or more centrifuge tubes provided within the one or more tube holders; and
a power source for supplying the electric current to the circuit.

39. The electroporation system of claim 38, wherein at least one of the one or more tube holders are pivotably coupled to the hub such that the one or more tube holders rotate towards a horizontal orientation or towards about a horizontal orientation as the rotor is rotated.

40. The electroporation system of claim 39, wherein one or more first contacts of the centrifuge contact the first electrode of at least one of the one or more centrifuge tubes when the rotor is rotated.

41. The electroporation system of any one of claims 38 to 40, wherein the rotor is rotated at approximately 100 to 2000 RCF.

42. The electroporation system of claim 38, further comprising a cell chamber incorporated on the first side of the electroporation chip.

43. The electroporation system of claim 42, wherein the first electrode is provided within the cell chamber.

44. The electroporation system of claim 43, further comprising a first electrode wire for operatively connecting the first electrode to a first electrode contact, wherein the first electrode contact is provided on an exterior of the first tube.

45. The electroporation system of claim 44, wherein at least one centrifuge tube, or each centrifuge tube, of the one or more centrifuge tubes comprises a removable cap.

46. The electroporation system of claim 45, wherein at least one centrifuge tube of the one or more centrifuge tubes further comprises an aperture is provided through the removable cap, such that the first electrode wire passes through the aperture of the removable cap.

47. The electroporation system of claim 46, wherein at least one centrifuge tube, or each centrifuge tube, of the one or more centrifuge tubes further comprises a stabilizer, wherein the stabilizer comprises an electrode wire aperture such that the first electrode wire passes through the electrode wire aperture to provide an electrical connection from the first electrode to the first electrode contact.

48. The electroporation system of claim 47, wherein the stabilizer holds the first electrode wire in place.

49. The electroporation system of claim 42, wherein at least one centrifuge tube, or each centrifuge tube, of the one or more centrifuge tubes further comprises a second tube, wherein the second tube comprises a stabilizer which abuts the cell chamber and provides a physical barrier between the second tube and the cell chamber.

50. The electroporation system of claim 49, wherein the first electrode is provided within the cell chamber.

51. The electroporation system of any one of claims 38 to 50, wherein the centrifuge comprises a plurality of tube holders for simultaneous electroporation of one or more samples provided in a plurality of centrifuge tubes.

52. A method of using the electroporation system of claim 38, wherein the method comprises providing a suspension comprising a plurality of cells to the first side of the electroporation chip; applying a centrifugal force to the plurality of cells, such that at least a portion of the plurality of cells is pressed or elongated against the first surface of the electroporation chip; and providing an electrical voltage across the electroporation chip as the centrifugal force is applied to the plurality of cells.

53. A centrifuge tube comprising:
a first tube;
an electroporation chip disposed within the first tube;
a first electrode provided on a first side of the electroporation chip; and
a second electrode provided on a second side of the electroporation chip.
wherein the first electrode and the second electrode provide an electric field across the electroporation chip.

54. The centrifuge tube of claim 53, further comprising a cell chamber incorporated on a first side of the electroporation chip.

55. The centrifuge tube of claim 54, wherein the first electrode is provided within the cell chamber.

56. The centrifuge tube of claim 55, further comprising a first electrode wire for operatively connecting the first electrode to a first electrode contact, wherein the first electrode contact is provided on an exterior of the first tube.

57. The centrifuge tube of claim 56, further comprising a removable cap.

58. The centrifuge tube of claim 57, wherein an aperture is provided through the removable cap, such that the first electrode wire passes through the aperture of the removable cap.

59. The centrifuge tube of claim 58, further comprising a stabilizer, wherein the stabilizer comprises an electrode wire aperture such that the first electrode wire passes through the electrode wire aperture to provide an electrical connection from the first electrode to the first electrode contact.

60. The centrifuge tube of claim 59, wherein the stabilizer holds the first electrode wire in place.

61. The centrifuge tube of claim 60, further comprising a second tube, wherein the second tube comprises a stabilizer which abuts the cell chamber and provides a physical barrier between the second tube and the cell chamber.

62. The centrifuge tube of claim 61, wherein the first electrode is provided within the cell chamber.

63. The centrifuge tube of claim 61 or 62, further comprising a first electrode wire for operatively connecting the first electrode to a first electrode contact, wherein the first electrode contact is provided on an exterior of the first tube.

64. The centrifuge tube of claim 63, further comprising a removable cap.

65. The centrifuge tube of claim 64, wherein an aperture is provided through the removable cap, such that the first electrode wire passes through the aperture of the removable cap.

66. The centrifuge tube of claim 65, further comprising a second electrode contact electrically coupled to the second electrode and provided on an exterior to the first tube.

67. The centrifuge tube of claim 66, wherein the second electrode contact is provided on a side of the first tube opposite side of the first electrode contact.

68. The centrifuge tube of claim 66 or 67, wherein the first electrode is a positive electrode and wherein the second electrode is a negative electrode.

69. The centrifuge tube of claim 66 or 67, wherein the first electrode is a cathode and wherein the second electrode is an anode.

70. The centrifuge tube of claim 64 or 65, wherein the removable cap comprises a syringe aperture for receiving a syringe to provide a plurality of cells to the cell chamber.

71. The centrifuge tube of claim 70, wherein the syringe aperture is positioned off a center axis of the removable cap.

72. The centrifuge tube of claim 70 or 71 wherein the stabilizer comprises a syringe through hole for receiving the syringe to provide cells to the cell chamber.

73. The centrifuge tube of any one of claims 61 to 72, wherein a friction fit is provided between the stabilizer and the first tube.

74. The centrifuge tube of claim 73, wherein the friction fit facilitates retention of the second tube within the first tube.

75. The centrifuge tube any one of claims 61 to 74, wherein the stabilizer provides a liquid tight seal of the cell chamber.

76. The centrifuge tube of claim 68, wherein the stabilizer provides an air-tight seal of the cell chamber.

77. The centrifuge tube of claim 53, further comprising a transfection reagent chamber located between the second side of the electroporation chip and the closed end of the first tube.

78. The centrifuge tube of claim 77, wherein the second electrode is within the transfection reagent chamber.

79. The centrifuge tube of claim 64, wherein the open end of the first tube is threaded to receive the removable cap.

80. The centrifuge tube of claim 64, further comprising a first electrode wire for operatively connecting the first electrode to a first electrode contact, wherein the first electrode contact is provided on an exterior of the removeable cap, and wherein an aperture is provided through the removable cap, such that the first electrode wire passes through the aperture of the removable cap.

81. The centrifuge tube of claim 65, further comprising a second electrode contact electrically coupled to the second electrode and provided at the exterior of the first tube and proximal to the closed end of the first tube.

82. A method of electroporating cells comprising:
a. providing a suspension comprising a plurality of cells adjacent to an electroporation chip;
b. elongating the plurality of cells, such that at least a portion of the plurality of cells is pressed against a first surface of the electroporation chip; and
c. providing an electrical current across the electroporation chip.

83. A method of electroporating cells comprising:
a. providing a suspension comprising a plurality of cells adjacent to a surface of or within a chamber of an electroporation chip;
b. flattening the plurality of cells, such that at least a portion of the plurality of cells is pressed against a first surface of the electroporation chip; and
c. providing an electrical current across the electroporation chip.

84. The centrifuge tube of claim 53, wherein a distal portion of the first tube comprises a transfection reagent chamber to receive a transfection reagent.

85. The centrifuge tube of claim 84, wherein the transfection reagent comprises a plasmid, DNA, RNA, protein, other charged biomolecules, charged molecules, charged particulates, or a combination thereof.

86. The centrifuge tube of claim 53, wherein the electroporation chip provides a physical barrier between the transfection chamber and the cell chamber.

87. The centrifuge tube of claim 53, wherein the electroporation chip is removable.

88. The centrifuge of claim 85, wherein the first tube comprises an open end and a closed end.

89. The method of claim 1, wherein the providing the electrical voltage occurs after centrifugal movement resulting from the centrifugal force stops.

90. The method of claim 1, wherein the providing the electrical voltage occurs after centrifugal movement resulting from the centrifugal force begins to stop.

91. The method of claim 1, wherein the providing the electrical voltage occurs after the centrifugal movement resulting from the centrifugal force slows down to a velocity less than 50%, less than 25 %, or less than 10% of a highest velocity the centrifugal movement once reaches.

92. The method of claim 1, wherein the providing the electrical voltage occurs after the plurality of cells are elongated.

93. The method of claim 1, wherein the providing the electrical voltage occurs after the plurality of cells are elongated in the absence of the centrifugal force.

94. The method of claim 1, wherein the providing the electrical voltage occurs after the plurality of cells are flattened.

95. The method of claim 1, wherein the providing the electrical voltage occurs after the plurality of cells are flattened in the absence of the centrifugal force.

96. The method of claim 1, wherein the providing the electrical voltage occurs simultaneously with the centrifugal force being applied.
